(19) United States
(12) Patent Application Publication
(10) Pub. No.: US 2026/0321363 A1
(43) Pub. Date: Oct. 15, 2026

(54) PREDICTION OF PHARMACOKINETIC PROPERTIES OF CHEMICAL COMPOUNDS

(71) Applicant: Hoffmann-La Roche Inc., Little Falls, NJ (US)

(72) Inventors: Katrin GROEBKE ZBINDEN, Liestal (CH); Leonid Maximowitsch KOMISSAROV, Basel (CH); Nenad MANEVSKI, Riehen (CH); Lisa Bertha SACH-PELTASON, Lörrach, Baden-Wurttemberg (DE); Torsten Ruediger SCHINDLER, Basel (CH); Raya Simeonova STOYANOVA, Zürich (CH)

(21) Appl. No.: 19/108,410

(22) PCT Filed: Sep. 5, 2023
(86) PCT No.: PCT/EP2023/074320

§ 371 Date: Mar. 3, 2025

(30) Foreign Application Priority Data
Sep. 5, 2022 (EP) 22193964.8

(51) Int. Cl. G16C 20/70; G06N 3/045; G16C 20/30; G16C 20/50

(52) U.S. Cl. CPC G16C 20/70; G06N 3/045; G16C 20/30; G16C 20/50

(57) ABSTRACT

Prediction of pharmacokinetic properties of chemical compounds A computer-implemented method of generating a machine-learning model configured to predict the value of one or more pharmacokinetic parameters of a pharmaceutical compound having a chemical structure comprises: receiving training data comprising a plurality of records, each record comprising: data encoding the chemical structure of a pharmaceutical compound; and the values of one or more pharmacokinetic parameters obtained from measurements taken from a human or animal subject after administration of the compound to the human or animal subject; and training the machine-learning model using the training data. Computer-implemented methods of utilizing the generated machine-learning model, particularly in a drug design context, are also provided.

### TECHNICAL FIELD OF THE INVENTION

The present invention relates to the use of machine-learning to predict pharmacokinetic properties of chemical compounds based on their structure. Corresponding computer-implemented methods, systems and the like are also provided.

### BACKGROUND TO THE INVENTION

PK properties such as plasma clearance (CLp), volume of distribution at steady state (Vss), and oral bioavailability (F) play a major role in drug efficacy and safety. Medicinal chemistry and multidisciplinary drug design teams have a longstanding interest in computational predictions of PK to minimize the number of design cycles, speed up the discovery process, reduce reliance on animal models (Madden et al., 2020), and to discover candidate molecules that can succeed in clinical studies (Cumming et al., 2013; Vamathevan et al., 2019).

To save valuable resources and animal lives, predictions are most desired at the drug design stage, even before compounds are synthesized. Despite the benefits, predictions of PK remain highly challenging, especially when experimental data is absent and only the chemical structure can be used to forecast future in vivo outcomes. Simple molecular properties, like lipophilicity and polar surface area, were historically used for probabilistic assessments and druggability guidelines, but this often led to broad trends with numerous exceptions (Lipinski, 2004a; Lombardo et al., 2018a; Poongavanam et al., 2018).

Since the year 2000, helped by accumulation of large in vitro datasets across the industry, as well as emergence of machine learning (ML) methods, computational predictions of individual absorption, distribution, metabolism, and excretion (ADME) properties, such as solubility, permeability, plasma protein binding, and metabolic stability, became widely used in drug discovery. Although this represents a major step forward, functional (in vivo) PK outcomes often remain elusive, driven by complex interplay of individual ADME properties with physiological systems.

Therefore, to enhance the speed and quality of drug design, there is a strong need to further develop computational methods to predict PK properties (Danishuddin et al., 2021).

This is an emerging scientific field where smaller dataset sizes, experimental uncertainty, and study design variability (e.g. dose, formulation, sampling times) offer a considerable challenge. In addition to prediction accuracy, the overall context of human interpretability and trust is equally important, including applicability domains, quantification of uncertainty, and explainability of predictions.

Currently, in vivo PK properties can be computationally predicted (1) directly, by developing ML methods based on observed PK results, or (2) indirectly, by first predicting individual in vitro ADME properties and then performing an in vitro-in vivo extrapolation, most often via physiologically-based pharmacokinetic models (PBPK) (see Table 1 for an overview of existing approaches). Whereas indirect approaches benefit from larger in vitro datasets and transparency of input parameters (Naga et al., 2022), the underlying assumption—that all PK outcomes are governed by a small subset of selected in vitro ADME properties—is rarely valid at the drug design stage, especially for diverse and experimental chemotypes that undergo extrahepatic metabolism and transporter-mediated disposition.

Further, for a set of predicted ADME inputs, PBPK models output a unique solution of differential equations, without error propagation, uncertainty quantification, and chemical explainability. On the other hand, direct approaches leverage historical PK data to establish an immediate link between PK observations and molecular features (Wang et al., 2019a; Miljković et al., 2021; Obrezanova et al., 2022). Although existing PK datasets are often limited in size and chemical diversity (≲3,000 compounds), especially the clinical ones (≲2,000), direct approach provides an opportunity to learn from decades of historical data and compare-and-contrast various ML methods.

A number of (3) hybrid approaches may address existing limitations of direct and indirect methods, especially by leveraging combined strengths of in vitro and in vivo datasets. For example, hybrid approaches include pretraining of direct ML models with in vitro data (Ye et al., 2019) and indirect PK predictions (Schneckener et al., 2019a), or by developing multitask ML models that can predict both in vitro and in vivo outcomes (Ye et al., 2019). A survey of the existing literature showed that while all computational approaches show promise (Table 1), further development is needed before these models could be applied in practice by drug design teams.

Notably, most existing studies rely on relatively small publicly available datasets (<3000 compounds), predict only a subset of PK properties (e.g. predominantly Vss, with fewer predictions of CLp, &F, and AUC), and lack critical user-feedback in terms of applicability domains and prediction errors (Table 1). Further, the central paradigm of rational drug design—the causal link between molecular features and physiological outcomes—is often missing, rendering these models largely inexplicable and intractable (Jiménez-Luna et al., 2020).

### SUMMARY OF THE INVENTION

To address many of the existing challenges, the present inventors explored methodology for direct and hybrid computational predictions of mouse and rat PK properties, focusing on both prediction accuracy and human interpretability of outcomes (applicability domain, quantification of uncertainty, and explainable AI) (FIG. 1). To avoid methodological bias towards isolated properties, our prediction targets were several common PK properties after IV and PO drug administration, including CLp (mL/min/kg), Vss (L/kg), oral F (%), and dose-normalized areas under the plasma concentration-time curves (AUCiv and AUCpo; h*ng/ml/mg/kg). We worked on a historical dataset of mouse and rat PK studies available at Roche (≅10,000 unique compounds tested in ≅30,000 PK studies). After developing an automated data preprocessing pipeline, we compared-and-contrasted various ML approaches, including single- and multi-task modes and pretraining with in vitro data. Prediction accuracy was assessed across different data splits, including random, scaffold (Schuffenhauer et al., 2007), SCINS (Bender et al., 2009), and time, and the applicability domain was visualized using the UMAP dimensionality reduction technique (Sainburg et al., 2020). The human interpretability of ML predictions was tested with the SHAP post-hoc approach (Lundberg and Lee, 2017), highlighting molecular features that contribute to predicted outcomes.

At a high-level, the present invention is directed towards a computer-implemented method of generating, and using a machine-learning model configured to predict the value of one or more pharmacokinetic parameters of a pharmaceutical compound having a chemical structure.

Specifically, a first aspect of the invention provides a computer-implemented method of generating a machine-learning model configured to predict the value of one or more pharmacokinetic parameters of a pharmaceutical compound having a chemical structure, the computer-implemented method comprising: receiving training data comprising a plurality of records, each record comprising: data encoding the chemical structure of a pharmaceutical compound; and the values of one or more pharmacokinetic parameters obtained from measurements taken from a human or animal subject after administration of the compound to the human or animal subject; and training the machine-learning model using the training data. In this manner, it is possible to derive pharmacokinetic parameters from data about the chemical structure alone, rather than requiring recourse to in vitro and in vivo experimentation at an early stage of the drug design process. This, in turn, enables streamlining of the drug design and development process. In some cases, the computer-implemented method may explicitly be implemented as part of the drug design process. More detailed about this is provided with respect to the third aspect of the invention.

As outlined earlier, the one or more pharmaceutical parameters may comprise one or more of:
Plasma clearance (CLp)—this has units of volume/time, and is a measure of the overall ability of the body to eliminate a drug, by scaling the drug elimination rate (amount per time) by a corresponding plasma level. This is an important pharmacokinetic parameter, since it quantifies the rate of drug elimination from the body. Together with volume of distribution at steady-state (Vss) and oral bioavailability (F), it provides means to predict drug doses, dosing regimens, and corresponding plasma exposures needed for therapeutic efficacy (for a given bioavailability, on which more shortly), and it is the parameter which enables computation of a dosage required to maintain a steady-state plasma concentration.
Volume of distribution at steady state (Vss)—Vss represents a theoretical volume of drug distribution at steady-state. Physiologically it represents a balance of drug's nonspecific binding between various tissues and plasma proteins. Together with CLp and F, it is a key PK parameter to dose and regimen needed for therapeutic efficacy. It is a useful parameter due to this physiological meaning.
Oral bioavailability (F)—this is the percentage or proportion of an orally administered drug which reaches systemic circulation. This in contrast to e.g. intravenous administration of a drug, since in those cases, all of the drug enters the circulatory system.
Area under the plasma concentration curve after intravenous dosing (AUCiv)—this is the definite integral of the concentration of a pharmaceutical compound in blood plasma over a certain time period after intravenous administration. Generally, the AUCiv provides a useful metric of the total drug exposure over time, and can be useful for determining whether e.g. two different formulations of the same dose result in equal exposures.
Area under the plasma concentration curve after oral dosing (AUCpo)—as above, except after oral (“per os”) administration.

In some cases, it may be beneficial to pre-process the training data to ensure that it is in a form which is most appropriate for training the machine-learning model. There are various pre-processing steps, any, some, or all of which may be employed in computer-implemented methods according to the first aspect of the present invention:
Preprocessing the training data may comprise removing records for which dosage levels were greater than a predetermined maximum threshold dosage level. The rationale behind this preprocessing step is that records where the dosage levels are very high are likely to have been part of toxicokinetic studies and safety evaluations, rather than pharmacokinetic measurements.
In implementations in which the pharmacokinetic parameter comprises AUCiv and/or AUCpo, preprocessing the training data may comprise extrapolating the AUCiv and/or AUCpo values from zero to infinity. In other words, the extrapolated AUCiv and/or AUCpo values represent the definite integral of the concentration-time curve from the initial dosing to infinity. In these cases pre-processing the data may further comprise, for those records for which the extrapolation factor was greater than a predetermined threshold (e.g. 1.5, 2, 3, 4, 5, 6, 7, 8, 9, or 10, but preferably 2), using the observed zero-to-last measurement (i.e. without extrapolating). Herein, the “extrapolation factor” is the value by which the value of the AUCiv and/or AUCpo is multiplied when extrapolating the curve to infinity.
Preprocessing the training data may comprise removing records for which measurements of the value of the one or more pharmacokinetic parameters were obtained from a bodily fluid other than blood or plasma.
Processing the training data may comprise removing records for which the molecular weight of the pharmaceutical compound is greater than a predetermined molecular weight threshold. For example, the predetermined maximum molecular weight threshold may be 1000 Da (or unified atomic mass unit).
In implementations in which the one or more pharmacokinetic parameters comprise F, preprocessing the training data may comprise removing records for which the value of the oral bioavailability parameter is greater than a predetermined maximum oral bioavailability threshold. This may comprise limiting the value of F to 0 to 100%. Alternatively, this may comprise winsorizing the values of F to 0 to 100%.
Preprocessing the training data may comprise normalizing the distribution of values of one or more of the pharmacokinetic parameters. Normalizing the distribution of values of one or more of pharmacokinetic parameter may comprise applying a statistical transformation to the values in the distribution of values. For example, normalizing the distribution of values of one or more of CLp, Vss, AUCiv, or AUCpo may comprise applying a log₁₀ transformation to the values of the pharmacokinetic parameter in each of the plurality of records of the training data. Alternatively or additionally, normalizing the distribution of values of F may comprise applying a logit function to the values of F in the plurality of records in the training data¹.
Preprocessing the training data may comprise removing outliers from the distribution of values of one or more of the pharmacokinetic parameters. More specifically, where replicate pharmacokinetic measurements are available in the training data, preprocessing data may comprise removing outliers whose value is no less than two standard deviations away from the mean.
Naturally, the training data will comprise a large number of different chemical compounds. The representations of these compounds may vary. For example, the data for some may be provided in salt form. In order to address this, preprocessing the training data may comprise standardizing the chemical structures of the pharmaceutical compounds in the training data. standardizing the chemical structures may comprise one or more of: removing salts; sanitization; removal of hydrogen atoms; disconnecting metals; normalization; reionization; and assigning stereochemistry. In the present context, sanitization refers to a plurality of operations which may be executed using the RDKit². The operations may include one, a subset, or all of the following:
   a) Removing any computed properties that already exist on the molecule and its atoms and bonds.
   b) Sstandardizing a small number of non-standard valence states. The clean-up operations may include:
      Neutral 5 valent Ns with double bonds to Os are converted to the zwitterionic form. Example: N(═O)═O->[N+](═O)O—]

$${}^{1} logit ( x ) = ln ( \frac{x}{1 - x} )$$

$${}^{2} https : // www . rdkit . org / docs / RDKit Book . html$$

Neutral 5 valent Ns with triple bonds to another N are converted to the zwitterionic form. Example: C—N═N #N->C—N═[N+]═[N−]
Neutral 5 valent phosphorus with one double bond to an O and another to either a C or a P are converted to the zwitterionic form. Example: C═P(═O)O->C═[P+]([O−])O
Neutral Cl, Br, or I with exclusively 0 neighbors, and a valence of 3, 5, or 7, are converted to the zwitterionic form. This covers things like chlorous acid, chloric acid, and perchloric acid. Example: O═Cl(═O)O->[O−][Cl+2][O−]O
c) Calculating the explicit and implicit valences on all atoms. This generates exceptions for atoms in higher-than-allowed valence states.
d) Calling the symmetrized smallest set of smallest rings algorithm.
e) Converting aromatic rings to their Kekule form. Will raise an exception if a ring cannot be kekulized or if aromatic bonds are found outside of rings.
f) Determining the number of radical electrons (if any) on each atom.
g) Identifying the aromatic rings and ring systems, setting the aromatic flag on atoms and bonds, setting bond orders to aromatic.
h) Identifying which bonds are conjugated.
i) Calculating the hybridization state of each atom
j) Removing chiral tags from atoms that are not sp3 hybridized.
k) Adding explicit Hs where necessary to preserve the chemistry. This is typically needed for heteroatoms in aromatic rings. The classic example is the nitrogen atom in pyrrole.
Pharmaceutical compounds are usually large, i.e. they have a molecular weight >100. To that end, preprocessing the training data may comprise removing pharmaceutical compounds whose chemical structures include fewer than three atoms. Alternatively or additionally, the step may also comprise removing pharmaceutical compounds having a molecular weight less than a predetermined minimum molecular weight threshold. As set out above, the minimum molecular weight threshold may be 100 Da.
Preprocessing the training data may comprise removing pharmaceutical compounds whose chemical structures display significant secondary structure. For example, this may be achieved by removing hexapeptides, e.g. by searching for a NCC(═O)NCC(═O)NCC(═O)NCC(═O)NCC(═O)NCC═O substructure.

We now discuss the kinds of machine-learning model which may be generated according to the computer-implemented method of the first aspect of the invention. The machine-learning model may comprise one or more of a gradient boosting model, an automatic machine-learning model, a Gaussian process regression (GPR) model, a support vector regression (SVR) model, or a random forest model. Examples of specific kinds of machine-learning models of these forms, and references to those models are provided in the “Experimental Results” section of this patent application.

In the above cases, the data encoding the chemical structure of each pharmaceutical compound may comprise one or more molecular fingerprints generated based on the chemical structure of that pharmaceutical compound. The computer-implemented method may comprise generating the molecular fingerprints, preferably after preprocessing of the training data has taken place. There are various kinds of molecular fingerprints which may be used to represent the chemical structures in question. In preferred cases, the molecular fingerprints comprise a string of characters which uniquely define the chemical structure of the pharmaceutical compound. These molecular fingerprints may be generated using standard approaches. For example, the molecular fingerprints may be in the form of an extended-connectivity fingerprint (such as an ECFP4 or ECFP6), a functional-connectivity fingerprint (such as an FCFP4 or FCFP6), a Morgan fingerprint, a Morgan2 fingerprint, or an MAP4 fingerprint.

In addition to a molecular fingerprint, the data encoding the chemical structure of each pharmaceutical compound may further comprise one or more molecular descriptors.

In other cases, rather than using one of the examples of a machine-learning model as set out above, the machine-learning model may be a graph neural network. Examples of graph neural networks which may be used include graph convolutional neural networks (GCNN); Attentive Fingerprints; and ChemProp. An Attentive Fingerprint model may comprise a GNN architecture that represents molecules using molecular fingerprints and a graph attention mechanism. A ChemProp model may comprise a message passing neural network for molecular property prediction. In implementations in which the machine-learning model is a graph neural network, the data encoding the chemical structure of each pharmaceutical compound preferably comprises a graph structure (i.e. rather than e.g. a molecular fingerprint). When graph neural networks are used, it is not necessary to provide the input in a standardized form. The graph structure preferably comprises nodes and edges, each node and edge having values associated therewith, the values defining the chemical properties of the atoms and bonds within the molecule.

Rather, the graph neural network is able to learn a representation of the chemical structure of the pharmaceutical compound in terms of hidden variables or hidden features. In the context of machine-learning processes, a hidden representation is a feature in terms of which a set of input data may be parameterized, but which may not be directly observable or obvious to the human eye. Accordingly, the graph neural network may comprise a first sub-network which is configured to generate a representation of the chemical structure in terms of hidden variables. Having generated the hidden representation, the graph neural network may further comprise a second sub-network configured to determine the values of the one or more pharmacokinetic parameters based on the generated representation in terms of hidden variables.

When using a graph neural network which is configured to learn its own representation of the chemical structure, as outlined above, improvements to the results can be obtained by pretraining the neural network before exposing it to the training data. Herein, “pretraining” refers to a process in which some or all of a neural network is trained using data which is different from the data which is used for the main training step. Accordingly, the computer-implemented method may further comprise, before the step of training the machine-learning model, pretraining the machine-learning model using pretraining data. More specifically, the computer-implemented method may comprise receiving pretraining data, the pretraining data comprising a plurality of records, each record comprising: data encoding the chemical structure of a pharmaceutical compound; and the values of one or more parameters obtained from in vitro measurements, or the values of one or more parameters obtained using machine-learning predictions of in vitro measurements. Pretraining the machine-learning model may then comprise pretraining the machine-learning model using the pretraining data before training the machine-learning model using the training data.

When pretraining the machine learning model comprises training the first sub-network and the second sub-network using the pretraining data, the computer-implemented method may subsequently comprise training both the first sub-network and the second sub-network using the training data.

When the machine-learning model is a graph neural network having the first sub-network and second sub-network as explained previously, pretraining the machine-learning model may comprise training the first sub-network and the second sub-network using the pretraining data. Then, an initial learning rate of the first sub-network may be reduced to a reduced learning rate of the first sub-network Herein, the term “learning rate” takes its usual meaning in the art, namely referring to a value, which is usually between 0 and 1, and which is a tuning parameter in an optimization algorithm defining a step size at each iteration while moving toward a minimum of a loss function. For example, the learning rate may refer to an amount (i.e. a proportion) that the weights of a model are updated during each iteration during training.

The initial learning rate may be in the range 0.1 to 0.000001, in the range 0.01 to 0.00001, or more preferably in the range 0.001 to 0.0001. The reduced learning rate may be reduced by a factor of 2 to 10,000, by a factor of 5 to 1,000 or more preferably by a factor of 10 to 100. For example, the reduced learning rate may be in the range from 10⁻⁶ to 10⁻⁵.

After the pre-training, the learning rate of the first sub-network may be lower than the learning rate of the second sub-network. The reduced learning rate of the first sub-network may have the values outlined above, and the learning rate of the second sub-network may be in the range 0.1 to 0.000001, in the range 0.01 to 0.00001, or more preferably in the range 0.001 to 0.0001.

The learning rate of the first sub-network and/or the learning rate of the second sub-network after the pre-training may be determined by trial and error. After the pre-training, the computer-implemented method may comprise training the neural network using the training data.

The learning rate of the first sub-network may be reduced to zero, i.e. the first-sub network may be frozen. In this case, training the graph neural network using the training data comprises training the second sub-network only using the training data. That is, after the pre-training step the first sub-network may be frozen, and the computer-implemented method may comprise training the second sub-network only, using the training data. In this way, the machine-learning model may be trained to learn the representations of the chemical structures using the pretraining data, taking advantage of the inevitably larger pretraining data set. Specifically, it is preferred that the pretraining data set comprises more data records than the training data set. More specifically, we mean that the pretraining data set includes more different chemical structures than the training data set. Herein, when it is stated that the first sub-network is “frozen”, this means that during the subsequent training step, the weights which have been learned during pretraining of the first sub-network are fixed, so that only the weights of the nodes in the second sub-network vary during training of that sub-network.

The in vitro measurements may comprise one or more of kinetic solubility, permeability across the artificial membrane (PAMPA), hepatocyte in vitro clearance (Clint) and microsomal Clint. The inventors have shown that some particular combination of in vitro parameters and pharmacokinetic parameters produce particularly good results:
When the pharmacokinetic parameter is CLp, and the in vitro measurements comprise one or more of microsomal Clint and hepatocyte Clint, it has been shown that the model is more precise, and correlation is improved.
When the pharmacokinetic is F and the in vitro measurements comprise one or more of kinetic solubility and PAMPA, it has been shown that overprediction bias was reduced and correlation was improved.

The above disclosure relates to the generation of a machine-learning model for prediction of one or more pharmacokinetic parameters. A second aspect of the present invention provides a computer-implemented method of predicting a pharmacokinetic property of a pharmaceutical compound having a chemical structure, the computer-implemented method comprising: receiving input data encoding the chemical structure of the compound; applying a machine-learning model generated using the computer-implemented method of the first aspect of the invention to the input data, the machine-learning model configured to generate an output comprising a predicted value of one or more pharmacokinetic parameters of the pharmaceutical compound, wherein the machine-learning model has been trained using training data comprising a plurality of records, each record comprising data encoding the chemical structure of a compound, and the values of one or more pharmacokinetic parameters obtained from measurements taken from a human or animal subject after administration of the compound to the human or animal subject; and outputting the one or more predicted values of the pharmacokinetic parameters of the pharmaceutical compound. Naturally, where relevant, optional features set out above in respect of the first aspect of the invention may be applied equally well to the second aspect of the invention. In particular, the preprocessing steps which are applied to the training data during training may also be applied to the input data to ensure that the machine-learning model is applied to data having the same form as the data on which it was trained. Similarly, the input data may take the same form in terms of being e.g. a molecular fingerprint, or a graph structure, as set out previously in respect of the first aspect of the invention.

Additional steps may be carried out after the values of the pharmacokinetic parameters have been predicted. The purpose of such steps is to improve the human intelligibility or explainability of the results, thus allowing the results to be presented in a more ergonomic manner. As such, a computer-implemented method of selecting one or more pharmaceutical compounds during a drug design process may comprise calculating one or more explainability values indicating a relationship between the chemical structure and the predicted pharmacokinetic property, and displaying the predicted pharmacokinetic property and a representation of the one or more explainability values. Thus, the additional steps may assist or guide the user in selecting a pharmaceutical compound for further investigation.

This may be achieved in the context of the present invention using Shapley Additive Explanations (SHAP). The SHAP algorithm is based on game theoretical Shapley values, and is used to provide post hoc explanations for individual predictions, regardless of the machine-learning model which is used. Accordingly, the computer-implemented method may further comprise calculating an importance value (or a SHAP value) for each feature of the input data using a SHAP algorithm. The one or more explainability values may be calculated from the importance values. Each explainability value may indicate a relationship between a respective atom or bond within the chemical structure and the one or more predicted pharmacokinetic properties. In this case, calculating an explainability value for a given atom or bond may comprise combining or summing the importance values associated with the respective atom or bond. The importance values associated with the respective atom or bond may correspond to importance values of features of the input data for which the atom or bond is considered present. In this way, the importance values may be related to the chemical structure. Thus, each explainability value may indicate an extent to which the respective atom or bond affects the one or more predicted pharmacokinetic properties.

When the input data comprises a molecular fingerprint, the molecular fingerprint may comprise an array of values such as a vector, such as a vector of bits. Each feature of the input data may correspond to a given component of the vector, such as a given bit. The importance values associated with a respective atom or bond may correspond to the importance values of the vector components for which the atom or bond is considered present. For example, an atom or bond may be considered present in a certain vector component if the atom or bond corresponds to the central atom or bond represented by this vector component. For example, when the vector is a vector of bits, each “1” bit may represent a central atom, and the environment around this central atom. An atom or bond may be considered present within a certain vector component if the atom or bond is within a predetermined radius around the central atom represented by this vector component. A radius in this context may refer to the distance away from the central atom, measured in a total number of bonds and/or atoms. The predetermined radius may be 2 for example.

In other words, the computer-implemented method may further comprise, calculating importance values (or SHAP values) for each input feature, such as each bit of a Morgan2 fingerprint. Then, to relate the importance values with the chemical structures, each atom and bond may be assigned a explainability value based on the sum of the importance values for each Morgan2 fingerprint in which it is present. Specifically, an atom or bond may be considered present in a certain molecular fingerprint bit if it is within the central atom of this bit, or a radius of 2 around it.

In an additional step, molecules' atoms and/or bonds may be coloured based on the assigned explainability values. The atoms and/or bonds with the highest explainability values may be highlighted. For example, a predetermined number of atoms and/or bonds with the highest explainability values may be highlighted, or the atoms and/or bonds with explainability values over a predetermined threshold may be highlighted. In this way, a clinician viewing the results is able to glean which features of the molecule give rise to the observed properties. This explainability increases the intelligibility of the results.

Improving the human intelligibility or explainability of the results, may additionally or alternatively be achieved in the context of the present invention using a dimensionality reduction technique, for example a UMAP dimensionality reduction technique. The dimensionality reduction technique may be used to visualise the domain of applicability of the machine-learning model, hence aiding a clinician in selecting pharmaceutical compounds for drug discovery. Accordingly, the computer-implemented method may further comprise applying a dimensionality reduction algorithm to the data encoding the chemical structure of the pharmaceutical compounds in the training data to obtain reduced-dimensionality training data points, each reduced-dimensionality training data point representing a chemical structure of a pharmaceutical compound in the training data, and applying the dimensionality reduction algorithm to the input data encoding the chemical structure of the pharmaceutical compound to obtain a reduced-dimensionality input data point, the reduced-dimensionality input data point representing a chemical structure of the pharmaceutical compound in the input data. The reduced-dimensionality input data point may be referred to as the explainability value. That is, the dimensionality reduction algorithm may reduce the dimensionality of the data which it is applied to. The dimensionality reduction algorithm may be a UMAP dimensionality reduction algorithm, for example. The reduced-dimensionality training data points and/or the reduced-dimensionality input data point may be two-dimensional or three-dimensional. As such, the computer-implemented method may further comprise displaying a graph which plots the reduced-dimensionality training data points and the reduced-dimensionality input data point. When the dimensionality reduction algorithm is a UMAP dimensionality reduction algorithm, the graph may be referred to as a UMAP graph/plot/visualisation.

Advantageously, a clinician viewing the graph may be able to interpret how accurate the one or more predicted pharmacokinetic properties corresponding to the pharmaceutical compound represented by the input data are likely to be. For example, if the graph displays the reduced-dimensionality input data point within a predetermined distance of a cluster of reduced-dimensionality training data points, a clinician may understand that the accuracy of the prediction is likely to be sufficiently high. If the graph displays the prediction point over a predetermined distance away from any clusters of training data points, a clinician may understand that the accuracy of the prediction is likely to be too low. Such a method may therefore aid the clinician in selecting a pharmaceutical compound for drug discovery. A clinician may select only pharmaceutical compounds for drug discovery which have desirable predicted pharmacokinetic properties that have been predicted with sufficiently high accuracy. The dimensionality reduction technique may be carried out before the prediction of the one or more pharmacokinetic properties of a pharmaceutical compound. The computer-implemented method may comprise calculating a distance between the reduced-dimensionality input data point and each reduced-dimensionality training data point. The computer-implemented method may only carry out the prediction of the one or more pharmacokinetic properties if there are a predetermined number of reduced-dimensionality training data points within a predetermined distance of the reduced-dimensionality input training data point. The computer-implemented method may only carry out the prediction of the one or more pharmacokinetic properties if the smallest calculated distance is below a predetermined threshold. Advantageously then, pharmaceutical compounds may only be selected for further investigation in the drug discovery context if their pharmacokinetic properties can be predicted with sufficiently high accuracy.

As explained earlier in this application, an important application for computer-implemented methods of the present invention is in drug design, because it can enable drug design teams to make decisions about the likely pharmacokinetic properties of drugs (i.e. pharmaceutical compounds) at an earlier stage in the process. This may ultimately save effort and resources in obtaining in vivo measurements of pharmacokinetic properties of a synthesized compound which are shown, using the present invention, to be undesirable. Accordingly, a third aspect of the invention may provide a method (preferably a computer-implemented method) of selecting one or more pharmaceutical compounds during a drug design process, the method comprising: receiving input data comprising data encoding the chemical structures of one or more pharmaceutical compounds of interest during a drug design process; predicting one or more pharmacokinetic properties of each of the pharmaceutical compounds using the computer-implemented method of the second aspect of the invention; and selecting one or more of the pharmaceutical compounds for further investigation based on the one or more predicted values of the pharmacokinetic parameters of each compound. In some cases, the selection may take place automatically based on the values of the pharmacokinetic parameters.

Naturally, the basis on which the selection is made is specific to the particular context, including the mechanism of action, e.g. agonist, antagonist, positive allosteric modulator etc. The target interaction is also important (e.g. orthosteric v. allosteric, reversible v. irreversible), as are potency, relationship between pharmacokinetic and pharmacodynamics (e.g. AUC-driver, Cₘₐₓ-driven³, Cₘᵢₙ-driven⁴), and many other factors. ³ Cₘₐₓ-maximum concentration⁴ Cₘᵢₙ-minimum concentration

However, as a general rule, for an orally administered pharmaceutical compound, it is generally desirable to aim for high oral bioavailability, low plasma clearance, moderate Vss, and high AUCpo/AUCiv. Compounds meeting this criteria are likely to be successful across a variety of pharmacokinetic, pharmacodynamic, and safety studies. Accordingly, in some cases, selection of one or more pharmaceutical compounds for further investigation based on the one or more predicted values of the pharmacokinetic parameters of the compound may comprise one, a subset or all of the following:
Selecting one or more pharmaceutical compounds having an oral bioavailability which is greater than or equal to a predetermined minimum oral bioavailability. In some contexts, a suitable maximum oral bioavailability threshold may be 50%, but it will be appreciated that this should be determined by a clinician depending on the context. For example, the computer-implemented method may further comprise receiving an input specifying the value of the predetermined minimum oral bioavailability threshold.
Selecting one or more pharmaceutical compounds having a plasma clearance which is less than or equal to a predetermined maximum plasma clearance threshold. In some contexts, a suitable maximum plasma clearance threshold may be 30 ml/min/kg for a mouse, 20 ml/min/kg for a rat, and 5 ml/min/kg for a human. Again, it will be appreciated that this should be determined by a clinician depending on the context. For example, the computer-implemented method may further comprise receiving an input specifying the value of the predetermined maximum plasma clearance threshold.
Selecting one or more pharmaceutical compounds having a Vss value between a predetermined minimum Vss threshold and a predetermined maximum Vss threshold (where “between” may be considered to mean that the values of the minimum and maximum threshold are permitted). In some contexts, a suitable minimum Vss threshold is 1 l/kg, and a suitable maximum Vss threshold is 5 l/kg, but yet again it will be appreciated that this should be determined by a clinician depending on the context. For example, the computer-implemented method may further comprise receiving an input specifying the value of the predetermined minimum Vss threshold and/or the value of the predetermined maximum Vss threshold.
Selecting one or more pharmaceutical compounds having an AUC (used to refer, here, to either AUCiv, or AUCpo) greater than or equal to a predetermined minimum AUC threshold. In some contexts, a suitable minimum AUC threshold may be 500 ng·h/mL/mg/kg, but once more it will be appreciated that this should be determined by a clinician depending on the context. For example, the computer-implemented method may further comprise receiving an input specifying the value of the predetermined minimum AUC threshold.

Further aspects of the invention may comprise a system comprising a processor configured to execute the computer-implemented method of either the first or second aspect of the invention. Another aspect of the invention may provide a drug design decision support system configured to execute the computer-implemented method of the third aspect of the invention.

Additional aspects of the invention may provide a computer program comprising instructions which, when executed by a computer or a processor thereof, cause it to execute the computer-implemented method of the first, second, or third aspect of the invention. Further additional aspects of the invention may provide a computer-readable medium storing the computer program of the previous aspect of the invention.

The invention includes the combination of the aspects and preferred features described except where such a combination is clearly impermissible or expressly avoided.

### BRIEF DESCRIPTION OF THE DRAWINGS

Embodiments of the present invention will now be described with reference to the accompanying drawings, in which:

FIG. 1 is an overview of an approach to leverage historical mouse and rat PK data, as well as complementary in vitro ADME data, towards computational predictions of nonclinical PK.

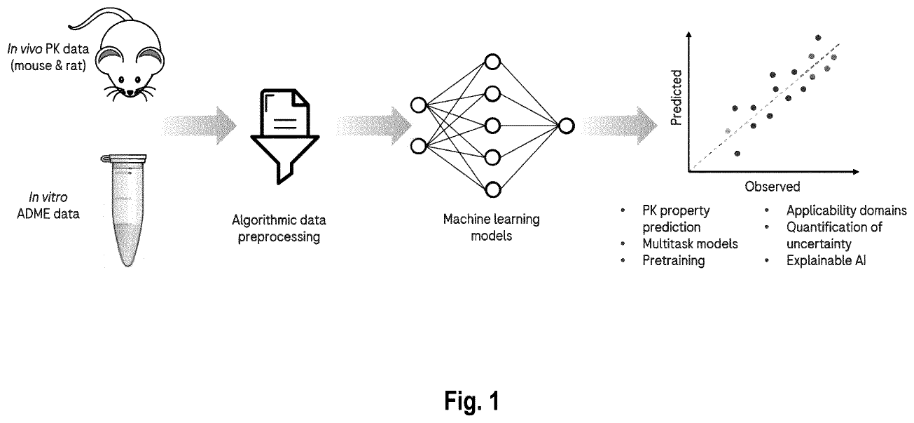

FIG. 2 shows general prediction performance of 10 Single-Task ML model, expressed as AAFE, for mouse and rat PK parameters, including AUCiv/po (h*ng/ml/mg/kg), Vss (L/kg), CLp (mL/min/kg), and F (%). Additional prediction performance metrics are presented in Table 2. Error bars indicate the standard deviation (S.D.) of the random 5-fold cross-validation. Experimental variability is approximated as the experimental average absolute fold error (AAFEₑₓₚ) between the median values and corresponding individual replicates (see Material and Methods for additional details).

FIG. 3 shows correlations between observed and predicted mouse CLp (mL/min/kg), Vss (L/kg), and F (%) using AutoGluon (A), ChemProp (B), and GP (C) ML methods and 5-fold random split cross-validation. For additional performance metrics of these methods, see Table 2. Symbol shading represents unique cycles of the 5-fold cross-validation process. Green, yellow, and red lines represent the unity, 2-fold, and 3-fold errors, respectively. Blue line is the linear fit of the observed correlation. For CLp and Vss, observed data cut-offs are a consequence of winsorization.

FIG. 4 demonstrates the influence of pretraining with in vitro data on prediction performance of ChemProp neural network models for oral F (panels A-C) and CLp (panels D-F). Error bars are S.D. of the 5-fold cross-validation process using random data splitting. Models of oral F were pretrained with either PAMPA permeability (n=121,000) or kinetic aqueous solubility (n=171,000). Models of CLp were pretrained with either microsomal Clint (n=71,000-90,500), hepatocyte Clint (n=9,400-13,500), or CLp extrapolated from both microsomes and hepatocytes using the Well-stirred liver model (n≅80,000-100,000). For the large ChemProp model, the depth and number of neurons per layer in the message passing part have been increased to 10 and 400, respectively.

FIG. 5 shows the influence of splitting approaches towards precision (A), bias (B), and correlation (C) performance of AutoGluon models.

FIG. 6 shows quantification of GP predicted variances for the CLp Mouse test set. (a) confidence plot depicting the AAFE for consecutive subsets of the test data where entries with a high variance are removed first (upper line). The lower line corresponds to an optimal ranking based on the actual error. (b) UMAP embedding of the test set molecules, shaded according to their GP variances. (c) GP variance histograms for the test set data, split into all compounds that are part of a cluster (left side) and singletons (right side). Vertical lines depict the mean of each distribution. Clustering based on the hdbscan package. (d) Same as (c), but shading based on the cluster assigned by hdbscan. Singletons depicted in gray.

FIG. 7 shows the Application of SHAP post-hoc explainability method using AutoGluon predictions of rat Vss values.

FIG. 8 shows Distribution of molecular descriptors for the pre-processed in vivo PK dataset. (A) Parent molecular weight. (B) Total polar surface area. (C) AlogP. (D) # of H bond donors.

FIG. 9 shows distribution of physicochemical properties of molecules from the pre-processed in vivo PK dataset. (A) logD at pH 7.4. (B) kinetic solubility. (C) PAMPA permeability.

FIG. 10 shows distribution of in vitro ADME properties of molecules from the pre-processed in vivo PK dataset. (A) Mouse microsomal clearance. (B) Rat microsomal clearance. (C) Mouse intrinsic hepatocyte clearance. (D) Rat intrinsic hepatocyte clearance.

FIG. 11 shows distribution of log-transformed dose-normalized AUC values of molecules from the pre-processed in vivo PK dataset (units for AUC: h*ng/ml/mg/kg). (A) log (AUC/dose) iv mouse. (B) log (AUC/dose) po mouse. (C) log (AUC/dose) iv rat. (D) log (AUC/dose) po rat.

FIG. 12 shows the distribution of log-transformed iv volume of distribution and iv clearance of molecules from the pre-processed in vivo PK dataset (units for Vdss: L/kg, units for Clp: ml/min/kg). (A) Vdss iv mouse. (B) Clp iv mouse (C) Vdss iv rat. (D) Clp iv rat.

FIG. 13 shows the distribution of untransformed and logit-transformed po bioavailability molecules from the pre-processed in vivo PK dataset. (A) F po mouse. (B) logit F po mouse. (C) F po rat. (D) logit F po rat.

FIG. 14 shows correlations between observed mouse and rat CLp (A), Vss (B), F (C), AUCiv (D), AUCpo I.

FIG. 15 shows correlations of observed pharmacokinetic parameters within species for mouse and rat pharmacokinetic studies.

FIG. 16 shows analysis of replicate measurements for CLp, Vss, and oral F observed in mouse and rat pharmacokinetic measurements.

FIG. 17 is a schematic diagram of a system which may be used in the present invention.

FIG. 18 is a flowchart illustrating a computer-implemented method of training a machine-learning model for prediction of pharmacokinetic parameters.

FIG. 19 is a flowchart illustrating a computer-implemented method of training a machine-learning model for prediction of pharmacokinetic parameters.

FIG. 20 is a flowchart illustrating a computer-implemented method of using a machine-learning model for prediction of pharmacokinetic parameters.

FIG. 21 is a flowchart illustrating a computer-implemented method of using a machine-learning model for prediction of pharmacokinetic parameters in a drug design process.

### DETAILED DESCRIPTION OF THE DRAWINGS

Aspects and embodiments of the present invention will now be discussed with reference to the accompanying figures. Further aspects and embodiments will be apparent to those skilled in the art. All documents mentioned in this text are incorporated herein by reference.

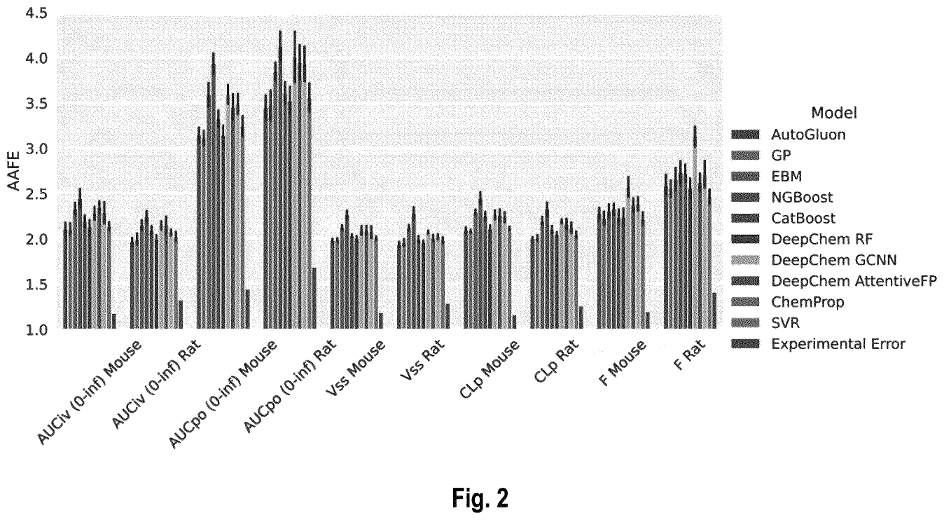

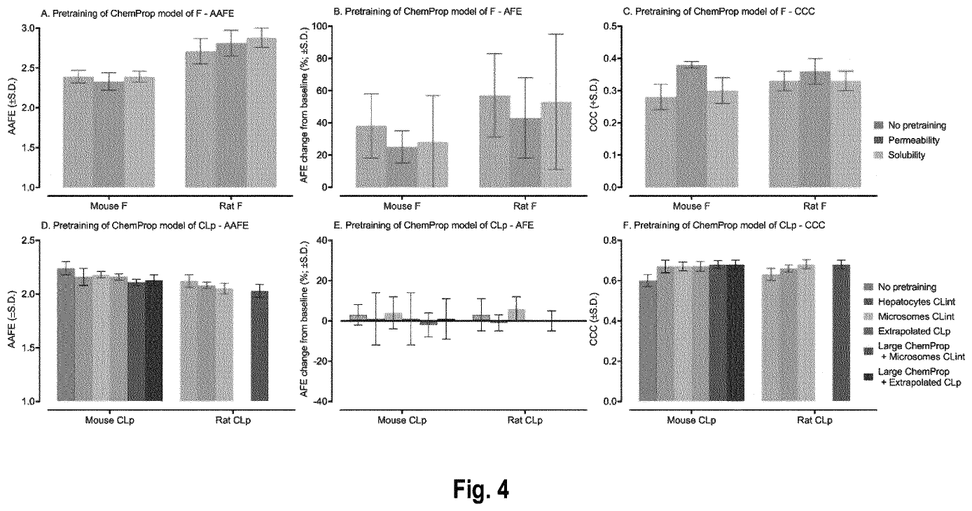

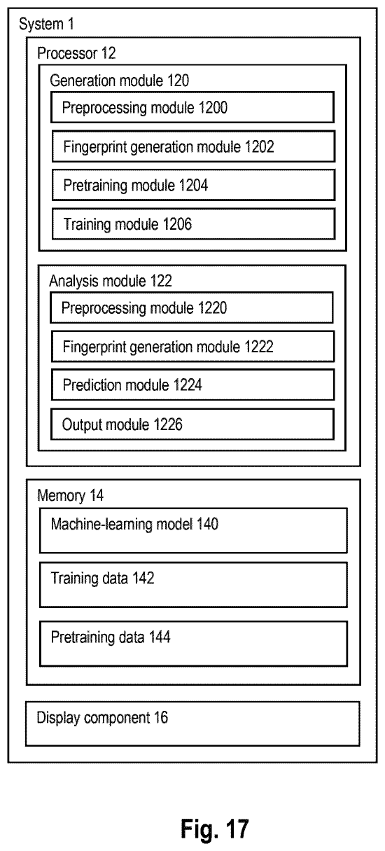

FIG. 17 is a schematic diagram of a system 1 which may be used to execute computer-implemented methods according to the present invention. The system 1 comprises a processor 12, a memory 14, and a display component 16. In FIG. 17, these components are all shown to be part of the same system, but it will be appreciated that the system may be a distributed system in which the various components are located on different pieces of hardware, optionally in different locations. In those cases, the components (e.g. the processor 12, the memory 14, and the display component 16) may be connected via a network (not shown). The network may be a wired network such as a LAN, or WAN, or a wireless network such as a Wi-Fi network, the Internet, or a cellular network. We now discuss the structure of the clinical support system 1 before discussing, with reference to FIGS. 2 and 4, the operations which it is configured to execute. The processor 12 includes a plurality of modules. Herein, the term “module” is used to refer to a functional module which is configured or adapted to execute a particular function. The modules may be implemented in hardware (i.e. they may be separate physical components within a computer), in software (i.e. they may represent separate sections of code, which when executed by processor 12, cause it to perform a particular function), or in a combination of both.

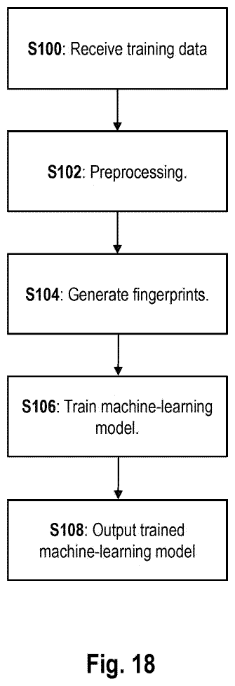

We now discuss the structure of the system 1 in more detail, before discussing its operation in more detail, with references to FIGS. 18 to 21.

The processor 12 includes two modules, a generation module 120 and an analysis module 122. Broadly, the generation module 120 is configured to generate a machine-learning model 140 for prediction of pharmacokinetic properties of one or more pharmaceutical compounds, e.g. by executing a computer-implemented method according to the first aspect of the invention. And, broadly, the analysis module 122 is configured to use the machine-learning model 140 to predict the pharmacokinetic properties of one or more pharmaceutical compounds, e.g. by executing a computer-implemented method according to the second aspect of the invention. The generation module 120 comprises a preprocessing module 1200, a fingerprint generation module 1202, a pretraining module 1204, and a training module 1206. The analysis module 122 comprises a preprocessing module 1220, a fingerprint generation module 1222, a prediction module 1224, and an output module 1226.

It must be emphasized that In some cases, the system 1 may not include e.g. the generation module 120, or the analysis module 122. This is because the machine-learning model 140 may be generated on a separate system from the system on which it is ultimately used. However, in other cases, the machine-learning model may be used on the system on which it was generated. The generation module 120 and the analysis module 122 are shown as part of the same system 1 here for convenience, but it will be appreciated that this absolutely need not be the case.

The memory 14 stores the machine-learning model 140, training data 142, and pretraining data 144. It should be noted that the memory 14 may not necessarily store all of these items at the same time. The memory 14 may comprise a persistent memory and/or a non-persistent (temporary, or buffer) memory. In cases where the generation module 120 and the analysis module 122 are not located on the same system (see previous paragraph), the memory 14 may only be configured to store some subset of the machine-learning model 140, the training data 142, and the pretraining data 144.

The display module 16 may take any typical form such as a monitor, VPU, or the screen of e.g. a smartphone, tablet, laptop computer, desktop computer, or other suitable electronic device. Such display components 16 are ubiquitous in the art and do not require any further explanation.

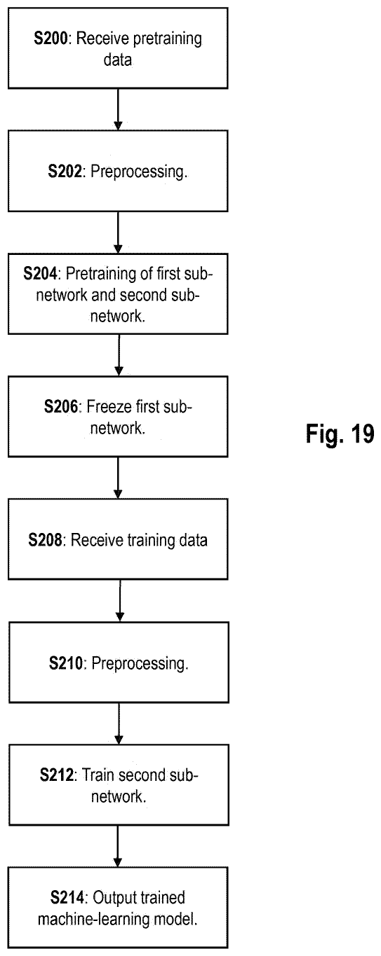

We now explain the function of the generation module 120 using two examples, that of FIG. 18 and FIG. 19 respectively.

FIG. 18 shows an example of a machine-learning model generation process (e.g. according to a computer-implemented method of the first aspect of the invention) which may be used in implementations in which the machine-learning model 140 comprises one or more of a gradient boosting model, an automatic machine-learning model, a Gaussian process regression (GPR) model, a support vector regression (SVR) model, or a random forest model. In a first step S100, training data 142 is received. The training data 142 preferably comprises a plurality of records, each record containing data encoding the chemical structure of a pharmaceutical compound, and the values of one or more pharmacokinetic parameters obtained from measurements taken from a human or animal subject after administration of the compound to the human or animal subject. Next, in step S120, the training data 142 may under preprocessing by the preprocessing module 1200 of the generation module 120.

In one specific implementation, the following preprocessing steps may be carried out: (1) high dose levels may be removed (>100 mg/kg), assuming that higher doses are part of toxicokinetic and safety evaluations, not standard PK measurements; (2) AUCiv and AUCpo values may be extrapolated from zero to infinity were used (0-inf), unless the extrapolation factor was too high (>2-fold), where observed zero to last (0-last) values should be used instead if Tlast>7 h; (3) only measurements in plasma and blood may be included and other matrix tissues were excluded (e.g. brain and liver); (4) only compounds with a molecular weight (MW) between 0 and 1000 Da may be included; (5) observed F values may be limited to range of 0-100% (while values of F>100% are theoretically possible due to saturation of elimination pathways and/or enterohepatic circulation, observed values were winsorized to 100%, assuming that most of these observations are arising from experimental uncertainty); (6) the distribution of results may be normalized, values of CLp, Vss, AUCiv, and AUCpo may be log₁₀ transformed, whereas values of F may be logit-transformed; (7) outliers from multiple measurements may be excluded, when >5 replicate measurements were available, median values and corresponding standard deviations (S.D.) may be found found and individual replicates >2 S.D. were removed; (8) molecular structures may be standardized using Salt remover, sanitize, remove hydrogens, metal disconnector, normalize, reionize, and assign stereochemistry functions from RDKit; (9) compounds with <3 atoms may be excluded; (10) to remove peptidic compounds that display significant secondary structure, hexapeptides may be excluded (via a substructure search with SMILES: NCC(═O)NCC(═O)NCC(═O)NCC(═O)NCC(═O)NCC═O); (11) individual replicate values, per compounds and animal species, may be aggregated by median value; (12) distributions of PK results may be analysed and extreme values >99^{th} percentile and <1^{st} percentile may be winsorized to the 99^{th} and 1^{st} percentile, respectively. Naturally, any subset of these preprocessing techniques may be used without falling outside the scope of the invention.

After preprocessing of the data in step S102, fingerprints are generated using the fingerprint generation module 1202 in step S104. For example, in one implementation, Morgan2 fingerprints folded to 1024 bits may be calculated using RDKit. Optionally, molecular descriptors may also be added to the data. When using the kinds of machine-learning model 140 outlined above, molecular fingerprints are a desirable input because they provide a standardized representation the chemical structure which can be input into machine-learning models 140 which do not generate their own representation.

In step S106, the machine-learning model 140 is trained using the preprocessed data, using the generated molecular fingerprints and the training data 142. The training is performed by the training module 1206. After training, in step S108, the trained machine-learning model 140 is output, and may be stored in memory 14.

FIG. 19 shows an alternative process which may be executed by the generation module 120. The process of FIG. 19 is preferably executed when the machine-learning model 140 is a graph neural network which is configured to learn its own representation of the chemical structures, enabling it to eschew the use of molecular fingerprints. The fact that a graph neural network is able to generate its own representations means that there is capacity for pretraining using the pretraining module 1204. In step S200 of FIG. 19, pretraining data 144 is received by the generation module 120. Similar to the training data 142 (on which more shortly), the pretraining data may comprise a plurality of records, each record containing data encoding the chemical structure of a pharmaceutical compound. The data encoding the chemical structure is preferably in the form of a graph comprising nodes and edges, each node and edge having values associated therewith, the values defining the chemical properties of the atoms and bonds within the molecule. In addition, each record may contain the values of one or more parameters obtained from in vitro measurements, or machine-learning predictions of values to be obtained in in vitro measurements. In step S202, the same preprocessing as was performed in step S102 of FIG. 18 may be performed. The details of the preprocessing will not be repeated here, for conciseness.

In step S204, the pretraining module 1204 pretrains the machine-learning model using the pretraining data 144. As outlined elsewhere in this application, a graph neural network may include a first sub-network which is configured to learn a representation of the data in terms of hidden features of variables, and a second sub-network which is configured to predict the values of the one or more pharmacokinetic parameters based on the learned representation. In step S204, the30ndication30g module 1204 may train the whole network (i.e. the first sub-network and the second sub-network) using the pretraining data 144. In this way, the graph neural network can be trained to generate a representation which is generally effective to predict in vitro measurements, which generally correlate with the in vivo data. After this step, in step S206, the first sub-network may be frozen, i.e. the weights which are used to generate the representation of the chemical structures are fixed. Then, in step S208 training data 142 may be received. The training data 142 takes the same form as for FIG. 18, and in step S210 undergoes the same preprocessing of step S202, and step S102 of FIG. 18. Then, in step S212, the training module 1206 trains the graph neural network using the training data 142. In this step, because the first sub-network is fixed, only the weights of the second sub-network may vary, i.e. the weights which enable the graph neural network to predict the values of the pharmacokinetic parameters based on the representation generated by the first sub-network. After training is complete, the trained machine-learning model is output in step S214, and may be stored in memory 14.

It should be noted that regardless of whether the machine-learning model 140 is trained according to the computer-implemented method of FIG. 18 or FIG. 19, it is able to achieve highly effective results.

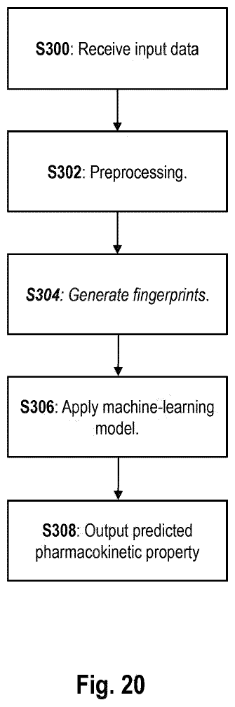

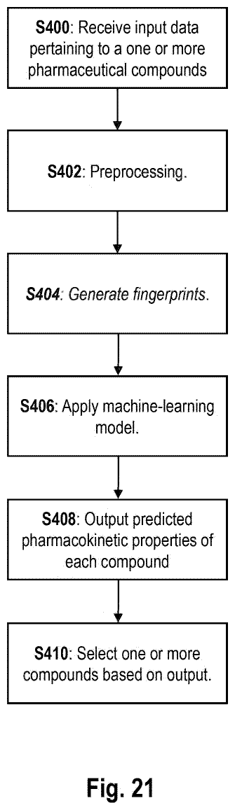

We now turn to FIGS. 20 and 21 which relate to the application of the machine-learning model 140 which may be generated using the methods shown in FIG. 18 or FIG. 19. The method of FIG. 20 is executed by the analysis module 122. In step S300, input data is received by the analysis module 122. The input data preferably encodes the chemical structure of a pharmaceutical compound whose pharmacokinetic properties are to be predicted. In some cases, the input data may be in the form of a graph structure, as outlined earlier, the graph structure comprising nodes and edges, each node and edge having values associated therewith, the values defining the chemical properties of the atoms and bonds within the molecule. In other cases, the chemical structure may be described in an alternative way. In step S302, preprocessing is performed on the input data, in the same way as in e.g. step S102 of FIG. 18, or steps S202 and S210 of FIG. 19. It should be noted, of course, that some of the preprocessing steps are not applicable to data concerning a single chemical structure. These steps are, of course, not performed. In particular, at this stage, the preprocessing module 1220 may standardize the chemical structures by removing salts; sanitization; removal of hydrogen atoms; disconnecting metals; normalization; reionization; and assigning stereochemistry. After preprocessing, in an optional step S304, molecular fingerprints may be generated using fingerprint generation module 1222. It will be appreciated that this is not necessary in implementations in which the machine learning model 140 is a graph neural network (in which case the input data is in the form of a graph structure, and no molecular fingerprint generation is required). In step S306, the machine-learning model 140 is applied by the prediction module 1224, and in step S308 the one or more predicted pharmacokinetic properties are output by output module 1226. The outputs may then be transmitted to display component 16 for display. More specifically, output module 1226 may generate instructions which when executed by display component 16, cause it to display the output of the machine-learning model 140.

FIG. 21 demonstrates a way in which the trained machine-learning model 140 may be used during a drug design process. In step S400, input data pertaining to a one or more pharmaceutical compounds may be received. Then, steps S402 to S406 are performed in respect of each of the pharmaceutical compounds. These steps are analogous to steps S302 to S306 of FIG. 20. In step S408, one or more predictions are output, each corresponding to a respective pharmaceutical compound. Then, in step S410, a selection of one or more pharmaceutical compounds is made based on the outputs.

We now present detailed experimental results, obtained from the application of the present invention.

### EXPERIMENTAL RESULTS

### Materials and Methods

### Pharmacokinetic Dataset

PK properties observed in standard single-dose mouse and rat PK studies were retrieved from the Roche database in May 2022, namely CLp (mL/min/kg), Vss (L/kg), and dose-normalized AUCiv (h*ng/ml/mg/ml) after the IV administration, and F (%) and AUCpo (h*ng/mL/mg/mL) after the PO administration. A majority of PK studies were performed with standard drug formulations, using either a single-compound or a cassette PK design with coadministration of up to four compounds. For IV dosing, a solution of compound in N-methyl-2-pyrrolidone mixed with saline or 2-hydroxypropyl-β-cyclodextrin was commonly used (30:70% v/v; for IV dosing). For PO dosing, the most common formulation was a suspension of test compound with 0.1% dioctyl sulfosuccinate sodium salt and parabens in 1.25% hydroxypropylmethylcellulose (pH 6). All PK properties were calculated based on observed total plasma or blood concentrations (exposures in other tissues were excluded), using noncompartmental PK analysis. The terminal plasma half-life (t½) and mean residence time (MRT) were not directly predicted, as they are composite parameters of CLp and Vss

$$( i . e . , {t}_{1 / 2} = \frac{ln 2 * Vss}{CLp} ; {t}_{1 / 2} = ln 2 * MRT ) .$$

Initial database query retrieved around 10,000 unique compounds tested in 30,000 individual PK studies.

### In Vitro Dataset

In vitro intrinsic metabolic clearance (Clint) was measured in either mouse and rat liver microsomes (μL/min/mg; n=90,500 mouse, n=71,000 rat) or hepatocytes (μL/min/10⁶ cells; n=13,500 mouse; n=9,400 rat) using standard drug discovery assays (Kratochwil et al., 2017). Microsomal and hepatocyte Clint values were also extrapolated to predicted CLp using the Well-stirred liver model

$${CL}_{p} = \frac{{Q}_{h} * {CL}_{int , scaled}}{{Q}_{h} + {CL}_{int , scaled}} ,$$

where Qₕ is the liver blood flow (91 and 60 mL/min/kg for mouse and rat, respectively) and Clint, scaled is the intrinsic clearance of the whole liver, calculated as CL_{int,scaled}=CLᵢₙₜ*scaling factor*liver weight. The scaling factor for microsomes (microsomal recovery per gram of liver) was 45 mg/g liver and for hepatocytes (hepatocellularity) 120 million cells/g liver. Liver weight was 57.7 and 40 g/kg body weight for mouse and rat, respectively. Aqueous solubility (μg/mL) was measured in a phosphate buffer (50 mM, pH 6.5; n=171,000) using a kinetic solubility approach (Alsenz and Kansy, 2007). Passive permeability (cm/s 10⁻⁶) was measured across artificial membranes using the PAMPA approach (n=121,000) (Kansy et al., 2004). In vitro datasets were used for pretraining of DeepChem neural networks.

### Algorithmic Data Preprocessing

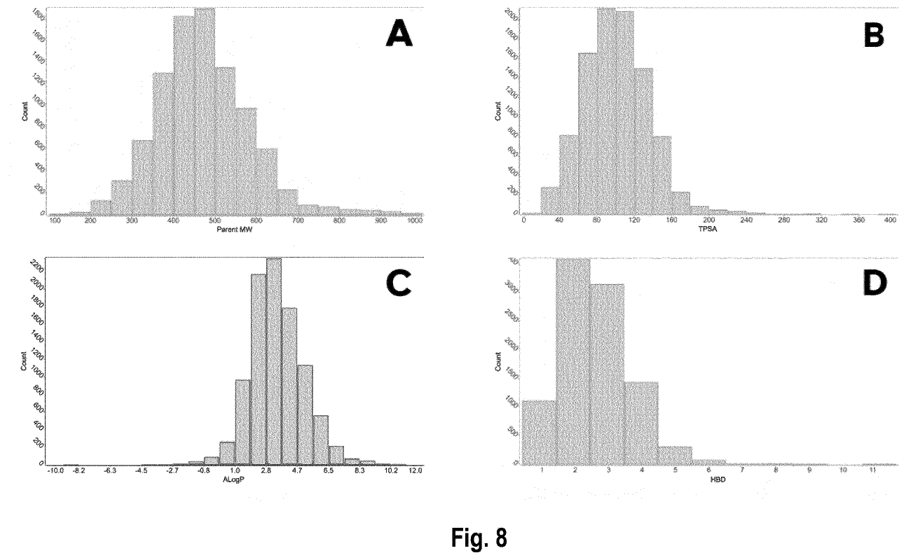

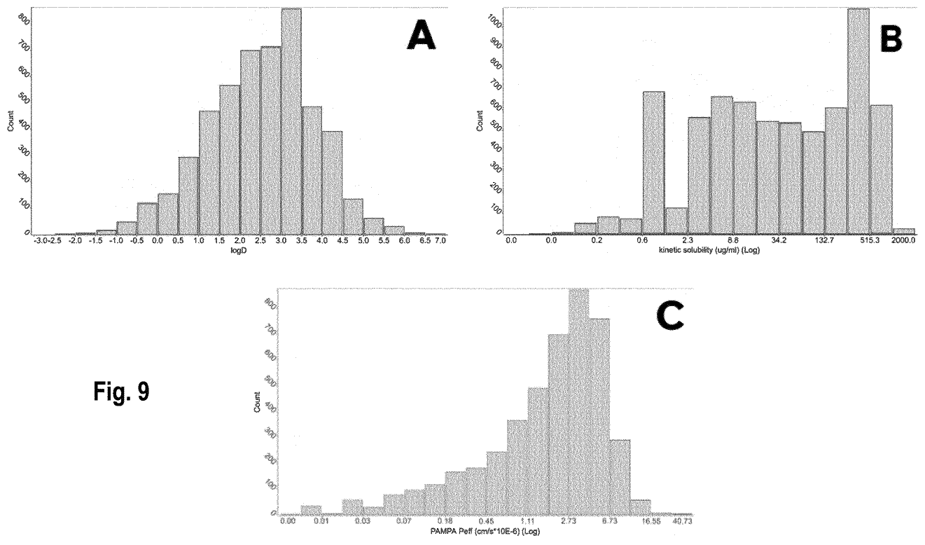

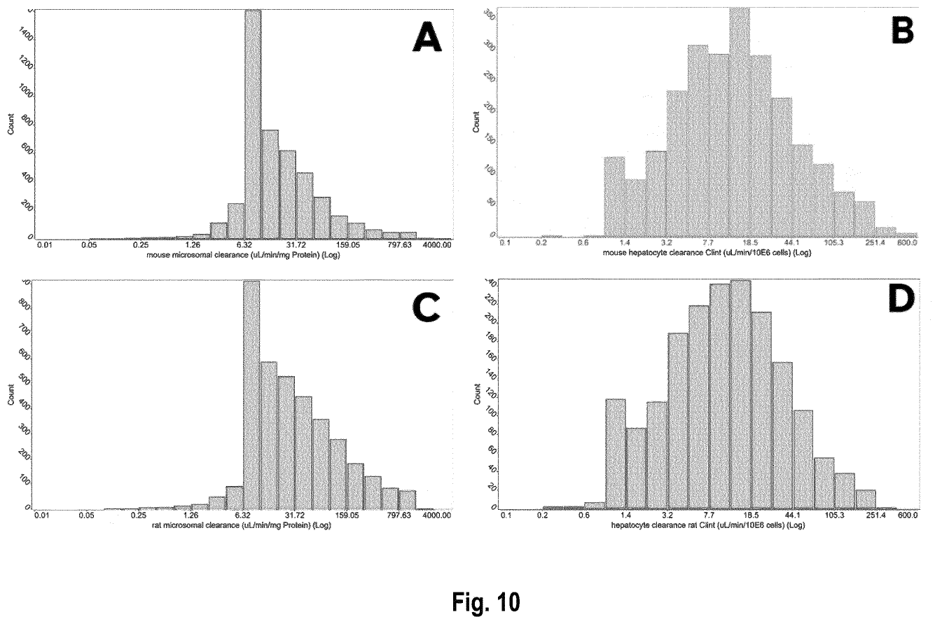

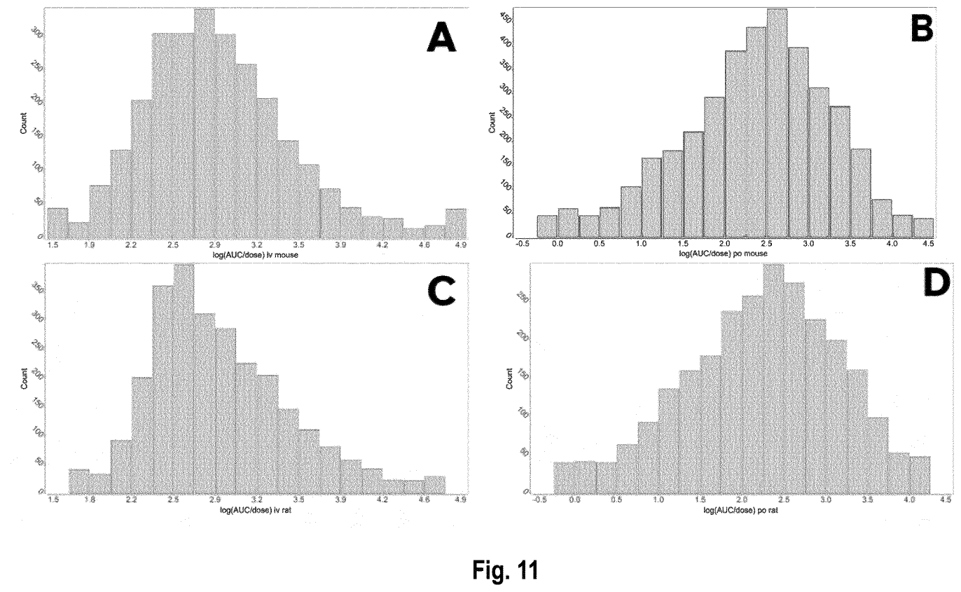

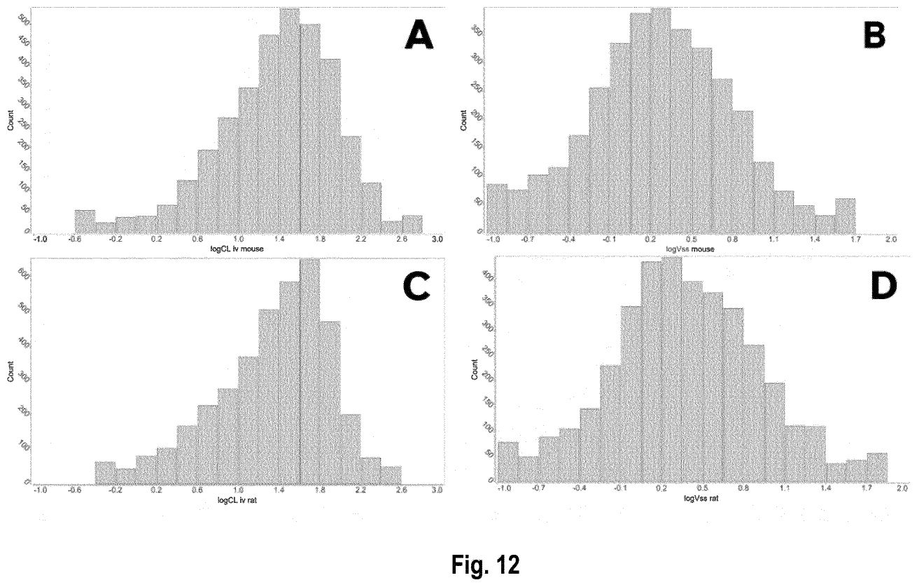

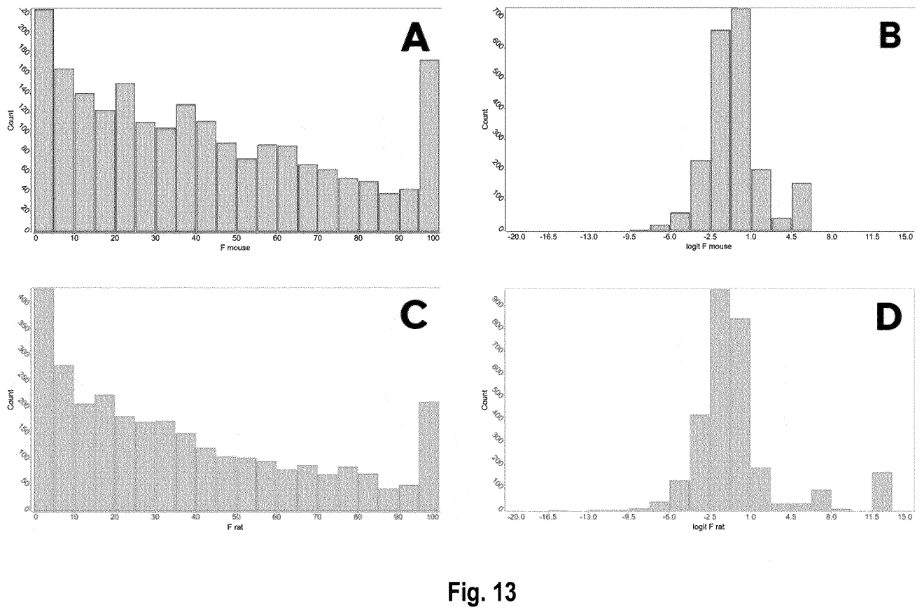

For the in vivo mouse and rat PK data retrieved from the Roche database, following preprocessing steps were applied: (1) high dose levels were removed (>100 mg/kg), assuming that higher doses are part of toxicokinetic and safety evaluations, not standard PK measurements; (2) AUCiv and AUCpo values extrapolated from zero to infinity were used (0-inf), unless the extrapolation factor was too high (>2-fold), where observed zero to last (0-last) values were used instead; (3) only measurements in plasma and blood were included and other matrix tissues were excluded (e.g. brain and liver); (4) only compounds with a molecular weight (MW) between 0 and 1000 Da were included; (5) observed F values were limited to range of 0-100% (while values of F>100% are theoretically possible due to saturation of elimination pathways and/or enterohepatic circulation, observed values were winsorized to 100%, assuming that most of these observations are arising from experimental uncertainty); (6) to normalize the distribution of results, values of CLp, Vss, AUCiv, and AUCpo were log 10-transformed, whereas values of F were logit-transformed (Obrezanova et al., 2022); (7) to exclude outliers from multiple measurements, when >5 replicate measurements were available, median values and corresponding standard deviations (S.D.) were found and individual replicates >2 S.D. were removed; (8) molecular structures were standardized using Salt remover, sanitize, remove hydrogens, metal disconnector, normalize, reionize, and assign stereochemistry functions from RDKit (Landrum 2021); (9) compounds with <3 atoms were excluded; (10) to remove peptide compounds that display significant secondary structure, hexapeptides were excluded (via a substructure search with SMILES: NCC(═O)NCC(═O)NCC(═O)NCC(═O)NCC(═O)NCC═O); (11) individual replicate values, per compounds and animal species, were aggregated by median value; (12) distributions of PK results were analysed and extreme values >99^{th} percentile and <1^{st} percentile were winsorized to the 99^{th} and 1^{st} percentile, respectively. The resulting dataset consisted of 2091-3895 unique chemical structures per PK property (see Table 3 for details). Distributions of key molecular properties and observed PK results are presented in FIGS. 8 to 13. Summary statistics of molecular properties and PK results are presented in Tables 4 and 5, respectively.

### Assessment of Experimental PK Variability

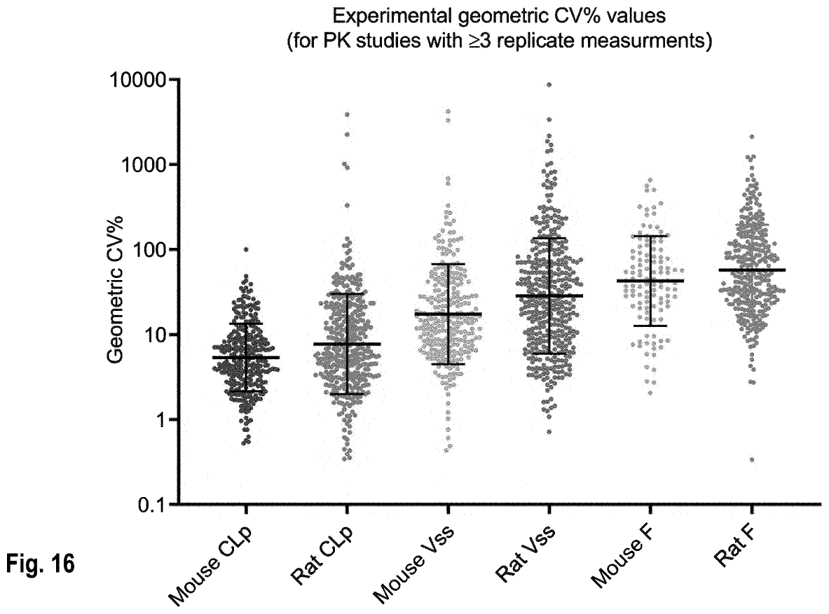

To assess an average experimental variability of our mouse and rat PK results, all studies with 23 replicates were analysed further. Firstly, to assess an average fold error of replicate experiments, we compared individual measurements with the corresponding median for each compound and calculated the fold error between individual measurements and median values. Table 6 reports the geometric mean of observed fold errors for all data available for the specific property and animal species (AAFEₑₓₚ). Secondly, to assess the average precision of replicates, we calculated the average geometric coefficients of variation (% CV) for all data available for the specific property and animal species. This analysis showed that the lowest average % CV were observed for CLp values (5.4 and 7.8% for mouse and rat, respectively). Observed Vss values had average % CV of 17.5 and 28.7%, respectively, and oral F values exhibited the highest variability, with &CV values of 43 and 58%, respectively (FIG. 16).

### Featurization

Morgan2 (corresponding to ECFP4) fingerprints, folded to 1024 bits, and 195 molecular descriptors were calculated by RDKit (Rogers and Hahn, 2010). While a combination of fingerprints and descriptors slightly increased the performance of AutoGluon over fingerprints alone (our best performing model), the35ndicarence was statistically insignificant, and so Morgan2 fingerprints were used for all models except graph neural networks. In addition, as one of the key goals was to provide a model explanation in terms of structural features only, Morgan2 fingerprints provided a framework for this.

### Machine Learning Models

10 Single-Task ML models were used, with default settings. The model selection included boosting models such as CatBoost 0.25.1 (Prokhorenkova et al., 2017), NGBoost 0.3.11 (Duan et al., 2019) and Explainable Boosting Machines (EBM) (Lou et al., 2013), the automated ML model AutoGluon Tabular 0.2.0 (Erickson et al., 2020), Gaussian Process Regression models (GP; with Tanimoto kernel using gpflow 2.2.1) (Williams and Rasmussen, 1995), Support Vector Regression models (SVR; using scikit-learn 0.23.2) (Drucker et al., 1997), Random Forest (Breiman, 2001) used with DeepChem 2.5.0 and graph neural networks such as graph convolutional neural networks (GCNN) (Xie and Grossman, 2018) from DeepChem, Attentive Fingerprints (AttentiveFP) (Xiong et al., 2020) from DeepChem and ChemProp (Yang 2019). In addition to Single-Task models, AutoGluon, ChemProp, and DeepChem AttentiveFP were tested in multi-task mode, with training across all PK properties from both species. ChemProp neural network models of oral F and CLp were used for pretraining tests with in vitro data. All models except for the graph neural networks use Morgan2 fingerprints.

### Assessment of Splitting Approaches

To assess the performance of ML models, experimental results were divided into training and test sets using random, scaffold (Murcko Scaffolds from RDKit), SCINS (scaffold class identification and naming system) (Schuffenhauer et al., 2007), and time splits. For random, scaffold, and SCINS splits, a 5-fold cross-validation process was applied, with 80% and 20% of data in the training and test set, respectively. For the time split, the first reported registration date of the compound was relied upon, and 90 and 10% of data were used for training and testing, respectively (in time domain, the test set corresponded to compounds synthesized between 2017 and 2022). Splitting approaches were tested using AutoGluon, GP, and ChemProp models.

### Explanations of Model Predictions

To explain model predictions in terms of molecular features and their relative importance, SHAPley Additive explanations (SHAP) (Lundberg and Lee, 2017) method was used. The SHAP algorithm is based on the game theoretical Shapely values and is used to provide post-hoc explanations for individual predictions, regardless of the ML model that is used to make those predictions. Firstly, SHAP explanations are calculated as importance values for each input feature, in this case one-bit Morgan2 fingerprints, and a sum of these values results in the final prediction (additive nature of SHAP values). Secondly, to relate SHAP importance values with molecular structures, each atom and bond is assigned a numerical value based on the sum of the importance values for each Morgan2 fingerprint in which it is present. Specifically, an atom or bond is considered present in a certain Morgan2 bit if it is within the central atom of this bit, or a radius of 2 around it. Thirdly, molecules' atoms and bonds are coloured based on the assigned numerical values.

### Evaluation of Model Uncertainties

For models that provide prediction uncertainties, the way in which their values are distributed across the dataset's chemical space was of interest. To distinguish between compounds that belong to a cluster of similar compounds the hdbscan (McInnes et al., 2017) (Moulavi et al., 2013) (McInnes et al., 2017) package was used. Compounds were clustered based on their Tanimoto similarity, with clustering parameters set to min_samples=10 and min_cluster_size=30 (otherwise default settings). Morgan2 fingerprints were used as input features. A visual representation of the chemical space was provided through an embedding with the Uniform Manifold Approximation and Projection (UMAP) (McInnes et al., 2020) package for Python (v. 0.5.3), where the Tanimoto similarity was used as a distance metric.

### Assessment of Model Predictions

Several metrics were used to assess prediction bias, precision (i.e. dispersion), and correlations between observations (obs) and predictions (pred). Bias of predictions was assessed with average fold error (AFE), where AFE values of 0<AFE<1 and AFE>1 indicate underprediction and overprediction bias, respectively.

$$AFE = {10}^{\frac{1}{n} {\sum }_{i = 1}^{n} log ( \frac{pred}{obs} )} \qquad ( 1 )$$

Precision of predictions was assessed with absolute average fold error (AAFE), percentage fold error, root mean squared error (RMSE), and root mean squared logarithmic error (RMSLE). The AAFE, also often called the geometric mean fold error, is the geometric mean of individual absolute fold errors, where value of 1 indicates perfect predictions.

$$AAFE = {10}^{\frac{1}{n} {\sum }_{i = 1}^{n} \vert log ( \frac{pred}{obs} ) \vert} \qquad ( 2 )$$

Percentage fold errors, within 2-, 3-, and 5-fold, were calculated as:

$$freq ( max ( \frac{obs}{pred} , \frac{pred}{obs} ) \qquad ( 3 )$$

The RMSE and RMSLE were calculated as:

$$RMSE = \sqrt{\frac{{\sum }_{i = 1}^{n} {( {pred}_{i} - {obs}_{i} )}^{2}}{n}} \qquad ( 4 )$$

$$RMSLE = \sqrt{\frac{{\sum }_{i = 1}^{n} {( log ( {pred}_{i} ) - log ( {obs}_{i} ) )}^{2}}{n}} \qquad ( 5 )$$

Correlations between observations and predictions were assessed with Pearson's correlation coefficient I, coefficient of determination (R²), and concordance correlation coefficient (CCC).

$$R = \frac{{\sum }_{i = 1}^{n} ( {obs}_{i} - \underline{obs} ) ( {pred}_{i} - \underline{pred} )}{\sqrt{{\sum }_{i = 1}^{n} {( {obs}_{i} - \underline{obs} )}^{2}} \sqrt{{\sum }_{i = 1}^{n} {( {pred}_{i} - \underline{pred} )}^{2}}} \qquad ( 6 )$$

$${R}^{2} = 1 - \frac{{\sum }_{i = 1}^{n} {( {obs}_{i} - {pred}_{i} )}^{2}}{{\sum }_{i = 1}^{n} {( {obs}_{i} - \underline{obs} )}^{2}} \qquad ( 7 )$$

$$CCC = \frac{2 R {σ}_{obs} {σ}_{pred}}{{σ}_{obs}^{2} + {σ}_{pred}^{2} + {( \underline{obs} - \underline{pred} )}^{2}} \qquad ( 8 )$$

Where obs and pred are mean values and σ_{obs} and σ_{pred} are variances of observations and predictions, respectively.

### Results

### Mouse and Rat PK Datasets

The historical dataset of mouse and rat PK studies contains around 10,000 unique compounds tested in 30,000 individual PK studies over a period of 25 years. To prepare the dataset for ML approaches, ed an algorithmic data preprocessing pipeline (see Materials and Methods) was developed, resulting in 9,685 unique structures matched with corresponding aggregated PK properties (Table 3). The number of unique compounds and matched PK results varied per species and property, being the highest for CLp (n=3515-3895) and Vss (n=3448-3871), and lowest for the oral F (n=2091-2949) measurements. Analysis across species shows that mostly different compounds were tested in mouse and rat PK studies, with an average species overlap between 8% (for CLp and Vss) and 24% (for oral F). Properties of test compounds were only partially within a classical drug-like space, with 47% conforming to Lipiniski's guidelines, and a significant number of molecules with high MW (36%>500 Da), high topological polar surface area (TPSA; 8%>150 Å), and high lipophilicity (22% with AlogP>5). In the preprocessed dataset, median values of the key properties included a MW of 464 Da, TPSA of 100 Å², 7 hydrogen bond acceptors (HBA), 2 hydrogen bond donors (HBD), and logD of 2.61 (calculated logP of 3.80) (see Table 4). In terms of ionization state, most compounds were neutral (49%), followed by basic (31%), acidic (16%), and zwitterionic (4%) molecules. Despite a relatively low compound overlap between mouse and rat (8-24%), molecular properties were comparable, with the mouse PK dataset showing a trend towards higher median MW (472 vs. 448 Da), TPSA (104 vs. 94 Å), and logD (2.68 vs. 2.57) values.

Reported PK results spanned a broad range of values (>1000-fold), sometimes approaching or exceeding the lower or upper limits of study design, which values were winsorized on the 1^{st} and 99^{th} percentile of observed distributions. Median values were CLp of 28 mL/min/kg (range 0.26-425), Vss of 2 L/kg (range 0.1-425), F of 32% (range 0 to 100%), dose-normalized AUCiv of 660 ng*h/mL/mg/kg (range 34-78, 625), and dose-normalized AUCpo of 243 ng*h/mL/mg/kg (range 0.67-17, 900) (Table 5). Mouse and rat PK results were similar, with rat showing a trend towards higher median CLp (27 vs. 25 mL/min/kg; 40ndicat. 28 vs. 45% of liver blood flow), higher Vss (1.86 vs. 2.25 L/kg), and lower oral F (36 vs. 29%) (Table 5). Analysis of replicate PK measurements resulted in an overall experimental average absolute fold error (AAFEₑₓₚ) of 1.35, with rat data exhibiting a higher variability compared to mice (AAFEₑₓₚ of 1.43 vs. 1.27) (Table 6). Oral PK parameters (i.e. F and AUCpo; AAFEₑₓₚ of 1.48) were more variable compared to IV properties (CLp, Vss, and AUCiv; AAFEₑₓₚ of 1.27). Lastly, mouse and rat PK results were correlated to a various degree, with Pearson's correlation coefficients of 0.83 for AUCiv, 0.62 for Vss, 0.57 for AUCpo, 0.54 for F, and only 0.33 for CLp.

### Performance of Single-Task ML Models

To assess the feasibility of PK predictions at the drug-design stage, 10 Single-Task ML models were first tested towards predictions of mouse and rat PK properties, including CLp, Vss, AUCiv, AUCpo, and F. Model selection included automated ML models (AutoGluon), boosting models (EBM, NGBoost, CatBoost), Bayesian models (GP), random forest (RF), support vector machine (SVM), and graph neural networks (DeepChem GCNN, AttentiveFP, and ChemProp). An overview of prediction performance, expressed as AAFE and estimated using a 5-fold random split cross-validation, is presented in FIG. 2. Additional performance metrics for selected mouse models (AutoGluon, GP, and ChemProp) are presented in Table 2, including a breakdown across individual models, PK properties, and performance metrics. Additional results for all Single-Task models are presented in Table 7.

Average prediction performance, across all Single-Task ML models, PK parameters, and species showed a good precision (AAFE of 2.49; RMSLE of 0.52), low bias (AFE of 1.11), and acceptable correlation (R2 log 10 of 0.36; CCC of 0.54) (FIG. 2). Model prediction error was higher compared to observed experimental errors of replicate PK measurements (AAFEₑₓₚ of 1.35 vs. model AAFE of 2.49), indicating that further improvements may be possible. Comparing between mouse and rat PK models, average prediction performance was similar (AAFE of 2.45 vs. 2.53; RMSLE of 0.51 vs. 0.53; AFE of 1.09 vs. 1.13; R2 log 10 of 0.36 vs. 0.37; CCC of 0.54 vs. 0.55). However, a comparison between IV (Vss, CLp, AUCiv) and PO (F, AUCpo) PK properties showed that an average prediction performance was notably better for IV properties (AAFE of 2.12 vs. 3.04; RMSLE of 0.43 vs 0.65; AFE of 1.00 vs. 1.27; R2 log 10 of 0.44 vs. 0.26; CCC of 0.61 vs. 0.44).

Comparing between 10 individual Single-Task ML models, a generally similar prediction performance has been observed, with average key performance metrics within a relatively narrow range (AAFE 2.35-2.71; RMSLE 0.49-0.56; AFE 1.06-1.15; R2 log 10 0.28-0.43; CCC 0.37-0.60) (FIG. 2, Table 2, Table 7).

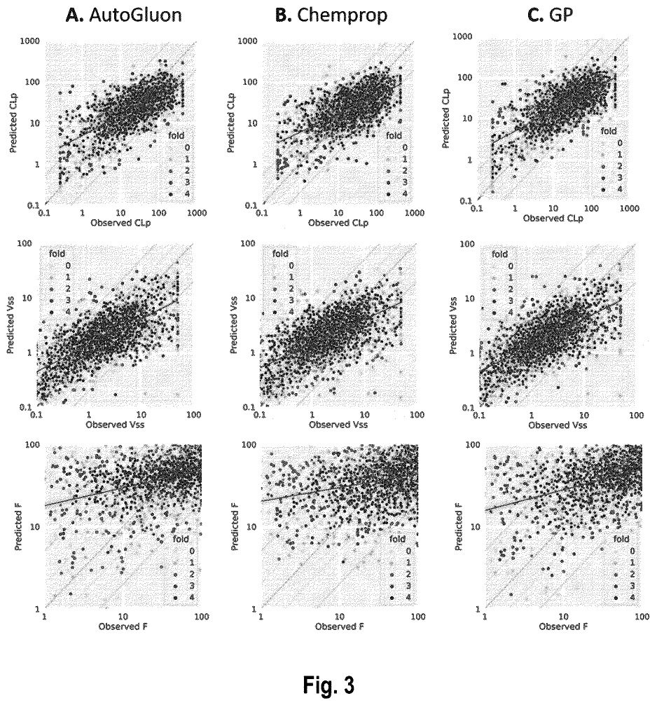

Despite similarities, some trends were observed, namely that AutoGluon, GP, SVR, DeepChem, and ChemProp may offer advantages over EBM, CatBoost, and NGBoost models (Table 2 and Table 7). Taking into account all performance metrics, AutoGluon and GP were likely the highest performing models, offering good precision (equal AAFEs of 2.35; RMSLE of 0.47 and 0.49), low bias (AFEs of 1.12 and 1.11), and good correlation between observed and predicted values (R2 log 10 of 0.42 and 0.43; CCC of 0.59 and 0.60). ChemProp neural network was an example of a middle-performing model (AAFE of 2.53; RMSLE of 0.53; AFE of 1.11; R2 log 10 of 0.35). Correlation plots for mouse CLp, Vss, and F, generated with AutoGluon, GP, and ChemProp models, are shown in FIG. 3. Conversely, NGBoost was the worst performing model (AAFE of 2.71; RMSLE of 0.56; AFE of 1.15; R2 log 10 of 0.28; CCC of 0.37). Notably, for all tested Single-Task models, prediction bias was low for most of the PK properties, including CLp, Vss, AUCiv, and AUCpo (AFE of 0.99-1.04), but models of F showed a tendency towards overpredictions (AFE of 1.49). Compared to the training dataset, AutoGluon and GP showed a significant drop in precision (AAFE drop of >40%) and correlation (CCC drop of >44%) on the test set. On the other hand, when moving from training to test dataset, ChemProp exhibited only a modest reduction in precision (AAFE drop of 18%) and correlation (CCC drop of 27%), indicating that ChemProp may offer advantages in terms of reduced overfitting and prediction generalization.

### Performance of Multi-Task ML Models

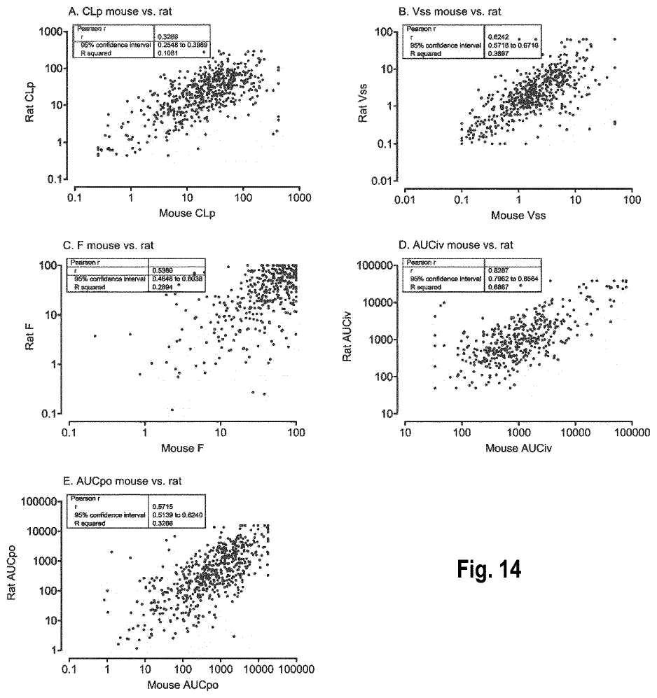

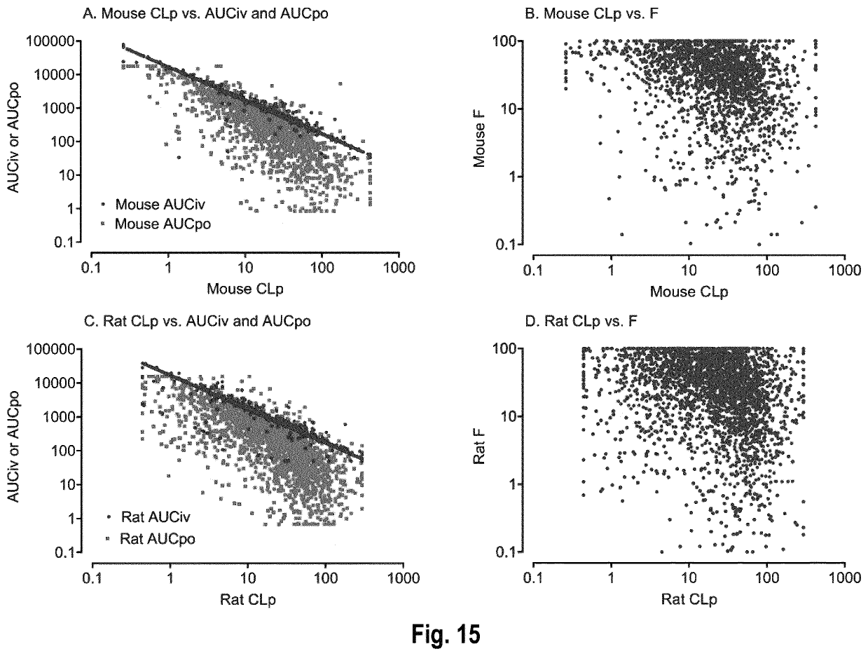

As (1) individual PK properties are often correlated (e.g. a decrease of CLp increases AUCs and F; FIG. 15) and (2) mouse and rat PK results are similar for many chemotypes (FIG. 14), it was hypothesized that Multi-Task ML models may show increased performance over Single-Task models. To test this hypothesis, we used AutoGluon, ChemProp, and DeepChem AttentiveFP models in both Single- and Multi-Task mode. Our Multi-Task approach included model training across all PK properties from both species.

Comparing between Single- and Multi-Task models, no significant differences were observed (Table 5), with all Multi-Task models performing similarly to corresponding Single-Task counterparts. For AutoGluon, the Multi-Task approach showed similar precision and correlation to the Single-Task models (AAFE of 2.42 vs. 2.36; RMSLE of 0.50 vs 0.51; CCC of 0.58 vs. 0.57), but the overprediction bias was slightly reduced (AFE 1.07 vs. 1.12), especially for the oral F (AFE of 1.60 vs. 1.20). Results for ChemProp and DeepChem AttentiveFP were comparable to AutoGluon (Table 5).

### Performance of Pretraining Approaches

As performance of neural networks increases with large datasets and our in vivo datasets are limited in terms of chemical diversity we hypothesized that pretraining of networks with relevant in vitro data may be beneficial for model performance. To test this hypothesis, we have assessed the performance of ChemProp neural networks towards predictions of F and CLp, both in the absence and presence of pretraining with in vitro data. For predictions of F, we have tested pretraining with kinetic solubility (n=171,000) or permeability across the artificial membrane (PAMPA; n=121,000), two molecular properties known to play a major role in oral absorption. For predictions of CLp, we have tested pretraining with either microsomal Clint (n=71,000-90, 500), hepatocyte Clint (n=9,400-13,500), or a combination of both in vitro datasets presented as extrapolated CLp. Results are presented in FIG. 4 and Table 6.

Pretraining of neural networks with in vitro solubility or permeability data showed mixed outcomes towards predictions of F (FIG. 4A). Solubility pretraining showed a negligible effect, with all performance metrics similar to models without pretraining. Permeability pretraining showed a similar precision to non-pretrained models (AAFE of 2.57 vs. 2.55; RMSLE of 0.59 vs. 0.59), but reduced overprediction bias (AFE of 1.34 vs. 1.48) and improved correlation (R2 log 10 of 0.13 vs. 0.10; CCC of 0.37 vs. 0.30).

Pretraining of neural networks with in vitro clearance data showed a modest, but consistent trend towards improved predictions of CLp (FIG. 4B). As an example, pretraining on microsomal Clint modestly improved model precision (AAFE of 2.12 vs. 2.18, RMSLE of 0.43 vs. 0.41) and correlation (R2 log 10 of 0.44 vs. 0.41; CCC of 0.68 vs. 0.62). Encouraged by these results, we have also tried increasing the size of the neural network, namely the number of hidden layers In the message passing from 300 to 400 and its depth from 3 to 10. This enlarged and pretrained neural network showed further performance increase over the non-pretrained model (AAFE of 2.07 vs. 2.18; RMSLE of 0.43 vs. 0.45; AFE of 1.00 vs. 1.05; R2 (log 10) of 0.48 vs. 0.41; CCC of 0.68 vs. 0.62).

### Influence of Data Splitting Approaches on Prediction Performance

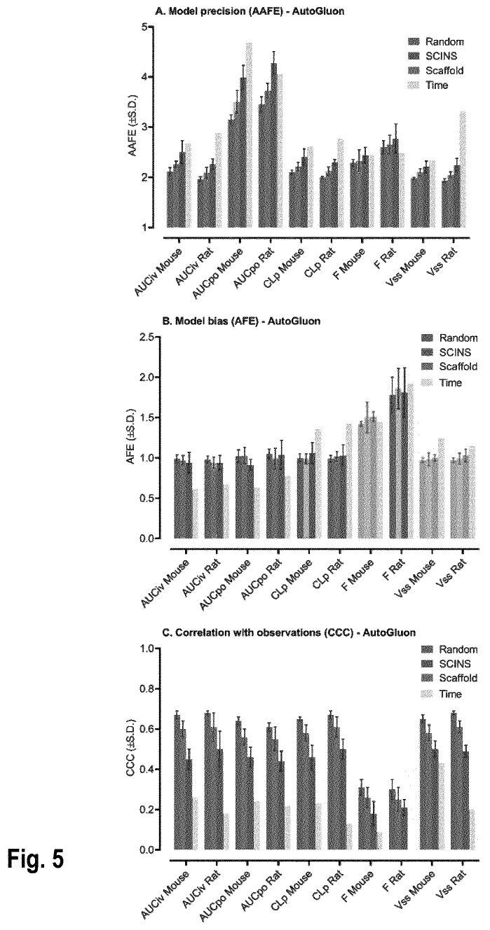

To assess the real-world predictive performance of our ML models, we tested random, scaffold, SCINS, and time splits with Single-Task AutoGluon, GP, and ChemProp models. Whereas random, scaffold, and SCINS splits were evaluated with 80-20% 5-fold cross validation approach, the time split was evaluated by using 10% of the most recent compounds as a test set (corresponding to the last 5 years of research). Using AutoGluon models as a representative example, FIG. 5 highlights typical changes in AAFE (A), AFE (B), and CCC (C) across different splitting techniques. Results obtained with GP and ChemProp models were similar to AutoGluon and are presented in Table 8. In general, averaging over all models, properties, and species, a random data split showed the best overall model performance, with good precision (AAFE of 2.41; RMSLE of 0.51), low bias (AFE of 1.11), and good correlation (R2 log 10 of 0.41; CCC of 0.58). The scaffold split displayed a lower average performance, reflected in lower precision (AAFE of 2.56; RMSLE of 0.53) and poorer correlation (R2 log 10 of 0.33; CCC of 0.51). The results of SCINS split showed an additional performance decrease (AAFE of 2.78; RMSLE of 0.57; R2 log 10 of 0.23; CCC of 0.43). Lastly, the time split showed the lowest performance, with further precision decrease (AAFE of 2.95; RMSLE of 0.62) and low correlation between predictions and observations (R2 log 10 of 0.07; CCC of 0.25).

### Evaluation of Model Uncertainties and Applicability Domains

When a drug design team proposes a new compound, a model's prediction uncertainty can be as valuable as the actual prediction. This is closely related to the question of applicability domains: For a sensible model we expect accurate predictions and low prediction uncertainties when a data point is within a known chemical space. In contrast, compounds from an unknown chemical space should have larger prediction errors and associated uncertainties. Here we demonstrate how the issue of realistic applicability domains can be quantified using uncertainty estimation with GP models and assessed through a combination of clustering and dimensionality reduction techniques. The following results are discussed for the CLp Mouse endpoint.

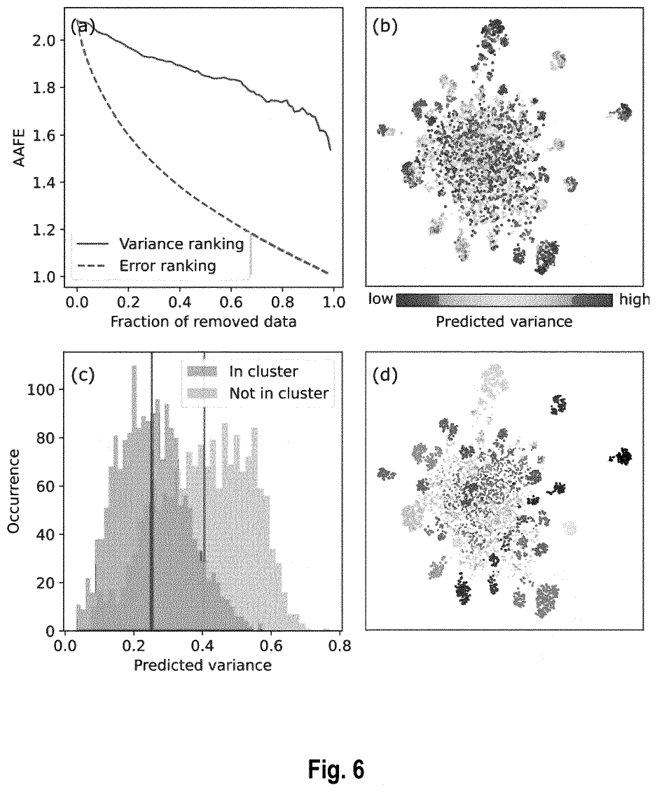

To quantify whether a model's uncertainty estimates are sensible, a number of methods have been proposed (Scalia et al., 2020). Confidence plots (also called sparsification plots) are a common tool (Aodha et al., 2012; Ilg et al., 2018). Here the assumption is that predictions with high uncertainties correspond to large errors. Thus, by sorting a set of predictions by their uncertainties and removing n-% of entries we expect that the overall prediction error of the subset will decrease. This is shown in FIG. 6a for our GP model. The x-axis represents the relative number of points removed from the test set. For example, at x=0.2, 20% of the predictions with the highest uncertainties are removed. The calculated AAFE for each subset is depicted on the y-axis. When sorting by the variance predicted by our GP model, a mostly monotonic decrease in AAFE can be observed, which is the expected behavior. The gray line represents a confidence curve for an ideal ranking, which is given by the prediction error.

Returning to the notion of chemical similarity, we apply an additional approach for uncertainty quantification based on clustering. To that end, we use hdbscan (McInnes et al., 2017) (Moulavi et al., 2013), (McInnes et al., 2017)), to cluster all compounds in our test set based on their Tanimoto similarity. We hypothesize that a sensible model assigns higher uncertainties to compounds that are not part of any cluster. This can be verified in FIG. 6c, depicting GP variance histograms for test set compounds that were part of a cluster (blue) as well as all singletons (orange), with respective mean variances of 0.25 and 0.40 plotted as solid vertical lines. For a visual representation of the test set, UMAP (McInnes et al., 2018) embeddings of each compound's Morgan2 fingerprint were created. Coloring the embedded compounds by their predicted variances in FIG. 6b as well as their cluster label in FIG. 6d gives a visual confirmation of our hypothesis.

### Explanations of Model Predictions

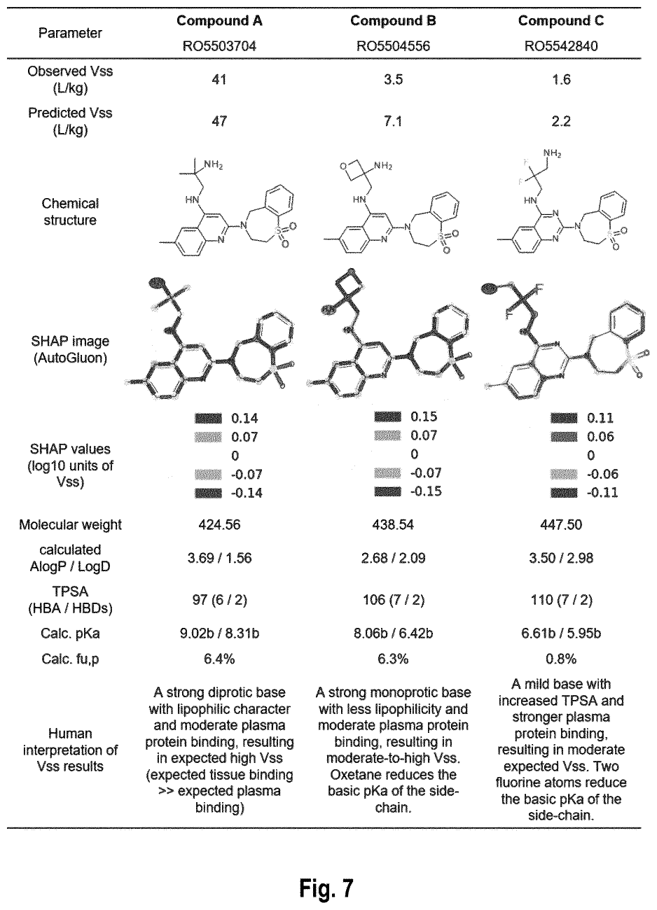

To assess the feasibility and usability of model explanations, we tested the post-hoc SHAP values on predictions of mouse and rat Vss, using AutoGluon and GP models. An example of outcome is presented in FIG. 7, where we interpret the AutoGluon predictions of rat Vss values for a matched set of three compounds from the same chemotype. From a human perspective, the Vss is governed by an interplay of compounds' nonspecific binding to tissues and plasma proteins. If nonspecific binding to tissues is relatively stronger, the resulting Vss tends to be higher (e.g. >3 L/kg). Conversely, if compounds preferentially bind to plasma proteins, the resulting Vss tends to be low or moderate (e.g. ≲2 L/kg). In terms of molecular properties, binding to tissues and plasma proteins is predominantly governed by physicochemical features, mainly lipophilicity (logP and logD), polarity (e.g. TPSA), and ionization constants (i.e. acid and base pKa values).

Compounds in FIG. 7 share most of the structural features, including benzothiazepine and quinoline rings, as well as the pendant sidechain with two basic amines (Zheng et al., 2018). The compound A is strongly basic (calc. basic pKa of 9.02 and 8.31), lipophilic, and less polar, suggesting a high Vss. The compound B is less basic (calc. basic pka of 6.08 and 6.42), less lipophilic, and more polar, suggesting a moderate Vss. Lastly, the compound C is the least basic (calc. basic pKa of 6.61 and 5.95) and the most polar, influenced by two electronegative fluorine atoms in the sidechain and additional nitrogen in the ring (quinoline-quinazoline), suggesting the lowest Vss value. Indeed, these human predictions were confirmed by observed rat Vss values (41, 3.5, and 1.6 L/kg) and captured well by AutoGluon predictions (47, 7.1, and 2.2 L/kg). Similar to the human interpretation, the calculated SHAP values indicate that the pendant sidechain plays a major role in Vss outcome, with oxetane in compound B, as well as fluorine atoms and additional ring nitrogen in compound C, having a strong negative contribution on Vss. Conversely, strongly basic amines in compound A have a positive Vss contribution. Further, SHAP values suggest that lipophilic hydrocarbons (e.g. phenyl rings) have a positive Vss contribution for both compounds, also in line with common human interpretation that unsubstituted hydrocarbons increase lipophilicity. Taken together, these results suggest that SHAP values may aid human understanding of ML predictions. It should be noted, however, that predictions of Vss are likely simpler than predictions of other PK properties such as F or CLp, which may be governed by a complex interplay of molecular properties and physiological systems including enzymes and transporters. As a consequence, while our results offer an initial proof-of-concept, strong conclusions cannot be made and significant further work is needed to tackle the explanations of complex PK properties across a broad range of chemotypes.

### Discussion

After compound synthesis many high-quality ADME assays and integration approaches (e.g. IVIVE and PBPK) are available to predict both nonclinical and clinical PK outcomes. At the drug design stage, however, predictive tools are scarce and many teams rely on rule-of-thumb drug-likeness guidelines (Lipinski, 2004b) and, more recently, ML predictions of individual ADME properties (Göller et al., 2020; Broccatelli et al., 2022). While approaches towards fully computational PK predictions are emerging (see Table 1), many existing studies are based on a small number of compounds and lack human interpretability, the key quality needed for real-life adoption. We focused our attention on this specific challenge, namely the predictions of PK from chemical structure alone, in absence of associated experimental data. In addition to prediction accuracy, we emphasized human interpretability of results by providing insights into prediction uncertainty, applicability domains, and explanations of contributing molecular features.

Our exploratory dataset included mouse and rat PK studies performed at Roche in the last 25 years, consisting of 9,685 unique structures matched with corresponding aggregated PK properties (Table 3). To our knowledge, this is one of the biggest nonclinical PK datasets described thus far, with rat data alone matching recent publications (Schneckener et al., 2019b; Kosugi and Hosea, 2020; Obrezanova et al., 2022) and exceeding the number of publicly reported clinical PK results (Iwata et al., 2021b; Lombardo et al., 2021b; Miljković et al., 2021). Compared to the well-known Lombardo dataset of human PK results (Lombardo et al., 2018b), our rodent dataset showed a significantly higher MW (median of 464 vs. 371 Da), lipophilicity (median calc. logP of 3.8 vs. 2.0; logD of 2.61 vs. 0.7), and polarity (TPSA of 100 vs. 89 Å), with up to 53% of the molecules beyond the classical Lipinski drug-like space (Lipinski, 2004b). Compared to a recently published rat PK dataset (Obrezanova et al., 2022), our compounds appear similarly lipophilic (median logD of 48ndicat. 2.6 vs. 2.3). As our dataset represented an exploratory drug discovery space tested in two rodent species (mouse and rat with overlap of 9%), and observed PK properties spanned a broad dynamic range (median property range of 700-fold; Table 5), we hypothesized that this PK dataset offers a possibility for a proof-of-concept study for applicability of computational PK at the drug-design stage.

After developing an algorithmic pre-processing pipeline, ten Single-Task ML models were built for CLp, Vss, F, AUCiv, and AUCpo (FIGS. 2 and 3; Table 2, Table 7). The best performing models, like AutoGluon and GP, offered a considerable real-life performance across PK properties, with average AAFE values ranging from 2.35 to 2.98, corresponding to 64-74% of predictions within 3-fold from observations. Data splitting played an important role in model performance (random>scaffold>SCINS>time), but even the most demanding SCINS and time splits offered a usable model performance, especially for CLp (AAFE 2.51; 70% within 3-fold) and Vss (AAFE of 2.53; 68% within 3-fold) (FIG. 5). Performance drop of the time split may be explained by the significant evolution of our chemical space in the last five years, exemplified by large increases of MW and TPSA (average MW of 535 vs. 463 Da; average TPSA of 115 vs. 99). From the perspective of PK properties, model performance was notably better for IV properties (CLp, Vss, AUCiv; AAFE of 2.08) compared to PO properties (F and AUCpo; AAFE of 2.96). This result is aligned with recent publications (Schneckener et al., 2019b; Obrezanova et al., 2022) and may be explained by additional variability and complexity of oral PK studies, where compounds' solid form, applied dose, and formulation play a major role in final outcome. Compared to recently developed computational models based on comparable datasets, our ML models showed similar (Murad et al., 2020b; Kosugi and Hosea, 2021b; Lombardo et al., 2021b; Miljković et al., 2021; Obrezanova et al., 2022), improved (Schneckener et al., 2019b; Iwata et al., 2021b), or decreased (Wang et al., 2019a) performance. For example, if focusing on the drug-design stage results (i.e. in absence of in vitro data), Obrezanova et al. showed RMSLE values of 0.35, 0.31, and 0.57 for predictions of rat CLp, Vss, and oral F, respectively (Obrezanova et al., 2022). This result is comparable to our AutoGluon and GP average RMSLE values of 0.42, 0.39, and 0.57, rat CLp, Vss, and oral F, respectively.

From the perspective of ML methodology, we observed that many Single-Task ML models offered performance in a similar range (FIG. 2; Table 2; Table 7), likely limited by the size, quality, and chemical diversity of existing PK datasets. Dataset limitations, in terms of compound number and diversity, may also influence Multi-Task approaches, all of which showed comparable performance to Single-Task models (Table 8), with exception of improved overprediction prediction bias and correlation for oral F. This observation is in accordance with findings from Obrezanova et al. who report overall equivalent performance between Single-Task and Multi-Task models for most PK parameters (Obrezanova et al., 2022). Middling performance of neural networks (ChemProp, AttentiveFP, and DeepChem GCNN), consistently inferior to AutoGluon and GP, offers additional evidence for the dataset limit hypothesis. To increase the dataset size and diversity for graph neural networks, we tested a number of pretraining approaches with relevant in vitro data (Ye et al., 2019), focusing on predictions of CLp and F. Even if observed effects were modest and statistically insignificant (FIG. 4), pretrained ChemProp models of CLp offered consistent improvements of precision, bias, and correlation. Similarly, for ChemProp models of F, pretraining on solubility or permeability improved correlations and reduced bias. While these results demonstrate the potential of pretraining approaches, further research is needed to find optimal pretraining strategies for multivariate PK properties, like oral F, where an interplay of solubility, permeability, CLp governs final outcomes.

One may wonder if observed real-life prediction performance is good enough for ML models to be used by drug design teams. While a direct answer to this question is difficult, a comparison with existing experimental approaches to predict PK may offer a starting point. For example, metabolism-driven CLp is often predicted from in vitro hepatocyte CLint and plasma protein binding (fu, p), resulting in AAFE values of 2-3 [e.g., 2.05-2.53 (Naga et al., 2022), 2.10-3.10 (Kratochwil et al., 2017), 2.0-3.0 (Umehara et al., 2020)]. Additionally, predictions of CLp from experimental data tend to suffer from either under- or over-prediction bias, often emphasized by a human decision on if and how to account for the free drug hypothesis (Poulin and Haddad, 2021). Compared to these published results, our ML models of CLp offer a comparable prediction performance (AAFEs of 2.05-2.66) and low bias (AFEs of 1.00-1.36), in absence of any experimental input. Similarly, Vss can be predicted based on experimental fu, p, fu, tissue, logD, and pka values, often leading to AAFE values of 1.5-3.0, depending on the ionization class (Mathew et al., 2021). Compared to these prediction errors, our best models of mouse and rat Vss offered similar performance (AAFE of 1.96-2.83). Lastly, oral F can be predicted via an integration of various in vitro results, including solubility, permeability, metabolic Clint, and transporter liabilities, often within PBPK models. If experimental data are used, reported prediction success offers AAFE values of 1.85-3.50 (Paixão et al., 2012; Naga et al., 2022), comparable to our best models (AAFE of 2.35-2.61). Based on these comparisons, developed ML models likely offer a sufficient level of prediction accuracy to aid drug design decisions, for example to facilitate a selection between numerous design ideas or de-prioritize compounds with poor predicted outcomes. Importantly, they leverage decades of historical data into useful predictions about the future, taking into account overall complexity of PK outcomes.

In relation to model performance, when a new design compound is suggested, one can ask whether the model will be predictive for this specific new design (Mervin et al., 2020). To answer this question, average prediction performance estimated in post-hoc cross-validation is insufficient, as prediction uncertainty may be different for each individual new compound, likely driven by their chemical similarity to previously tested compounds. Instead, a compound-specific quantification of uncertainty is needed, to provide a user feedback a priori, at the moment of prediction. Reliable quantification of uncertainty is needed for both decision making and identification of compounds outside the prediction domain, where predictions are infeasible and generation of in vitro and in vivo data may be justified. Using GP models to quantify prediction variance (Costabal et al., 2019), we observed a significant connection between the former and the presence of related compounds forming clusters of similar chemotypes (FIG. 6). These results indicate that GP probability distribution offers a useful way to quantify model uncertainty at the drug design stage. Similar results were recently reported for GP rat CLp model, using a ranking-based confidence curve (Obrezanova et al., 2022).

Historically, due to “black box” predictions, ML models were perceived as unsuitable for drug design, where quantitative and causal understanding of structure-activity relationships are the key to progress. Recently, methodology of explainable AI improved (Jiménez-Luna et al., 2020), leading to several successful examples in computational predictions of both pharmacophores (Harren et al., 2021) and ADME properties (Jiménez-Luna et al., 2021). Explainable AI was sparsely used in the field of PK predictions, with only Iwata et al. reporting the use of Deep Tensor to highlight partial structures contributing to human CLp (Iwata et al., 2021b). Our tests of post-hoc SHAP values (Lundberg and Lee, 2017) on mouse and rat Vss predictions (FIG. 7) indicated that useful structural explanations are possible, corresponding to human concepts of functional groups, lipophilicity, polarity, and amine basicity. While this approach offers promise for drug design teams, further research is needed to apply it for complex properties like CLp or oral F, governed by multiple factors. One may argue that there is no simple structure-activity relationship for complex properties like CLp, governed by an interplay of metabolism, transport, and nonspecific binding to plasma proteins and tissues. This complexity argument is self-evidently true, but it is also unusable and ill-structured for drug design teams, which need to propose a discrete modification on the molecule in hope of addressing overwhelming complexity. Therefore, a computational approach to highlight key contributing structural features may help, especially for complex chemotypes beyond the classical drug-like space.

In conclusion, our results suggest that computational predictions of PK are feasible at the drug design stage, especially if several convergent methodologies are used to tackle predictions, uncertainty, applicability, and explanations at once. Presented direct and hybrid approaches are also complementary to computational predictions of ADME properties (and corresponding PBPK integrations), working together to illuminate different sides of complexity.

### General Statements

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value.

When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### REFERENCES

Alsenz J, and Kansy M (2007) High throughput solubility measurement in drug discovery and development. Adv Drug Deliver Rev 59:546-567.
Amo E M del, Ghemtio L, Xhaard H, Yliperttula M, Urtti A, and Kidron H (2013) Applying Linear and Non-Linear Methods for Parallel Prediction of Volume of Distribution and Fraction of Unbound Drug. Plos One 8:e74758.
Aodha O M, Humayun A, Pollefeys M, and Brostow G J (2012) Learning a Confidence Measure for Optical Flow. Ieee T Pattern Anal 35:1107-1120.
Bender A, Jenkins J L, Scheiber J, Sukuru S C K, Glick M, and Davies J W (2009) How Similar Are Similarity Searching Methods? A Principal Component Analysis of Molecular Descriptor Space. J Chem Inf Model 49:108-119.
Breiman L (2001) Random Forests. Mach Learn 45:5-32.
Broccatelli F, Trager R, Reutlinger M, Karypis G, and Li M (2022) Benchmarking Accuracy and Generalizability of Four Graph Neural Networks Using Large In Vitro ADME Datasets from Different Chemical Spaces. Mol Inform 2100321.
Costabal F S, Matsuno K, Yao J, Perdikaris P, and Kuhl E (2019) Machine learning in drug development: Characterizing the effect of 30 drugs on the QT interval using Gaussian process regression, sensitivity analysis, and uncertainty quantification. Comput Method Appl M 348:313-333.
Cumming J G, Davis A M, Muresan S, Haeberlein M, and Chen H (2013) Chemical predictive modelling to improve compound quality. Nat Rev Drug Discov 12:948-962.
Danishuddin, Kumar V, Faheem M, and Lee K W (2021) A decade of machine learning-based predictive models for human pharmacokinetics: advances and challenges. Drug Discov Today, doi: 10.1016/j.drudis.2021.09.013.
Drucker H, Burges CJC, Kaufman L, and Smola A J (1997) Support Vector Regression Machines. Advances in Neural Information Processing Systems.
Duan T, Avati A, Ding D Y, Thai K K, Basu S, Ng A Y, and Schuler A (2019) NGBoost: Natural Gradient Boosting for Probabilistic Prediction. Arxiv.
Erickson N, Mueller J, Shirkov A, Zhang H, Larroy P, Li M, and Smola A (2020) AutoGluon-Tabular: Robust and Accurate AutoML for Structured Data. Arxiv.
Fagerholm U, Hellberg S, and Spjuth O (2021) Advances in Predictions of Oral Bioavailability of Candidate Drugs in Man with New Machine Learning Methodology. Molecules 26:2572.
Falcón-Cano G, Molina C, and Cabrera-Pérez M A (2020) ADME Prediction with KNIME: Development and Validation of a Publicly Available Workflow for the Prediction of Human Oral Bioavailability. J Chem Inf Model 60:2660-2667.
Freitas A A, Limbu K, and Ghafourian T (2015) Predicting volume of distribution with decision tree-based regression methods using predicted tissue: plasma partition coefficients. J Cheminformatics 7:6.
Göller A H, Kuhnke L, Montanari F, Bonin A, Schneckener S, Laak A ter, Wichard J, Lobell M, and Hillisch A (2020) Bayer's in silico ADMET platform: a journey of machine learning over the past two decades. Drug Discov Today 25:1702-1709.
Gombar V K, and Hall S D (2013) Quantitative Structure-Activity Relationship Models of Clinical Pharmacokinetics: Clearance and Volume of Distribution. J Chem Inf Model 53:948-957.
Harren T, Matter H, Hessler G, Rarey M, and Grebner C (2021) Interpretation of Structure-Activity Relationships in Real-World Drug Design Data Sets Using Explainable Artificial Intelligence. J Chem Inf Model, doi: 10.1021/acs.jcim.1c01263.
Hosea N A, and Jones H M (2013) Predicting Pharmacokinetic Profiles Using in Silico Derived Parameters. Mol Pharmaceut 10:1207-1215.
Ilg E, Çiçek Ö, Galesso S, Klein A, Makansi O, Hutter F, and Brox T (2018) Uncertainty Estimates and Multi-Hypotheses Networks for Optical Flow. Arxiv.
Iwata H, Matsuo T, Mamada H, Motomura T, Matsushita M, Fujiwara T, Kazuya M, and Handa K (2021a) Prediction of Total Drug Clearance in Humans Using Animal Data: Proposal of a Multimodal Learning Method Based on Deep Learning. J Pharm Sci 110:1834-1841.
Iwata H, Matsuo T, Mamada H, Motomura T, Matsushita M, Fujiwara T, Kazuya M, and Handa K (2021b) Prediction of Total Drug Clearance in Humans Using Animal Data: Proposal of a Multimodal Learning Method Based on Deep Learning. J Pharm Sci 110:1834-1841.
Jiménez-Luna J, Grisoni F, and Schneider G (2020) Drug discovery with explainable artificial intelligence. Nat Mach Intell 2:573-584.
Jiménez-Luna J, Skalic M, Weskamp N, and Schneider G (2021) Coloring Molecules with Explainable Artificial Intelligence for Preclinical Relevance Assessment. J Chem Inf Model 61:1083-1094.
Kamiya Y, Handa K, Miura T, Yanagi M, Shigeta K, Hina S, Shimizu M, Kitajima M, Shono F, Funatsu K, and Yamazaki H (2021) In Silico Prediction of Input Parameters for Simplified Physiologically Based Pharmacokinetic Models for Estimating Plasma, Liver, and Kidney Exposures in Rats after Oral Doses of 246 Disparate Chemicals. Chem Res Toxicol 34:507-513.
Kansy M, Avdeef A, and Fischer H (2004) Advances in screening for membrane permeability: high-resolution PAMPA for medicinal chemists. Drug Discov Today Technologies 1:349-355.
Kosugi Y, and Hosea N (2020) Direct Comparison of Total Clearance Prediction: Computational Machine Learning Model versus Bottom-Up Approach Using In Vitro Assay. Mol Pharmaceut 17:2299-2309.
Kosugi Y, and Hosea N (2021a) Prediction of Oral Pharmacokinetics Using a Combination of In Silico Descriptors and In Vitro ADME Properties. Mol Pharmaceut 18:1071-1079.
Kosugi Y, and Hosea N (2021b) Prediction of Oral Pharmacokinetics Using a Combination of In Silico Descriptors and In Vitro ADME Properties. Mol Pharmaceut 18:1071-1079.
Kratochwil N A, Meille C, Fowler S, Klammers F, Ekiciler A, Molitor B, Simon S, Walter I, McGinnis C, Walther J, Leonard B, Triyatni M, Javanbakht H, Funk C, Schuler F, Lave T, and Parrott N J (2017) Metabolic Profiling of Human Long-Term Liver Models and Hepatic Clearance Predictions from In Vitro Data Using Nonlinear Mixed-Effects Modeling. Aaps J 19:534-550.
Kumar R, Sharma A, and Varadwaj P K (2011) A prediction model for oral bioavailability of drugs using physicochemical properties by support vector machine. J Nat Sci Biology Medicine 2:168-173.
Lipinski C A (2004a) Lead- and drug-like compounds: the rule-of-five revolution. Drug Discov Today Technologies 1:337-341.
Lipinski C A (2004b) Lead- and drug-like compounds: the rule-of-five revolution. Drug Discov Today Technologies 1:337-341.
Lombardo F, Bentzien J, Berellini G, and Muegge I (2021a) In Silico Models of Human P K Parameters. Prediction of Volume of Distribution Using an Extensive Data Set and a Reduced Number of Parameters. J Pharm Sci 110:500-509.
Lombardo F, Bentzien J, Berellini G, and Muegge I (2021b) In Silico Models of Human P K Parameters. Prediction of Volume of Distribution Using an Extensive Data Set and a Reduced Number of Parameters. J Pharm Sci 110:500-509.
Lombardo F, Berellini G, and Obach R S (2018a) TREND ANALYSIS OF A DATABASE OF INTRAVENOUS PHARMACOKINETIC PARAMETERS IN HUMANS FOR 1352 DRUG COMPOUNDS. Drug Metab Dispos 46: dmd. 118.082966.
Lombardo F, Berellini G, and Obach R S (2018b) TREND ANALYSIS OF A DATABASE OF INTRAVENOUS PHARMACOKINETIC PARAMETERS IN HUMANS FOR 1352 DRUG COMPOUNDS. Drug Metab Dispos 46: dmd. 118.082966.
Lou Y, Caruana R, Gehrke J, and Hooker G (2013) Accurate intelligible models with pairwise interactions. Proc 19^{th} Acm Sigkdd Int Conf Knowl Discov Data Min 623-631.
Lundberg S, and Lee S-I (2017) A Unified Approach to Interpreting Model Predictions. Arxiv.
Madden J C, Enoch S J, Paini A, and Cronin MTD (2020) A Review of In Silico Tools as Alternatives to Animal Testing: Principles, Resources and Applications. Altern Laboratory Animals 48:146-172.
Mathew S, Tess D, Burchett W, Chang G, Woody N, Keefer C, Orozco C, Lin J, Jordan S, Yamazaki S, Jones R, and Di L (2021) Evaluation of Prediction Accuracy for Volume of Distribution in Rat and Human Using In Vitro, In Vivo, PBPK and QSAR Methods. J Pharm Sci 110:1799-1823.
McInnes L, Healy J, and Astels S (2017) hdbscan: Hierarchical density based clustering. J Open Source Softw 2:205.
McInnes L, Healy J, and Melville J (2020) UMAP: Uniform Manifold Approximation and Projection for Dimension Reduction. arXiv:180203426 [cs, stat].
McInnes L, Healy J, Saul N, and Großberger L (2018) UMAP: Uniform Manifold Approximation and Projection. J Open Source Softw 3:861.
Mervin L H, Johansson S, Semenova E, Giblin K A, and Engkvist O (2020) Uncertainty quantification in drug design. Drug Discov Today 26:474-489.
Miljković F, Martinsson A, Obrezanova O, Williamson B, Johnson M, Sykes A, Bender A, and Greene N (2021) Machine Learning Models for Human In Vivo Pharmacokinetic Parameters with In-House Validation. Mol Pharmaceut, doi: 10.1021/acs.molpharmaceut.1c00718.
Moulavi R J G B C, Sander and J, and Moulavi D (2013) Density-Based Clustering Based on Hierarchical Density Estimates.
Murad N, Pasikanti K K, Madej B D, Minnich A, McComas J M, Crouch S, Polli J W, and Weber A D (2020a) Predicting Volume of Distribution in Humans: Performance of in silico Methods for A Large Set of Structurally Diverse Clinical Compounds. Drug Metab Dispos 49:DMD-AR-2020-000202.
Murad N, Pasikanti K K, Madej B D, Minnich A, McComas J M, Crouch S, Polli J W, and Weber A D (2020b) Predicting Volume of Distribution in Humans: Performance of in silico Methods for A Large Set of Structurally Diverse Clinical Compounds. Drug Metab Dispos 49:DMD-AR-2020-000202.
Naga D, Parrott N, Ecker G F, and Olivares-Morales A (2022) Evaluation of the Success of High-Throughput Physiologically Based Pharmacokinetic (HT-PBPK) Modeling Predictions to Inform Early Drug Discovery. Mol Pharmaceut, doi: 10.1021/acs.molpharmaceut.2c00040.
Obrezanova O, Martinsson A, Whitehead T, Mahmoud S, Bender A, Miljković F, Grabowski P, Irwin B, Oprisiu I, Conduit G, Segall M, Smith G F, Williamson B, Winiwarter S, and Greene N (2022) Prediction of In Vivo Pharmacokinetic Parameters and Time-Exposure Curves in Rats Using Machine Learning from the Chemical Structure. Mol Pharmaceut, doi: 10.1021/acs.molpharmaceut.2c00027.
Paixão P, Gouveia L F, and Morais JAG (2012) Prediction of the human oral bioavailability by using in vitro and in silico drug related parameters in a physiologically based absorption model. Int J Pharmaceut 429:84-98.
Poongavanam V, Doak B C, and Kihlberg J (2018) Opportunities and guidelines for discovery of orally absorbed drugs in beyond rule of 5 space. Curr Opin Chem Biol 44:23-29.
Poulin P, and Haddad S (2021) A New Guidance for the Prediction of Hepatic Clearance in the Early Drug Discovery and Development from the in Vitro-to-in Vivo Extrapolation Method and an Approach for Exploring Whether an Albumin-Mediated Hepatic Uptake Phenomenon Could be Present Under in Vivo Conditions. J Pharm Sci 110:2841-2858.
Prokhorenkova L, Gusev G, Vorobev A, Dorogush A V, and Gulin A (2017) CatBoost: unbiased boosting with categorical features. Arxiv.
Rogers D, and Hahn M (2010) Extended-Connectivity Fingerprints. J Chem Inf Model 50:742-754.
Sainburg T, McInnes L, and Gentner T Q (2020) Parametric UMAP: learning embeddings with deep neural networks for representation and semi-supervised learning. Arxiv.
Scalia G, Grambow C A, Pernici B, Li Y-P, and Green W H (2020) Evaluating Scalable Uncertainty Estimation Methods for Deep Learning-Based Molecular Property Prediction. J Chem Inf Model 60:2697-2717.
Schneckener S, Grimbs S, Hey J, Menz S, Osmers M, Schaper S, Hillisch A, and Goller A H (2019a) Prediction of Oral Bioavailability in Rats: Transferring Insights from in in Silico Model Outputs and Chemical Structure Parameters. J Chem Inf Model 59:4893-4905.
Schneckener S, Grimbs S, Hey J, Menz S, Osmers M, Schaper S, Hillisch A, and Goller A H (2019b) Prediction of Oral Bioavailability in Rats: Transferring Insights from in Vitro Correlations to (Deep) Machine Learning Models Using in Silico Model Outputs and Chemical Structure Parameters. J Chem Inf Model 59:4893-4905.
Schuffenhauer A, Brown N, Ertl P, Jenkins J L, Selzer P, and Hamon J (2007) Clustering and Rule-Based Classifications of Chemical Structures Evaluated in the Biological Activity Space. J Chem Inf Model 47:325-336.
Umehara K, Cantrill C, Wittwer M B, Lenarda E D, Klammers F, Ekiciler A, Parrott N, Fowler S, and Ullah M (2020) Application of the Extended Clearance Classification System (ECCS) in Drug Discovery and Development: Selection of Appropriate In Vitro Tools and Clearance Prediction. Drug Metab Dispos 48:DMD-AR-2020-000133.
Vamathevan J, Clark D, Czodrowski P, Dunham I, Ferran E, Lee G, Li B, Madabhushi A, Shah P, Spitzer M, and Zhao S (2019) Applications of machine learning in drug discovery and development. Nat Rev Drug Discov 18:463-477.
Wajima T, Fukumura K, Yano Y, and Oguma T (2002) Prediction of human clearance from animal data and molecular structural parameters using multivariate regression analysis. J Pharm Sci 91:2489-2499.
Wang Y, Liu H, Fan Y, Chen X, Yang Y, Zhu L, Zhao J, Chen Y, and Zhang Y (2019a) In Silico Prediction of Human Intravenous Pharmacokinetic Parameters with Improved Accuracy. J Chem Inf Model 59:3968-3980.
Wang Y, Liu H, Fan Y, Chen X, Yang Y, Zhu L, Zhao J, Chen Y, and Zhang Y (2019b) In Silico Prediction of Human Intravenous Pharmacokinetic Parameters with Improved Accuracy. J Chem Inf Model 59:3968-3980.
Williams C K I, and Rasmussen C E (1995) Gaussian Processes for Regression. arXiv.
Xie T, and Grossman J C (2018) Crystal Graph Convolutional Neural Networks for an Accurate and Interpretable Prediction of Material Properties. Phys Rev Lett 120:145301.
Xiong Z, Wang D, Liu X, Zhong F, Wan X, Li X, Li Z, Luo X, Chen K, Jiang H, and Zheng M (2020) Pushing the Boundaries of Molecular Representation for Drug Discovery with the Graph Attention Mechanism. J Med Chem 63:8749-8760.
Ye Z, Yang Y, Li X, Cao D, and Ouyang D (2019) An Integrated Transfer Learning and Multitask Learning Approach for Pharmacokinetic Parameter Prediction. Mol Pharmaceut 16:533-541.
Zheng X, Liang C, Wang L, Wang B, Liu Y, Feng S, Wu J Z, Gao L, Feng L, Chen L, Guo T, Shen H C, and Yun H (2018) Discovery of Benzoazepinequinoline (BAQ) Derivatives as Novel, Potent, Orally Bioavailable Respiratory Syncytial Virus Fusion Inhibitors. J Med Chem 61:10228-10241.
Zhivkova Z, and Doytchinova I (2012) Prediction of steady-state volume of distribution of acidic drugs by quantitative structure-pharmacokinetics relationships. J Pharm Sci 101:1253-1266.
Zhivkova Z D, Mandova T, and Doytchinova I (2015) Quantitative Structure-Pharmacokinetics Relationships Analysis of Basic Drugs: Volume of Distribution. J Pharm Pharm Sci 18:515-527.
Com, N., Mueller jonasmue@amazon, J., Com, A., Shirkov, Zhang, H., Larroy, P., Li, M.,
Smola, A. (2020). AutoGluon-Tabular: Robust and Accurate AutoML for Structured Data.
Danishuddin, Kumar V, Faheem M, and Lee K W (2021) A decade of machine learning-based predictive models for human pharmacokinetics: advances and challenges. Drug Discov Today, doi: 10.1016/j.drudis.2021.09.013.
Fagerholm U, Hellberg S, and Spjuth O (2021) Advances in Predictions of Oral Bioavailability of Candidate Drugs in Man with New Machine Learning Methodology. Molecules 26:2572.
Freitas A A, Limbu K, and Ghafourian T (2015) Predicting volume of distribution with decision tree-based regression methods using predicted tissue: plasma partition coefficients. J Cheminformatics 7:6.
Göller A H, Kuhnke L, Montanari F, Bonin A, Schneckener S, Laak A ter, Wichard J, Lobell M, and Hillisch A (2020) Bayer's in silico ADMET platform: a journey of machine learning over the past two decades. Drug Discov Today 25:1702-1709.
Gombar V K, and Hall S D (2013) Quantitative Structure-Activity Relationship Models of Clinical Pharmacokinetics: Clearance and Volume of Distribution. J Chem Inf Model 53:948-957.
Hosea N A, and Jones H M (2013) Predicting Pharmacokinetic Profiles Using in Silico Derived Parameters. Mol Pharmaceut 10:1207-1215.
Iwata H, Matsuo T, Mamada H, Motomura T, Matsushita M, Fujiwara T, Kazuya M, and Handa K (2021) Prediction of Total Drug Clearance in Humans Using Animal Data: Proposal of a Multimodal Learning Method Based on Deep Learning. J Pharm Sci 110:1834-1841.
Jiménez-Luna J, Grisoni F, and Schneider G (2020) Drug discovery with explainable artificial intelligence. Nat Mach Intell 2:573-584.
Kamiya Y, Handa K, Miura T, Yanagi M, Shigeta K, Hina S, Shimizu M, Kitajima M, Shono F, Funatsu K, and Yamazaki H (2021) In Silico Prediction of Input Parameters for Simplified Physiologically Based Pharmacokinetic Models for Estimating Plasma, Liver, and Kidney Exposures in Rats after Oral Doses of 246 Disparate Chemicals. Chem Res Toxicol 34:507-513.
Kosugi Y, and Hosea N (2020) Direct Comparison of Total Clearance Prediction: Computational Machine Learning Model versus Bottom-Up Approach Using In Vitro Assay. Mol Pharmaceut 17:2299-2309.
Kosugi Y, and Hosea N (2021) Prediction of Oral Pharmacokinetics Using a Combination of In Silico Descriptors and In Vitro ADME Properties. Mol Pharmaceut 18:1071-1079.
Kumar R, Sharma A, and Varadwaj P K (2011) A prediction model for oral bioavailability of drugs using physicochemical properties by support vector machine. J Nat Sci Biology Medicine 2:168-173.
Landrum, G. RDKit: Open-source cheminformatics; http://www.rdkit.org (accessed Mar. 1, 2022)
Lipinski C A (2004) Lead- and drug-like compounds: the rule-of-five revolution. Drug Discov Today Technologies 1:337-341.
Lombardo F, Bentzien J, Berellini G, and Muegge I (2021) In Silico Models of Human P K Parameters. Prediction of Volume of Distribution Using an Extensive Data Set and a Reduced Number of Parameters. J Pharm Sci 110:500-509.
Lombardo F, Berellini G, and Obach R S (2018) TREND ANALYSIS OF A DATABASE OF INTRAVENOUS PHARMACOKINETIC PARAMETERS IN HUMANS FOR 1352 DRUG COMPOUNDS. Drug Metab Dispos 46:dmd.118.082966.
Lombardo F, Desai P V, Arimoto R, Desino K E, Fischer H, Keefer C E, Petersson C, Winiwarter S, and Broccatelli F (2017) In Silico Absorption, Distribution, Metabolism, Excretion, and Pharmacokinetics (ADME-PK): Utility and Best Practices. An Industry Perspective from the International Consortium for Innovation through Quality in Pharmaceutical Development. J Med Chem 60:9097-9113.
Lundberg S M and Lee S-I (2017). A unified approach to interpreting model predictions. In Proceedings of the 31^{st} International Conference on Neural Information Processing Systems (NIPS' 17). Curran Associates Inc., Red Hook, N Y, USA, 4768-4777.
Prokhorenkova, L., Gusev, G., Vorobev, A., Dorogush, A. V., & Gulin, A. (2018). CatBoost: unbiased boosting with categorical features. Neural Information Processing Systems; Curran Associates, Inc.
Murad N, Pasikanti K K, Madej B D, Minnich A, McComas J M, Crouch S, Polli J W, and Weber A D (2020) Predicting Volume of Distribution in Humans: Performance of in silico Methods for A Large Set of Structurally Diverse Clinical Compounds. Drug Metab Dispos 49:DMD-AR-2020-000202.
Nori H, Jenkins S, Koch P, and Caruana R (2019) InterpretML: A Unified Framework for Machine Learning Interpretability. Arxiv.
Poongavanam V, Doak B C, and Kihlberg J (2018) Opportunities and guidelines for discovery of orally absorbed drugs in beyond rule of 5 space. Curr Opin Chem Biol 44:23-29.
Bharath Ramsundar, Peter Eastman, Patrick Walters, Vijay Pande, Karl Leswing, & Zhengin Wu (2019). Deep Learning for the Life Sciences. O'Reilly Media.
Sainburg T, McInnes L, and Gentner T Q (2020) Parametric UMAP: learning embeddings with deep neural networks for representation and semi-supervised learning. Arxiv.
Schneckener S, Grimbs S, Hey J, Menz S, Osmers M, Schaper S, Hillisch A, and Goller A H (2019a) Prediction of Oral Bioavailability in Rats: Transferring Insights from in in Silico Model Outputs and Chemical Structure Parameters. J Chem Inf Model 59:4893-4905.
Schneckener S, Grimbs S, Hey J, Menz S, Osmers M, Schaper S, Hillisch A, and Göller A H (2019b) Prediction of Oral Bioavailability in Rats: Transferring Insights from in Vitro Correlations to (Deep) Machine Learning Models Using in Silico Model Outputs and Chemical Structure Parameters. J Chem Inf Model 59:4893-4905.
Wajima T, Fukumura K, Yano Y, and Oguma T (2002) Prediction of human clearance from animal data and molecular structural parameters using multivariate regression analysis. J Pharm Sci 91:2489-2499.
Wang Y, Liu H, Fan Y, Chen X, Yang Y, Zhu L, Zhao J, Chen Y, and Zhang Y (2019) In Silico Prediction of Human Intravenous Pharmacokinetic Parameters with Improved Accuracy. J Chem Inf Model 59:3968-3980.
Williams, C., & Rasmussen, C. (1995). Gaussian Processes for Regression. Neural Information Processing Systems; MIT Press.
Zhivkova Z, and Doytchinova I (2012) Prediction of steady-state volume of distribution of acidic drugs by quantitative structure-pharmacokinetics relationships. J Pharm Sci 101:1253-1266.
Zhivkova Z D, Mandova T, and Doytchinova I (2015) Quantitative Structure—Pharmacokinetics Relationships Analysis of Basic Drugs: Volume of Distribution. J Pharm Pharm Sci 18:515-527.

TABLE 1
Selected studies that computationally predicted nonclinical and clinical PK properties
with indirect, direct, or hybrid approaches (sorted by publication date) .

| | | | Quantification | | |
|---|---|---|---|---|---|
| Predicted PK | Dataset type and | | of uncertainty/ | | |
| parameters | size | Methods | Explainable AI | Comments | Reference |
| F, CLp, Vss, | Rat PK studies | Graph | Yes/No | In vitro data was also | (Obrezanova et |
| AUC, Cmax, | available for | convolutional | | used as input | al., 2022) |
| t½ | 69ndicat. 3000 | neural networks, | | parameters | |
| | compounds | GP, GBM, SVM, | | | |
| | | Alchemite | | | |
| F, CLp, Vss, | 240-271 rat PK | PBPK and ML | No/No | PBPK and high- | (Naga et al., |
| AUC, Cmax | studies | models from | | throughput PBPK | 2022) |
| | | ADMET Predictor | | approaches with in | |
| | | | | vitro and/or ML data as | |
| | | | | input parameters | |
| AUCp, oral | 595 compounds | RF, GP | No/No | In vitro data was also | (Kosugi and |
| | with rat PK studies | | | used as input | Hosea, 2021a) |
| | | | | parameter | |
| F, CLp, Vss, | 1001 compounds | RF, GBM | No/No | | (Miljković et |
| AUC, Cmax, | with human PK | | | | al., 2021) |
| t½ | | | | | |
| CLp | 748 and 394 | multimodal Deep | No/Yes | Models of human CLp | (Iwata et al., |
| | compounds with | Tensor | | were trained with rat | 2021a) |
| | human and rat PK | | | CLp data | |
| | data, respectively | | | | |
| Oral F | 184 compounds | SVM, PLS | No/No | Prediction combines 9 | (Fagerholm et |
| | with human, rat, | | | ML models, 3 | al., 2021) |
| | mouse, and dog PK | | | structural alerts, and 2 | |
| | | | | PBPK models | |
| Ka, V1, CLp, | 246 compounds | PBPK | No/Partial | Authors predicted | (Kamiya et al., |
| AUC, Cmax | with rat PK | | | input parameters for | 2021) |
| | | | | PBPK models | |
| Vss | 1303 compounds | RF, SVM, XGB, | No/No | | (Lombardo et |
| | with human PK | GBM | | | al., 2021a) |
| Oral F | 1447 compounds | KNIME automated | No/No | | (Falcón-Cano et |
| | with human F | workflow | | | al., 2020) |
| CLp | 1114 compounds | RF, radial basis | No/No | Developed ML model | (Kosugi and |
| | with rat PK | function | | were more predictive | Hosea, 2020) |
| | | | | compared to IVIVE | |
| Vss | 956 compounds | RF, neural | No/No | Human Vss was | (Murad et al., |
| | with human PK | networks | | predicted directly and | 2020a) |
| | | | | indirectly, via input | |
| | | | | parameters required | |
| | | | | for mechanistic and | |
| | | | | empirical | |
| AUC, F | 1900 compounds | RF, neural | No/Partial | PK parameters were | (Schneckener |
| | with rat PK | networks | | predicted directly, | et al., 2019b) |
| | | | | indirectly (via input | |
| | | | | parameters for PBPK), | |
| | | | | and with hybrid | |
| | | | | approaches | |
| Vss, CLp, t½ | 1352 compounds | SVM, RF, GBM, | No/No | | (Wang et al., |
| | with human IV PK | XGB | | | 2019b) |
| F, Vss, t½ | 1104 compounds | neural networks | No/No | One of the first studies | (Ye et al., 2019) |
| | with human PK. | | | to apply transfer and | |
| | Pretraining with | | | multitask learning | |
| | 31 × 10⁶ compounds | | | | |
| Vss | 604 compounds | Tree-based | No/No | | (Freitas et al., |
| | with human PK | regression | | | 2015) |
| | | methods | | | |
| Vss | 216 basic drugs with | MLR | No/No | | (Zhivkova et al., |
| | human PK | | | | 2015) |
| Oral PK | 3 example | PBPK | No/Partial | GastroPlus was used | (Hosea and |
| profiles | compounds | | | with in silico or in vitro | Jones, 2013) |
| | | | | data | |
| Vss | 670 compounds | PLS | No/No | | (Amo et al., |
| | with human IV PK | | | | 2013) |
| Vss, CLp | 670 compounds | SVR, MLR | No/No | | (Gombar and |
| | with human IV PK | | | | Hall, 2013) |
| Vss | 132 acidic | GA, SR | No/No | | (Zhivkova and |
| | compounds with | | | | Doytchinova, |
| | human PK | | | | 2012) |
| F | 511 compounds | SVM | No/No | | (Kumar et al., |
| | with human PK | | | | 2011) |
| CLp | 68 compounds, rat, | MLR, ANN | No/No | | (Wajima et al., |
| | dog, human | | | | 2002) |

TABLE 2
Performance of AutoGluon, ChemProp, and GP Single-Task models for predictions of
mouse CLp (mL/min/kg), Vss (L/kg), and oral F (%). Performance metrics are generated
with a random split and 5-fold cross-validation process. Best performance values
with respect to each metric are highlighted. (M = mean; SD = standard deviation)

| | | AAFE | | RMSLE | | <3-fold | | AFE | | R2(log10) | | CCC | |
|---|---|---|---|---|---|---|---|---|---|---|---|---|---|
| Model | Property | M | SD | M | SD | M | SD | M | SD | M | SD | M | SD |
| AutoGluon | CLp | 2.09 | 0.04 | 0.43 | 0.01 | 78% | 2% | 1.00 | 0.05 | 0.48 | 0.02 | 0.65 | 0.01 |
| ChemProp | CLP | 2.24 | 0.06 | 0.46 | 0.02 | 73% | 2% | 1.03 | 0.05 | 0.40 | 0.04 | 0.6 | 0.03 |
| GP | CLp | 2.08 | 0.02 | 0.43 | 0.01 | 78% | 1% | 1.00 | 0.04 | 0.48 | 0.03 | 0.66 | 0.01 |
| AutoGluon | F | 2.28 | 0.07 | 0.52 | 0.01 | 77% | 3% | 1.42 | 0.03 | 0.15 | 0.06 | 0.31 | 0.04 |
| ChemProp | F | 2.39 | 0.08 | 0.54 | 0.02 | 74% | 1% | 1.38 | 0.20 | 0.11 | 0.04 | 0.28 | 0.04 |
| GP | F | 2.23 | 0.08 | 0.50 | 0.02 | 77% | 3% | 1.39 | 0.10 | 0.20 | 0.03 | 0.36 | 0.01 |
| AutoGluon | Vss | 1.98 | 0.02 | 0.40 | 0.01 | 81% | 1% | 0.97 | 0.03 | 0.47 | 0.02 | 0.65 | 0.02 |
| ChemProp | Vss | 2.08 | 0.07 | 0.42 | 0.03 | 79% | 2% | 1.02 | 0.08 | 0.40 | 0.06 | 0.6 | 0.04 |
| GP | Vss | 1.98 | 0.03 | 0.40 | 0.02 | 81% | 1% | 1.00 | 0.03 | 0.47 | 0.04 | 0.65 | 0.02 |

TABLE 3
A number of unique chemical structures and corresponding aggregated
mouse and rat PK measurements available in May 2022.

| PK Property | Unit | Mouse | Rat | Species overlap |
|---|---|---|---|---|
| AUCiv | h*ng/mL/mg/kg | 2730 | 2678 | 8% |
| AUCpo | h*ng/mL/mg/kg | 3820 | 2616 | 16% |
| CLp | mL/min/kg | 3515 | 3895 | 8% |
| F | % | 2091 | 2949 | 24% |
| Vss | L/kg | 3448 | 3871 | 8% |

| | | | |
|---|---|---|---|
| Number of unique compounds and PK | 5952 | 4478 | 9% |
| results per species | | | |

| | |
|---|---|
| Overall number of unique compounds | 9685 |
| and PK results | |

TABLE 4
Descriptive statistics of molecular properties in the preprocessed in vivo dataset.

| Property | MW | TPSA | H Bond | H Bond | | | Ionization |
|---|---|---|---|---|---|---|---|
| [Unit] | [Da] | [Å²] | Acceptors | Donors | logD | AlogP | Category |
| All compounds | | | | | | | |
| Median | 464 | 100 | 7 | 2 | 2.61 | 3.80 | Neutral 49% |
| Average | 472 | 101 | 7.5 | 1.7 | 2.50 | 3.90 | Acidic 16% |
| Min | 123 | 0 | 0 | 0 | −2.46 | −2.48 | Basic 31% |
| Max | 988 | 395 | 23 | 11 | 6.76 | 10.7 | Zwitterionic 4% |

Compounds tested in mouse PK studies

| | | | | | | | |
|---|---|---|---|---|---|---|---|
| Median | 472 | 104 | 8 | 2 | 2.68 | 3.91 | Neutral 47% |
| Average | 481 | 105 | 7.7 | 1.7 | 2.56 | 3.94 | Acidic 17% |
| Min | 123 | 12 | 1 | 0 | −2.46 | −2.48 | Basic 32% |
| Max | 988 | 395 | 23 | 11 | 6.00 | 10.69 | Zwitterionic 4% |

Compounds tested in rat PK studies

| | | | | | | | |
|---|---|---|---|---|---|---|---|
| Median | 448 | 94 | 7 | 2 | 2.57 | 3.70 | Neutral 52% |
| Average | 460 | 95 | 7.2 | 1.6 | 2.47 | 3.80 | Acidic 14% |
| Min | 123 | 0 | 0 | 0 | −2.46 | −2.48 | Basic 29% |
| Max | 958 | 254 | 18 | 7 | 6.76 | 10.69 | Zwitterionic 5% |

TABLE 5
Descriptive statistics of observed pharmacokinetic properties
in mouse and rat pharmacokinetic studies.

| PK | | Mouse | | | | Rat | | | |
|---|---|---|---|---|---|---|---|---|---|
| property | Unit | Median | Average | Min | Max | Median | Average | Min | Max |
| CLp | mL/min/kg | 25.4 | 48.6 | 0.3 | 424.9 | 27.3 | 41.7 | 0.4 | 294.3 |
| Vss | L/kg | 1.86 | 4.15 | 0.1 | 49.61 | 2.25 | 5.38 | 0.1 | 63.35 |
| AUCiv | h*ng/ml/mg/ | 689 | 3265 | 34 | 78625 | 631 | 2315 | 49 | 38146 |
| | kg | | | | | | | | |
| AUCpo | h*ng/ml/mg/ | 273 | 1173 | 1 | 17900 | 196 | 970 | 1 | 15573 |
| | kg | | | | | | | | |
| F | % | 36.0 | 41.1 | 0.0 | 100.0 | 29.1 | 36.8 | 0.0 | 100.0 |

TABLE 6
Estimated experimental AAFE errors
or replicate in vivo measurements.

| PK property | Unit | AAFEₑₓₚ Mouse | AAFEₑₓₚ Rat |
|---|---|---|---|
| CLp | mL/min/kg | 1.19 | 1.27 |
| Vss | L/kg | 1.23 | 1.31 |
| AUCiv | h*ng/mL/mg/kg | 1.22 | 1.37 |
| AUCpo | h*ng/mL/mg/kg | 1.50 | 1.77 |
| F | % | 1.19 | 1.41 |
| Average | | 1.27 | 1.43 |

TABLE 7
Performance of all Single-Task ML models evaluated on a test set
using a random data split and 5-fold cross-validation process.

| | | | AAFE | | RMSLE | | AFE | | R2(log10) | | CCC | |
|---|---|---|---|---|---|---|---|---|---|---|---|---|
| Model | Species | Property | M | SD | M | SD | M | SD | M | SD | M | SD |
| AutoGluon | Mouse | AUCiv | 2.11 | 0.08 | 0.44 | 0.02 | 0.99 | 0.05 | 0.50 | 0.03 | 0.67 | 0.02 |
| AutoGluon | Mouse | AUCpo | 3.15 | 0.09 | 0.67 | 0.02 | 1.02 | 0.08 | 0.47 | 0.03 | 0.64 | 0.02 |
| AutoGluon | Mouse | CLp | 2.09 | 0.04 | 0.43 | 0.01 | 1.00 | 0.05 | 0.48 | 0.02 | 0.65 | 0.01 |
| AutoGluon | Mouse | F | 2.28 | 0.07 | 0.52 | 0.01 | 1.42 | 0.03 | 0.15 | 0.06 | 0.31 | 0.04 |
| AutoGluon | Mouse | Vss | 1.98 | 0.02 | 0.40 | 0.01 | 0.97 | 0.03 | 0.47 | 0.02 | 0.65 | 0.02 |
| AutoGluon | Rat | AUCiv | 1.97 | 0.05 | 0.39 | 0.02 | 0.98 | 0.04 | 0.52 | 0.03 | 0.68 | 0.01 |
| AutoGluon | Rat | AUCpo | 3.45 | 0.15 | 0.70 | 0.03 | 1.05 | 0.06 | 0.43 | 0.02 | 0.61 | 0.02 |
| AutoGluon | Rat | CLp | 2.00 | 0.02 | 0.40 | 0.01 | 0.99 | 0.04 | 0.51 | 0.03 | 0.67 | 0.02 |
| AutoGluon | Rat | F | 2.59 | 0.13 | 0.63 | 0.05 | 1.78 | 0.22 | 0.16 | 0.05 | 0.30 | 0.05 |
| AutoGluon | Rat | Vss | 1.93 | 0.04 | 0.38 | 0.01 | 0.97 | 0.03 | 0.53 | 0.02 | 0.68 | 0.01 |
| CatBoost | Mouse | AUCiv | 2.19 | 0.08 | 0.46 | 0.02 | 1.00 | 0.06 | 0.47 | 0.02 | 0.64 | 0.02 |
| CatBoost | Mouse | AUCpo | 3.33 | 0.10 | 0.69 | 0.02 | 1.04 | 0.08 | 0.43 | 0.03 | 0.58 | 0.02 |
| CatBoost | Mouse | CLp | 2.25 | 0.05 | 0.47 | 0.01 | 1.01 | 0.06 | 0.39 | 0.02 | 0.53 | 0.01 |
| CatBoost | Mouse | F | 2.24 | 0.09 | 0.51 | 0.03 | 1.43 | 0.10 | 0.18 | 0.05 | 0.33 | 0.02 |
| CatBoost | Mouse | Vss | 2.03 | 0.02 | 0.41 | 0.02 | 1.00 | 0.02 | 0.44 | 0.03 | 0.62 | 0.02 |
| CatBoost | Rat | AUCiv | 2.09 | 0.05 | 0.42 | 0.01 | 1.00 | 0.04 | 0.45 | 0.02 | 0.61 | 0.01 |
| CatBoost | Rat | AUCpo | 3.61 | 0.14 | 0.73 | 0.03 | 1.03 | 0.08 | 0.39 | 0.02 | 0.59 | 0.02 |
| CatBoost | Rat | CLp | 2.10 | 0.05 | 0.43 | 0.01 | 1.00 | 0.04 | 0.44 | 0.03 | 0.61 | 0.02 |
| CatBoost | Rat | F | 2.72 | 0.11 | 0.64 | 0.04 | 1.48 | 0.11 | 0.08 | 0.08 | 0.33 | 0.01 |
| CatBoost | Rat | Vss | 2.00 | 0.04 | 0.40 | 0.01 | 0.99 | 0.04 | 0.49 | 0.02 | 0.64 | 0.01 |
| ChemProp | Mouse | AUCiv | 2.29 | 0.13 | 0.48 | 0.04 | 1.00 | 0.04 | 0.42 | 0.05 | 0.61 | 0.04 |
| ChemProp | Mouse | AUCpo | 3.49 | 0.12 | 0.71 | 0.02 | 1.11 | 0.10 | 0.38 | 0.04 | 0.58 | 0.02 |
| ChemProp | Mouse | CLp | 2.24 | 0.06 | 0.46 | 0.02 | 1.03 | 0.05 | 0.40 | 0.04 | 0.60 | 0.03 |
| ChemProp | Mouse | F | 2.39 | 0.08 | 0.54 | 0.02 | 1.38 | 0.20 | 0.11 | 0.04 | 0.28 | 0.04 |
| ChemProp | Mouse | Vss | 2.08 | 0.07 | 0.42 | 0.03 | 1.02 | 0.08 | 0.40 | 0.06 | 0.60 | 0.04 |
| ChemProp | Rat | AUCiv | 2.07 | 0.04 | 0.41 | 0.01 | 1.02 | 0.10 | 0.47 | 0.02 | 0.65 | 0.02 |
| ChemProp | Rat | AUCpo | 3.93 | 0.21 | 0.77 | 0.03 | 0.97 | 0.09 | 0.33 | 0.03 | 0.52 | 0.03 |
| ChemProp | Rat | CLp | 2.12 | 0.06 | 0.43 | 0.02 | 1.03 | 0.08 | 0.42 | 0.03 | 0.63 | 0.03 |
| ChemProp | Rat | F | 2.71 | 0.16 | 0.64 | 0.05 | 1.57 | 0.26 | 0.13 | 0.11 | 0.33 | 0.03 |
| ChemProp | Rat | Vss | 2.02 | 0.03 | 0.40 | 0.01 | 1.01 | 0.11 | 0.48 | 0.02 | 0.66 | 0.02 |
| DeepChem | Mouse | AUCiv | 2.35 | 0.08 | 0.49 | 0.03 | 1.00 | 0.17 | 0.38 | 0.05 | 0.58 | 0.03 |
| AttentiveFP | | | | | | | | | | | | |
| DeepChem | Mouse | AUCpo | 3.45 | 0.16 | 0.71 | 0.03 | 1.03 | 0.18 | 0.40 | 0.05 | 0.62 | 0.02 |
| AttentiveFP | | | | | | | | | | | | |
| DeepChem | Mouse | CLp | 2.26 | 0.07 | 0.47 | 0.02 | 0.98 | 0.10 | 0.38 | 0.06 | 0.59 | 0.03 |
| AttentiveFP | | | | | | | | | | | | |
| DeepChem | Mouse | F | 2.38 | 0.08 | 0.53 | 0.03 | 1.32 | 0.17 | 0.10 | 0.06 | 0.36 | 0.01 |
| AttentiveFP | | | | | | | | | | | | |
| DeepChem | Mouse | Vss | 2.09 | 0.06 | 0.43 | 0.02 | 1.00 | 0.13 | 0.39 | 0.05 | 0.60 | 0.02 |
| AttentiveFP | | | | | | | | | | | | |
| DeepChem | Rat | AUCiv | 2.15 | 0.11 | 0.44 | 0.03 | 1.02 | 0.10 | 0.42 | 0.07 | 0.63 | 0.04 |
| AttentiveFP | | | | | | | | | | | | |
| DeepChem | Rat | AUCpo | 3.95 | 0.21 | 0.77 | 0.03 | 1.15 | 0.24 | 0.32 | 0.04 | 0.54 | 0.04 |
| AttentiveFP | | | | | | | | | | | | |
| DeepChem | Rat | CLp | 2.16 | 0.07 | 0.44 | 0.02 | 0.98 | 0.15 | 0.40 | 0.02 | 0.62 | 0.02 |
| AttentiveFP | | | | | | | | | | | | |
| DeepChem | Rat | F | 2.62 | 0.11 | 0.61 | 0.05 | 1.42 | 0.23 | 0.10 | 0.06 | 0.33 | 0.05 |
| AttentiveFP | | | | | | | | | | | | |
| DeepChem | Rat | Vss | 2.01 | 0.04 | 0.40 | 0.01 | 0.99 | 0.09 | 0.49 | 0.03 | 0.68 | 0.03 |
| AttentiveFP | | | | | | | | | | | | |
| DeepChem | Mouse | AUCiv | 2.28 | 0.08 | 0.47 | 0.02 | 1.00 | 0.12 | 0.43 | 0.02 | 0.66 | 0.02 |
| GCNN | | | | | | | | | | | | |
| DeepChem | Mouse | AUCpo | 3.60 | 0.12 | 0.73 | 0.03 | 1.09 | 0.46 | 0.37 | 0.04 | 0.62 | 0.02 |
| GCNN | | | | | | | | | | | | |
| DeepChem | Mouse | CLp | 2.26 | 0.05 | 0.47 | 0.02 | 1.00 | 0.09 | 0.38 | 0.02 | 0.62 | 0.01 |
| GCNN | | | | | | | | | | | | |
| DeepChem | Mouse | F | 2.57 | 0.12 | 0.58 | 0.03 | 1.18 | 0.23 | −0.06 | 0.11 | 0.33 | 0.03 |
| GCNN | | | | | | | | | | | | |
| DeepChem | Mouse | Vss | 2.09 | 0.05 | 0.43 | 0.02 | 0.97 | 0.08 | 0.39 | 0.05 | 0.63 | 0.03 |
| GCNN | | | | | | | | | | | | |
| DeepChem | Rat | AUCiv | 2.15 | 0.05 | 0.44 | 0.02 | 1.09 | 0.10 | 0.41 | 0.03 | 0.65 | 0.02 |
| GCNN | | | | | | | | | | | | |
| DeepChem | Rat | AUCpo | 4.01 | 0.31 | 0.79 | 0.05 | 1.00 | 0.13 | 0.29 | 0.07 | 0.55 | 0.06 |
| GCNN | | | | | | | | | | | | |
| DeepChem | Rat | CLp | 2.19 | 0.02 | 0.45 | 0.01 | 0.92 | 0.14 | 0.39 | 0.04 | 0.63 | 0.02 |
| GCNN | | | | | | | | | | | | |
| DeepChem | Rat | F | 3.13 | 0.12 | 0.72 | 0.05 | 1.40 | 0.34 | −0.01 | 0.11 | 0.37 | 0.03 |
| GCNN | | | | | | | | | | | | |
| DeepChem | Rat | Vss | 2.07 | 0.02 | 0.42 | 0.01 | 0.99 | 0.05 | 0.44 | 0.03 | 0.67 | 0.01 |
| GCNN | | | | | | | | | | | | |
| DeepChem RF | Mouse | AUCiv | 2.13 | 0.09 | 0.45 | 0.02 | 1.01 | 0.05 | 0.49 | 0.02 | 0.64 | 0.02 |
| DeepChem RF | Mouse | AUCpo | 3.14 | 0.12 | 0.67 | 0.02 | 1.08 | 0.07 | 0.46 | 0.03 | 0.61 | 0.02 |
| DeepChem RF | Mouse | CLp | 2.11 | 0.04 | 0.44 | 0.01 | 1.00 | 0.04 | 0.46 | 0.01 | 0.61 | 0.01 |
| DeepChem RF | Mouse | F | 2.24 | 0.11 | 0.51 | 0.03 | 1.43 | 0.10 | 0.17 | 0.07 | 0.34 | 0.03 |
| DeepChem RF | Mouse | Vss | 2.00 | 0.03 | 0.40 | 0.01 | 1.00 | 0.03 | 0.46 | 0.03 | 0.62 | 0.02 |
| DeepChem RF | Rat | AUCiv | 2.00 | 0.05 | 0.40 | 0.02 | 1.01 | 0.04 | 0.50 | 0.02 | 0.65 | 0.01 |
| DeepChem RF | Rat | AUCpo | 3.53 | 0.17 | 0.71 | 0.03 | 1.07 | 0.08 | 0.41 | 0.03 | 0.56 | 0.03 |
| DeepChem RF | Rat | CLp | 2.05 | 0.03 | 0.41 | 0.01 | 0.98 | 0.04 | 0.48 | 0.04 | 0.62 | 0.03 |
| DeepChem RF | Rat | F | 2.56 | 0.12 | 0.63 | 0.05 | 1.71 | 0.12 | 0.14 | 0.05 | 0.32 | 0.03 |
| DeepChem RF | Rat | Vss | 1.96 | 0.03 | 0.39 | 0.01 | 0.99 | 0.05 | 0.51 | 0.01 | 0.66 | 0.01 |
| EBM | Mouse | AUCiv | 2.33 | 0.08 | 0.49 | 0.02 | 1.01 | 0.05 | 0.39 | 0.02 | 0.57 | 0.01 |
| EBM | Mouse | AUCpo | 3.59 | 0.14 | 0.72 | 0.02 | 1.01 | 0.07 | 0.37 | 0.05 | 0.56 | 0.03 |
| EBM | Mouse | CLp | 2.29 | 0.03 | 0.47 | 0.01 | 1.00 | 0.04 | 0.37 | 0.03 | 0.55 | 0.02 |
| EBM | Mouse | F | 2.30 | 0.09 | 0.53 | 0.03 | 1.41 | 0.10 | 0.14 | 0.05 | 0.29 | 0.02 |
| EBM | Mouse | Vss | 2.12 | 0.03 | 0.43 | 0.01 | 1.00 | 0.02 | 0.38 | 0.05 | 0.56 | 0.03 |
| EBM | Rat | AUCiv | 2.15 | 0.06 | 0.44 | 0.02 | 1.01 | 0.04 | 0.40 | 0.02 | 0.58 | 0.01 |
| EBM | Rat | AUCpo | 3.85 | 0.11 | 0.76 | 0.02 | 1.00 | 0.05 | 0.34 | 0.03 | 0.52 | 0.02 |
| EBM | Rat | CLp | 2.20 | 0.05 | 0.45 | 0.02 | 1.00 | 0.04 | 0.38 | 0.02 | 0.56 | 0.02 |
| EBM | Rat | F | 2.66 | 0.14 | 0.64 | 0.05 | 1.71 | 0.17 | 0.13 | 0.05 | 0.28 | 0.01 |
| EBM | Rat | Vss | 2.12 | 0.03 | 0.43 | 0.01 | 1.00 | 0.03 | 0.41 | 0.02 | 0.59 | 0.02 |
| GP | Mouse | AUCiv | 2.11 | 0.07 | 0.44 | 0.02 | 1.01 | 0.06 | 0.50 | 0.03 | 0.67 | 0.02 |
| GP | Mouse | AUCpo | 3.11 | 0.09 | 0.66 | 0.02 | 1.03 | 0.06 | 0.48 | 0.04 | 0.66 | 0.02 |
| GP | Mouse | CLp | 2.08 | 0.02 | 0.43 | 0.01 | 1.00 | 0.04 | 0.48 | 0.03 | 0.66 | 0.01 |
| GP | Mouse | F | 2.23 | 0.08 | 0.50 | 0.02 | 1.39 | 0.10 | 0.20 | 0.03 | 0.36 | 0.01 |
| GP | Mouse | Vss | 1.98 | 0.03 | 0.40 | 0.02 | 1.00 | 0.03 | 0.47 | 0.04 | 0.65 | 0.02 |
| GP | Rat | AUCiv | 2.00 | 0.07 | 0.40 | 0.02 | 1.02 | 0.05 | 0.51 | 0.04 | 0.68 | 0.02 |
| GP | Rat | AUCpo | 3.47 | 0.19 | 0.70 | 0.04 | 1.03 | 0.07 | 0.43 | 0.03 | 0.61 | 0.03 |
| GP | Rat | CLp | 2.01 | 0.04 | 0.40 | 0.01 | 0.99 | 0.04 | 0.50 | 0.03 | 0.67 | 0.02 |
| GP | Rat | F | 2.55 | 0.10 | 0.61 | 0.05 | 1.64 | 0.13 | 0.19 | 0.05 | 0.35 | 0.01 |
| GP | Rat | Vss | 1.96 | 0.04 | 0.39 | 0.01 | 0.99 | 0.04 | 0.51 | 0.03 | 0.68 | 0.01 |
| NGBoost | Mouse | AUCiv | 2.44 | 0.12 | 0.51 | 0.03 | 1.01 | 0.06 | 0.34 | 0.02 | 0.45 | 0.02 |
| NGBoost | Mouse | AUCpo | 3.94 | 0.13 | 0.76 | 0.02 | 1.00 | 0.08 | 0.30 | 0.03 | 0.40 | 0.02 |
| NGBoost | Mouse | CLp | 2.45 | 0.08 | 0.50 | 0.02 | 1.00 | 0.06 | 0.29 | 0.01 | 0.39 | 0.01 |
| NGBoost | Mouse | F | 2.33 | 0.07 | 0.54 | 0.02 | 1.49 | 0.11 | 0.12 | 0.03 | 0.20 | 0.01 |
| NGBoost | Mouse | Vss | 2.27 | 0.05 | 0.46 | 0.01 | 1.00 | 0.03 | 0.30 | 0.01 | 0.39 | 0.01 |
| NGBoost | Rat | AUCiv | 2.24 | 0.07 | 0.46 | 0.02 | 1.01 | 0.04 | 0.37 | 0.01 | 0.48 | 0.02 |
| NGBoost | Rat | AUCpo | 4.13 | 0.18 | 0.79 | 0.02 | 1.01 | 0.08 | 0.29 | 0.02 | 0.39 | 0.01 |
| NGBoost | Rat | CLp | 2.33 | 0.08 | 0.48 | 0.02 | 1.00 | 0.04 | 0.31 | 0.02 | 0.41 | 0.02 |
| NGBoost | Rat | F | 2.73 | 0.14 | 0.66 | 0.05 | 1.97 | 0.16 | 0.12 | 0.02 | 0.21 | 0.01 |
| NGBoost | Rat | Vss | 2.28 | 0.08 | 0.46 | 0.02 | 1.00 | 0.04 | 0.32 | 0.01 | 0.41 | 0.02 |
| SVR | Mouse | AUCiv | 2.14 | 0.05 | 0.45 | 0.02 | 1.00 | 0.05 | 0.48 | 0.03 | 0.64 | 0.02 |
| SVR | Mouse | AUCpo | 3.24 | 0.13 | 0.69 | 0.02 | 1.12 | 0.08 | 0.43 | 0.03 | 0.59 | 0.02 |
| SVR | Mouse | CLp | 2.12 | 0.02 | 0.44 | 0.01 | 1.01 | 0.05 | 0.47 | 0.03 | 0.62 | 0.02 |
| SVR | Mouse | F | 2.22 | 0.08 | 0.50 | 0.02 | 1.32 | 0.09 | 0.17 | 0.02 | 0.25 | 0.02 |
| SVR | Mouse | Vss | 2.01 | 0.02 | 0.40 | 0.01 | 0.99 | 0.03 | 0.45 | 0.04 | 0.61 | 0.02 |
| SVR | Rat | AUCiv | 2.02 | 0.06 | 0.41 | 0.02 | 1.02 | 0.05 | 0.49 | 0.02 | 0.64 | 0.01 |
| SVR | Rat | AUCpo | 3.56 | 0.18 | 0.72 | 0.03 | 1.09 | 0.07 | 0.41 | 0.03 | 0.56 | 0.03 |
| SVR | Rat | CLp | 2.04 | 0.04 | 0.41 | 0.01 | 0.99 | 0.04 | 0.48 | 0.02 | 0.64 | 0.01 |
| SVR | Rat | F | 2.47 | 0.09 | 0.59 | 0.05 | 1.34 | 0.04 | 0.16 | 0.03 | 0.33 | 0.02 |
| SVR | Rat | Vss | 1.98 | 0.04 | 0.40 | 0.01 | 0.99 | 0.03 | 0.50 | 0.02 | 0.64 | 0.01 |

TABLE 8
Performance of all Multi-Task ML models evaluated on a test set
using a random data split and 5-fold cross-validation process

| | | | AAFE | | RMSLE | | AFE | | R2(log10) | | CCC | |
|---|---|---|---|---|---|---|---|---|---|---|---|---|
| Model | Species | Property | M | SD | M | SD | M | SD | M | SD | M | SD |
| AutoGluon | Mouse | AUCiv | 2.10 | 0.07 | 0.44 | 0.02 | 0.99 | 0.06 | 0.50 | 0.03 | 0.67 | 0.02 |
| AutoGluon | Mouse | AUCpo | 3.28 | 0.18 | 0.69 | 0.03 | 1.26 | 0.11 | 0.42 | 0.04 | 0.6 | 0.03 |
| AutoGluon | Mouse | CLp | 2.19 | 0.06 | 0.46 | 0.01 | 0.95 | 0.05 | 0.41 | 0.02 | 0.6 | 0.02 |
| AutoGluon | Mouse | F | 2.32 | 0.14 | 0.52 | 0.02 | 1.25 | 0.03 | 0.11 | 0.09 | 0.34 | 0.03 |
| AutoGluon | Mouse | Vss | 2.03 | 0.02 | 0.41 | 0.01 | 1.03 | 0.05 | 0.44 | 0.03 | 0.61 | 0.02 |
| AutoGluon | Rat | AUCiv | 1.98 | 0.05 | 0.39 | 0.02 | 1.02 | 0.06 | 0.53 | 0.02 | 0.68 | 0.01 |
| AutoGluon | Rat | AUCpo | 3.61 | 0.15 | 0.72 | 0.03 | 1.06 | 0.14 | 0.40 | 0.02 | 0.58 | 0.03 |
| AutoGluon | Rat | CLp | 2.10 | 0.05 | 0.43 | 0.02 | 1.05 | 0.07 | 0.44 | 0.02 | 0.6 | 0.02 |
| AutoGluon | Rat | F | 2.63 | 0.12 | 0.61 | 0.06 | 1.15 | 0.20 | 0.09 | 0.04 | 0.26 | 0.03 |
| AutoGluon | Rat | Vss | 1.97 | 0.06 | 0.39 | 0.02 | 0.97 | 0.04 | 0.50 | 0.01 | 0.66 | 0.01 |
| ChemProp | Mouse | AUCiv | 2.24 | 0.10 | 0.47 | 0.03 | 0.99 | 0.10 | 0.44 | 0.05 | 0.64 | 0.02 |
| ChemProp | Mouse | AUCpo | 3.56 | 0.11 | 0.72 | 0.02 | 1.04 | 0.07 | 0.38 | 0.03 | 0.58 | 0.02 |
| ChemProp | Mouse | CLp | 2.22 | 0.08 | 0.46 | 0.03 | 1.03 | 0.07 | 0.41 | 0.05 | 0.61 | 0.02 |
| ChemProp | Mouse | F | 2.31 | 0.10 | 0.52 | 0.02 | 1.37 | 0.07 | 0.15 | 0.04 | 0.32 | 0.03 |
| ChemProp | Mouse | Vss | 2.07 | 0.06 | 0.42 | 0.03 | 0.99 | 0.05 | 0.41 | 0.06 | 0.61 | 0.03 |
| ChemProp | Rat | AUCiv | 2.01 | 0.07 | 0.40 | 0.02 | 0.98 | 0.09 | 0.51 | 0.03 | 0.69 | 0.02 |
| ChemProp | Rat | AUCpo | 3.83 | 0.24 | 0.74 | 0.04 | 1.02 | 0.20 | 0.37 | 0.04 | 0.56 | 0.04 |
| ChemProp | Rat | CLp | 2.09 | 0.04 | 0.42 | 0.01 | 1.04 | 0.12 | 0.45 | 0.02 | 0.65 | 0.02 |
| ChemProp | Rat | F | 2.69 | 0.10 | 0.64 | 0.04 | 1.64 | 0.15 | 0.16 | 0.07 | 0.34 | 0.02 |
| ChemProp | Rat | Vss | 2.02 | 0.03 | 0.40 | 0.01 | 1.00 | 0.07 | 0.48 | 0.02 | 0.67 | 0.02 |
| DeepChem | Mouse | AUCiv | 2.43 | 0.18 | 0.51 | 0.05 | 1.07 | 0.29 | 0.34 | 0.10 | 0.62 | 0.05 |
| AttentiveFP | | | | | | | | | | | | |
| DeepChem | Mouse | AUCpo | 3.64 | 0.22 | 0.73 | 0.03 | 0.90 | 0.13 | 0.35 | 0.06 | 0.59 | 0.04 |
| AttentiveFP | | | | | | | | | | | | |
| DeepChem | Mouse | CLp | 2.34 | 0.06 | 0.49 | 0.02 | 0.96 | 0.07 | 0.33 | 0.03 | 0.59 | 0.02 |
| AttentiveFP | | | | | | | | | | | | |
| DeepChem | Mouse | F | 2.43 | 0.10 | 0.54 | 0.02 | 1.27 | 0.15 | 0.04 | 0.07 | 0.39 | 0.02 |
| AttentiveFP | | | | | | | | | | | | |
| DeepChem | Mouse | Vss | 2.19 | 0.08 | 0.44 | 0.03 | 1.03 | 0.03 | 0.34 | 0.07 | 0.58 | 0.04 |
| AttentiveFP | | | | | | | | | | | | |
| DeepChem | Rat | AUCiv | 2.17 | 0.07 | 0.44 | 0.02 | 1.02 | 0.16 | 0.41 | 0.05 | 0.65 | 0.03 |
| AttentiveFP | | | | | | | | | | | | |
| DeepChem | Rat | AUCpo | 4.03 | 0.25 | 0.78 | 0.04 | 1.14 | 0.22 | 0.31 | 0.05 | 0.56 | 0.04 |
| AttentiveFP | | | | | | | | | | | | |
| DeepChem | Rat | CLp | 2.23 | 0.07 | 0.45 | 0.02 | 1.00 | 0.07 | 0.37 | 0.03 | 0.6 | 0.02 |
| AttentiveFP | | | | | | | | | | | | |
| DeepChem | Rat | F | 2.69 | 0.09 | 0.62 | 0.03 | 1.38 | 0.18 | 0.03 | 0.10 | 0.39 | 0.04 |
| AttentiveFP | | | | | | | | | | | | |
| DeepChem | Rat | Vss | 2.14 | 0.02 | 0.43 | 0.02 | 1.01 | 0.13 | 0.40 | 0.02 | 0.62 | 0.02 |
| AttentiveFP | | | | | | | | | | | | |

TABLE 9
Performance of pretrained ChemProp models evacuated on a test set
using random data split and 5-fold cross-validation process.

| | | | | AAFE | | RMSLE | | AFE | | R2(log10) | | CCC | |
|---|---|---|---|---|---|---|---|---|---|---|---|---|---|
| Model | Species | Property | Pretraining | M | SD | M | SD | M | SD | M | SD | M | SD |
| ChemProp | Mouse | CLp | None | 2.24 | 0.06 | 0.46 | 0.02 | 1.03 | 0.05 | 0.40 | 0.04 | 0.60 | 0.03 |
| ChemProp | Mouse | CLp | Hepatocytes | 2.16 | 0.08 | 0.45 | 0.02 | 1.01 | 0.13 | 0.43 | 0.06 | 0.67 | 0.03 |
| ChemProp | Mouse | CLp | Microsomes | 2.18 | 0.03 | 0.45 | 0.01 | 1.04 | 0.08 | 0.42 | 0.03 | 0.67 | 0.02 |
| ChemProp | Mouse | CLp | Extrapolated | 2.16 | 0.03 | 0.45 | 0.01 | 1.01 | 0.13 | 0.44 | 0.04 | 0.67 | 0.02 |
| | | | CLp | | | | | | | | | | |
| ChemProp | Rat | CLp | None | 2.12 | 0.06 | 0.43 | 0.02 | 1.03 | 0.08 | 0.42 | 0.03 | 0.63 | 0.03 |
| ChemProp | Rat | CLp | Hepatocytes | 2.08 | 0.03 | 0.42 | 0.01 | 0.99 | 0.04 | 0.46 | 0.02 | 0.66 | 0.02 |
| ChemProp | Rat | CLp | Microsomes | 2.05 | 0.05 | 0.42 | 0.02 | 1.06 | 0.06 | 0.46 | 0.02 | 0.68 | 0.02 |
| ChemProp | Mouse | CLp | Microsomes | 2.11 | 0.03 | 0.44 | 0.01 | 0.98 | 0.06 | 0.47 | 0.03 | 0.68 | 0.02 |
| Large | | | | | | | | | | | | | |
| ChemProp | Mouse | CLp | Extrapolated | 2.13 | 0.05 | 0.44 | 0.02 | 1.01 | 0.10 | 0.45 | 0.05 | 0.68 | 0.02 |
| Large | | | CLp | | | | | | | | | | |
| ChemProp | Rat | CLp | Microsomes | 2.03 | 0.06 | 0.41 | 0.02 | 1.00 | 0.05 | 0.48 | 0.03 | 0.68 | 0.02 |
| Large | | | | | | | | | | | | | |
| ChemProp | Mouse | F | None | 2.39 | 0.08 | 0.54 | 0.02 | 1.38 | 0.20 | 0.11 | 0.04 | 0.28 | 0.04 |
| ChemProp | Mouse | F | Solubility | 2.39 | 0.07 | 0.54 | 0.03 | 1.28 | 0.29 | 0.09 | 0.03 | 0.30 | 0.04 |
| ChemProp | Mouse | F | Permeability | 2.33 | 0.11 | 0.52 | 0.02 | 1.25 | 0.10 | 0.15 | 0.04 | 0.38 | 0.01 |
| ChemProp | Rat | F | Solubility | 2.88 | 0.12 | 0.67 | 0.04 | 1.53 | 0.42 | 0.12 | 0.04 | 0.33 | 0.03 |
| ChemProp | Rat | F | Permeability | 2.81 | 0.16 | 0.66 | 0.05 | 1.43 | 0.25 | 0.11 | 0.11 | 0.36 | 0.04 |
| ChemProp | Rat | F | None | 2.71 | 0.16 | 0.64 | 0.05 | 1.57 | 0.26 | 0.13 | 0.11 | 0.33 | 0.03 |

TABLE 10
Influence of data splitting approach on performance of AutoGluon,
GP, and ChemProp models (evaluated on a test set) .

| | | | | AAFE | | RMSLE | | AFE | | R2(log10) | | CCC | |
|---|---|---|---|---|---|---|---|---|---|---|---|---|---|
| Model | Species | Property | Split | M | SD | M | SD | M | SD | M | SD | M | SD |
| AutoGluon | Mouse | AUCiv | Random | 2.11 | 0.08 | 0.44 | 0.02 | 0.99 | 0.05 | 0.50 | 0.03 | 0.67 | 0.02 |
| AutoGluon | Mouse | AUCpo | Random | 3.15 | 0.09 | 0.67 | 0.02 | 1.02 | 0.08 | 0.47 | 0.03 | 0.64 | 0.02 |
| AutoGluon | Mouse | CLp | Random | 2.09 | 0.04 | 0.43 | 0.01 | 1.00 | 0.05 | 0.48 | 0.02 | 0.65 | 0.01 |
| AutoGluon | Mouse | F | Random | 2.28 | 0.07 | 0.52 | 0.01 | 1.42 | 0.03 | 0.15 | 0.06 | 0.31 | 0.04 |
| AutoGluon | Mouse | Vss | Random | 1.98 | 0.02 | 0.40 | 0.01 | 0.97 | 0.03 | 0.47 | 0.02 | 0.65 | 0.02 |
| AutoGluon | Mouse | AUCiv | Scaffold | 2.25 | 0.06 | 0.47 | 0.03 | 0.97 | 0.06 | 0.43 | 0.04 | 0.60 | 0.04 |
| AutoGluon | Mouse | AUCpo | Scaffold | 3.50 | 0.23 | 0.71 | 0.04 | 1.03 | 0.10 | 0.38 | 0.05 | 0.56 | 0.04 |
| AutoGluon | Mouse | CLp | Scaffold | 2.21 | 0.08 | 0.46 | 0.03 | 0.99 | 0.06 | 0.41 | 0.03 | 0.58 | 0.04 |
| AutoGluon | Mouse | F | Scaffold | 2.33 | 0.22 | 0.54 | 0.07 | 1.50 | 0.19 | 0.14 | 0.02 | 0.26 | 0.05 |
| AutoGluon | Mouse | Vss | Scaffold | 2.09 | 0.07 | 0.42 | 0.02 | 0.98 | 0.08 | 0.41 | 0.05 | 0.58 | 0.04 |
| AutoGluon | Mouse | AUCiv | SCINS | 2.49 | 0.23 | 0.52 | 0.05 | 0.94 | 0.13 | 0.27 | 0.09 | 0.45 | 0.05 |
| AutoGluon | Mouse | AUCpo | SCINS | 3.99 | 0.24 | 0.77 | 0.02 | 0.91 | 0.07 | 0.28 | 0.07 | 0.46 | 0.05 |
| AutoGluon | Mouse | CLp | SCINS | 2.40 | 0.16 | 0.50 | 0.04 | 1.06 | 0.13 | 0.28 | 0.08 | 0.46 | 0.06 |
| AutoGluon | Mouse | F | SCINS | 2.44 | 0.16 | 0.56 | 0.05 | 1.51 | 0.06 | 0.06 | 0.11 | 0.18 | 0.06 |
| AutoGluon | Mouse | Vss | SCINS | 2.20 | 0.11 | 0.45 | 0.03 | 1.00 | 0.04 | 0.31 | 0.06 | 0.50 | 0.04 |
| AutoGluon | Mouse | AUCiv | Time | 2.67 | | 0.58 | | 0.61 | | 0.00 | | 0.26 | |
| AutoGluon | Mouse | AUCpo | Time | 4.69 | | 0.85 | | 0.63 | | 0.06 | | 0.24 | |
| AutoGluon | Mouse | CLp | Time | 2.62 | | 0.56 | | 1.36 | | −0.01 | | 0.23 | |
| AutoGluon | Mouse | F | Time | 2.43 | | 0.53 | | 1.44 | | −0.05 | | 0.09 | |
| AutoGluon | Mouse | Vss | Time | 2.34 | | 0.48 | | 1.24 | | 0.24 | | 0.43 | |
| ChemProp | Mouse | AUCiv | Random | 2.29 | 0.13 | 0.48 | 0.04 | 1.00 | 0.04 | 0.42 | 0.05 | 0.61 | 0.04 |
| ChemProp | Mouse | AUCpo | Random | 3.49 | 0.12 | 0.71 | 0.02 | 1.11 | 0.10 | 0.38 | 0.04 | 0.58 | 0.02 |
| ChemProp | Mouse | CLp | Random | 2.24 | 0.06 | 0.46 | 0.02 | 1.03 | 0.05 | 0.40 | 0.04 | 0.60 | 0.03 |
| ChemProp | Mouse | F | Random | 2.39 | 0.08 | 0.54 | 0.02 | 1.38 | 0.20 | 0.11 | 0.04 | 0.28 | 0.04 |
| ChemProp | Mouse | Vss | Random | 2.08 | 0.07 | 0.42 | 0.03 | 1.02 | 0.08 | 0.40 | 0.06 | 0.60 | 0.04 |
| ChemProp | Mouse | AUCiv | Scaffold | 2.46 | 0.08 | 0.51 | 0.03 | 0.97 | 0.12 | 0.32 | 0.07 | 0.53 | 0.05 |
| ChemProp | Mouse | AUCpo | Scaffold | 3.78 | 0.30 | 0.75 | 0.04 | 0.98 | 0.09 | 0.32 | 0.05 | 0.52 | 0.04 |
| ChemProp | Mouse | CLp | Scaffold | 2.38 | 0.06 | 0.49 | 0.02 | 0.98 | 0.15 | 0.32 | 0.08 | 0.54 | 0.06 |
| ChemProp | Mouse | F | Scaffold | 2.45 | 0.26 | 0.56 | 0.09 | 1.44 | 0.33 | 0.08 | 0.03 | 0.24 | 0.05 |
| ChemProp | Mouse | Vss | Scaffold | 2.13 | 0.08 | 0.43 | 0.03 | 0.98 | 0.04 | 0.37 | 0.05 | 0.57 | 0.04 |
| ChemProp | Mouse | AUCiv | SCINS | 2.56 | 0.10 | 0.53 | 0.03 | 1.01 | 0.14 | 0.23 | 0.16 | 0.47 | 0.11 |
| ChemProp | Mouse | AUCpo | SCINS | 4.09 | 0.23 | 0.79 | 0.03 | 0.95 | 0.08 | 0.25 | 0.07 | 0.49 | 0.05 |
| ChemProp | Mouse | CLp | SCINS | 2.49 | 0.17 | 0.52 | 0.04 | 1.03 | 0.11 | 0.23 | 0.11 | 0.46 | 0.09 |
| ChemProp | Mouse | F | SCINS | 2.43 | 0.04 | 0.55 | 0.03 | 1.34 | 0.14 | 0.07 | 0.05 | 0.20 | 0.05 |
| ChemProp | Mouse | Vss | SCINS | 2.26 | 0.11 | 0.47 | 0.04 | 0.94 | 0.05 | 0.26 | 0.05 | 0.49 | 0.05 |
| ChemProp | Mouse | AUCiv | Time | 2.45 | | 0.52 | | 0.80 | | 0.21 | | 0.38 | |
| ChemProp | Mouse | AUCpo | Time | 4.39 | | 0.81 | | 0.65 | | 0.14 | | 0.36 | |
| ChemProp | Mouse | CLp | Time | 2.48 | | 0.53 | | 1.31 | | 0.09 | | 0.30 | |
| ChemProp | Mouse | F | Time | 2.23 | | 0.51 | | 1.39 | | 0.11 | | 0.33 | |
| ChemProp | Mouse | Vss | Time | 2.32 | | 0.47 | | 1.06 | | 0.26 | | 0.47 | |
| GP | Mouse | AUCiv | Random | 2.11 | 0.07 | 0.44 | 0.02 | 1.01 | 0.06 | 0.50 | 0.03 | 0.67 | 0.02 |
| GP | Mouse | AUCpo | Random | 3.11 | 0.09 | 0.66 | 0.02 | 1.03 | 0.06 | 0.48 | 0.04 | 0.66 | 0.02 |
| GP | Mouse | CLp | Random | 2.08 | 0.02 | 0.43 | 0.01 | 1.00 | 0.04 | 0.48 | 0.03 | 0.66 | 0.01 |
| GP | Mouse | F | Random | 2.23 | 0.08 | 0.50 | 0.02 | 1.39 | 0.10 | 0.20 | 0.03 | 0.36 | 0.01 |
| GP | Mouse | Vss | Random | 1.98 | 0.03 | 0.40 | 0.02 | 1.00 | 0.03 | 0.47 | 0.04 | 0.65 | 0.02 |
| GP | Mouse | AUCiv | Scaffold | 2.29 | 0.06 | 0.48 | 0.03 | 1.00 | 0.06 | 0.41 | 0.03 | 0.58 | 0.03 |
| GP | Mouse | AUCpo | Scaffold | 3.48 | 0.20 | 0.71 | 0.04 | 1.00 | 0.09 | 0.39 | 0.04 | 0.58 | 0.03 |
| GP | Mouse | CLp | Scaffold | 2.21 | 0.07 | 0.46 | 0.03 | 1.01 | 0.06 | 0.41 | 0.02 | 0.58 | 0.02 |
| GP | Mouse | F | Scaffold | 2.32 | 0.20 | 0.53 | 0.07 | 1.40 | 0.17 | 0.15 | 0.01 | 0.30 | 0.03 |
| GP | Mouse | Vss | Scaffold | 2.08 | 0.07 | 0.42 | 0.02 | 1.01 | 0.06 | 0.42 | 0.05 | 0.59 | 0.04 |
| GP | Mouse | AUCiv | SCINS | 2.49 | 0.22 | 0.52 | 0.05 | 0.97 | 0.15 | 0.27 | 0.09 | 0.46 | 0.06 |
| GP | Mouse | AUCpo | SCINS | 4.00 | 0.23 | 0.77 | 0.03 | 0.90 | 0.02 | 0.29 | 0.05 | 0.49 | 0.04 |
| GP | Mouse | CLp | SCINS | 2.42 | 0.17 | 0.50 | 0.04 | 1.03 | 0.12 | 0.27 | 0.07 | 0.47 | 0.05 |
| GP | Mouse | F | SCINS | 2.41 | 0.08 | 0.55 | 0.04 | 1.38 | 0.13 | 0.09 | 0.07 | 0.22 | 0.06 |
| GP | Mouse | Vss | SCINS | 2.19 | 0.09 | 0.44 | 0.03 | 1.00 | 0.03 | 0.33 | 0.07 | 0.51 | 0.06 |
| GP | Mouse | AUCiv | Time | 2.57 | | 0.55 | | 0.72 | | 0.09 | | 0.29 | |
| GP | Mouse | AUCpo | Time | 4.69 | | 0.86 | | 0.52 | | 0.05 | | 0.33 | |
| GP | Mouse | CLp | Time | 2.57 | | 0.55 | | 1.38 | | 0.02 | | 0.24 | |
| GP | Mouse | F | Time | 2.26 | | 0.49 | | 1.13 | | 0.06 | | 0.20 | |
| GP | Mouse | Vss | Time | 2.36 | | 0.48 | | 1.33 | | 0.24 | | 0.44 | |
| AutoGluon | Rat | AUCiv | Random | 1.97 | 0.05 | 0.39 | 0.02 | 0.98 | 0.04 | 0.52 | 0.03 | 0.68 | 0.01 |
| AutoGluon | Rat | AUCpo | Random | 3.45 | 0.15 | 0.70 | 0.03 | 1.05 | 0.06 | 0.43 | 0.02 | 0.61 | 0.02 |
| AutoGluon | Rat | CLp | Random | 2.00 | 0.02 | 0.40 | 0.01 | 0.99 | 0.04 | 0.51 | 0.03 | 0.67 | 0.02 |
| AutoGluon | Rat | F | Random | 2.59 | 0.13 | 0.63 | 0.05 | 1.78 | 0.22 | 0.16 | 0.05 | 0.30 | 0.05 |
| AutoGluon | Rat | Vss | Random | 1.93 | 0.04 | 0.38 | 0.01 | 0.97 | 0.03 | 0.53 | 0.02 | 0.68 | 0.01 |
| AutoGluon | Rat | AUCiv | Scaffold | 2.09 | 0.10 | 0.43 | 0.03 | 0.94 | 0.07 | 0.43 | 0.07 | 0.61 | 0.07 |
| AutoGluon | Rat | AUCpo | Scaffold | 3.73 | 0.14 | 0.74 | 0.02 | 0.99 | 0.13 | 0.37 | 0.06 | 0.55 | 0.06 |
| AutoGluon | Rat | CLp | Scaffold | 2.12 | 0.08 | 0.43 | 0.02 | 1.02 | 0.06 | 0.43 | 0.05 | 0.61 | 0.05 |
| AutoGluon | Rat | F | Scaffold | 2.65 | 0.18 | 0.64 | 0.07 | 1.86 | 0.25 | 0.13 | 0.05 | 0.25 | 0.06 |
| AutoGluon | Rat | Vss | Scaffold | 2.05 | 0.05 | 0.41 | 0.02 | 0.99 | 0.07 | 0.44 | 0.04 | 0.61 | 0.03 |
| AutoGluon | Rat | AUCiv | SCINS | 2.25 | 0.11 | 0.47 | 0.03 | 0.94 | 0.09 | 0.31 | 0.10 | 0.50 | 0.09 |
| AutoGluon | Rat | AUCpo | SCINS | 4.27 | 0.23 | 0.80 | 0.04 | 1.04 | 0.18 | 0.25 | 0.05 | 0.44 | 0.05 |
| AutoGluon | Rat | CLp | SCINS | 2.29 | 0.06 | 0.48 | 0.02 | 1.03 | 0.13 | 0.30 | 0.09 | 0.50 | 0.05 |
| AutoGluon | Rat | F | SCINS | 2.77 | 0.30 | 0.66 | 0.07 | 1.81 | 0.31 | 0.09 | 0.06 | 0.21 | 0.04 |
| AutoGluon | Rat | Vss | SCINS | 2.24 | 0.13 | 0.46 | 0.04 | 1.03 | 0.08 | 0.32 | 0.03 | 0.49 | 0.03 |
| AutoGluon | Rat | AUCiv | Time | 2.89 | | 0.62 | | 0.67 | | 0.03 | | 0.18 | |
| AutoGluon | Rat | AUCpo | Time | 4.06 | | 0.79 | | 0.78 | | 0.06 | | 0.22 | |
| AutoGluon | Rat | CLp | Time | 2.77 | | 0.60 | | 1.43 | | −0.01 | | 0.13 | |
| AutoGluon | Rat | F | Time | 2.48 | | 0.69 | | 1.92 | | −0.16 | | −0.07 | |
| AutoGluon | Rat | Vss | Time | 3.31 | | 0.67 | | 1.15 | | 0.11 | | 0.20 | |
| ChemProp | Rat | AUCiv | Random | 2.07 | 0.04 | 0.41 | 0.01 | 1.02 | 0.10 | 0.47 | 0.02 | 0.65 | 0.02 |
| ChemProp | Rat | AUCpo | Random | 3.93 | 0.21 | 0.77 | 0.03 | 0.97 | 0.09 | 0.33 | 0.03 | 0.52 | 0.03 |
| ChemProp | Rat | CLp | Random | 2.12 | 0.06 | 0.43 | 0.02 | 1.03 | 0.08 | 0.42 | 0.03 | 0.63 | 0.03 |
| ChemProp | Rat | F | Random | 2.71 | 0.16 | 0.64 | 0.05 | 1.57 | 0.26 | 0.13 | 0.11 | 0.33 | 0.03 |
| ChemProp | Rat | Vss | Random | 2.02 | 0.03 | 0.40 | 0.01 | 1.01 | 0.11 | 0.48 | 0.02 | 0.66 | 0.02 |
| ChemProp | Rat | AUCiv | Scaffold | 2.15 | 0.08 | 0.44 | 0.02 | 1.00 | 0.05 | 0.41 | 0.07 | 0.61 | 0.06 |
| ChemProp | Rat | AUCpo | Scaffold | 4.18 | 0.20 | 0.79 | 0.02 | 1.06 | 0.07 | 0.27 | 0.04 | 0.47 | 0.04 |
| ChemProp | Rat | CLp | Scaffold | 2.20 | 0.11 | 0.46 | 0.03 | 1.09 | 0.10 | 0.36 | 0.05 | 0.58 | 0.05 |
| ChemProp | Rat | F | Scaffold | 2.87 | 0.13 | 0.66 | 0.04 | 1.57 | 0.31 | 0.08 | 0.05 | 0.25 | 0.03 |
| ChemProp | Rat | Vss | Scaffold | 2.13 | 0.08 | 0.43 | 0.02 | 1.01 | 0.11 | 0.40 | 0.04 | 0.61 | 0.04 |
| ChemProp | Rat | AUCiv | SCINS | 2.32 | 0.11 | 0.48 | 0.03 | 0.95 | 0.15 | 0.28 | 0.12 | 0.52 | 0.08 |
| ChemProp | Rat | AUCpo | SCINS | 4.74 | 0.58 | 0.86 | 0.07 | 1.02 | 0.14 | 0.15 | 0.13 | 0.39 | 0.11 |
| ChemProp | Rat | CLp | SCINS | 2.35 | 0.07 | 0.49 | 0.02 | 1.01 | 0.08 | 0.26 | 0.12 | 0.51 | 0.06 |
| ChemProp | Rat | F | SCINS | 3.09 | 0.38 | 0.71 | 0.06 | 1.97 | 0.53 | 0.02 | 0.08 | 0.20 | 0.08 |
| ChemProp | Rat | Vss | SCINS | 2.23 | 0.17 | 0.46 | 0.05 | 1.00 | 0.06 | 0.33 | 0.07 | 0.56 | 0.05 |
| ChemProp | Rat | AUCiv | Time | 2.82 | | 0.61 | | 0.68 | | 0.05 | | 0.26 | |
| ChemProp | Rat | AUCpo | Time | 3.99 | | 0.77 | | 0.84 | | 0.10 | | 0.27 | |
| ChemProp | Rat | CLp | Time | 2.68 | | 0.58 | | 1.89 | | 0.03 | | 0.36 | |
| ChemProp | Rat | F | Time | 2.63 | | 0.72 | | 1.15 | | −0.24 | | −0.06 | |
| ChemProp | Rat | Vss | Time | 2.99 | | 0.62 | | 1.18 | | 0.22 | | 0.39 | |
| GP | Rat | AUCiv | Random | 2.00 | 0.07 | 0.40 | 0.02 | 1.02 | 0.05 | 0.51 | 0.04 | 0.68 | 0.02 |
| GP | Rat | AUCpo | Random | 3.47 | 0.19 | 0.70 | 0.04 | 1.03 | 0.07 | 0.43 | 0.03 | 0.61 | 0.03 |
| GP | Rat | CLp | Random | 2.01 | 0.04 | 0.40 | 0.01 | 0.99 | 0.04 | 0.50 | 0.03 | 0.67 | 0.02 |
| GP | Rat | F | Random | 2.55 | 0.10 | 0.61 | 0.05 | 1.64 | 0.13 | 0.19 | 0.05 | 0.35 | 0.01 |
| GP | Rat | Vss | Random | 1.96 | 0.04 | 0.39 | 0.01 | 0.99 | 0.04 | 0.51 | 0.03 | 0.68 | 0.01 |
| GP | Rat | AUCiv | Scaffold | 2.09 | 0.09 | 0.43 | 0.02 | 0.98 | 0.05 | 0.43 | 0.08 | 0.62 | 0.06 |
| GP | Rat | AUCpo | Scaffold | 3.75 | 0.18 | 0.74 | 0.03 | 1.01 | 0.16 | 0.37 | 0.06 | 0.55 | 0.04 |
| GP | Rat | CLF | Scaffold | 2.15 | 0.08 | 0.44 | 0.02 | 1.03 | 0.08 | 0.41 | 0.05 | 0.60 | 0.05 |
| GP | Rat | F | Scaffold | 2.68 | 0.19 | 0.64 | 0.06 | 1.71 | 0.18 | 0.13 | 0.05 | 0.28 | 0.04 |
| GP | Rat | Vss | Scaffold | 2.06 | 0.07 | 0.42 | 0.02 | 1.02 | 0.04 | 0.43 | 0.05 | 0.61 | 0.04 |
| GP | Rat | AUCiv | SCINS | 2.28 | 0.08 | 0.47 | 0.02 | 0.99 | 0.11 | 0.30 | 0.11 | 0.50 | 0.08 |
| GP | Rat | AUCpo | SCINS | 4.21 | 0.32 | 0.79 | 0.05 | 0.98 | 0.17 | 0.27 | 0.05 | 0.46 | 0.05 |
| GP | Rat | CLP | SCINS | 2.32 | 0.07 | 0.48 | 0.02 | 1.03 | 0.12 | 0.28 | 0.07 | 0.49 | 0.04 |
| GP | Rat | F | SCINS | 2.83 | 0.26 | 0.67 | 0.07 | 1.75 | 0.28 | 0.10 | 0.06 | 0.23 | 0.06 |
| GP | Rat | Vss | SCINS | 2.26 | 0.11 | 0.47 | 0.04 | 1.02 | 0.07 | 0.30 | 0.04 | 0.49 | 0.03 |
| GP | Rat | AUCiv | Time | 2.82 | | 0.60 | | 0.74 | | 0.09 | | 0.24 | |
| GP | Rat | AUCpo | Time | 3.75 | | 0.75 | | 0.61 | | 0.14 | | 0.37 | |
| GP | Rat | CLp | Time | 2.71 | | 0.58 | | 1.28 | | 0.04 | | 0.18 | |
| GP | Rat | F | Time | 2.24 | | 0.62 | | 1.31 | | −0.10 | | 0.02 | |
| GP | Rat | Vss | Time | 3.33 | | 0.67 | | 1.09 | | 0.10 | | 0.20 | |

1. A computer-implemented method of generating a machine-learning model configured to predict the value of one or more pharmacokinetic parameters of a pharmaceutical compound having a chemical structure, the computer-implemented method comprising:
receiving training data comprising a plurality of records, each record comprising:
data encoding the chemical structure of a pharmaceutical compound; and
the values of one or more pharmacokinetic parameters obtained from measurements taken from a human or animal subject after administration of the compound to the human or animal subject; and
training the machine-learning model using the training data.

2. A computer-implemented method according to claim 1, wherein:
the one or more pharmacokinetic parameters comprise one or more of: plasma clearance (CLp); volume of distribution at steady state (Vss); oral bioavailability (F); area under the plasma concentration-time curve after intravenous dosing (AUCiv); area under the plasma concentration-time curve after oral dosing (AUCpo).

3. A computer-implemented method according to claim 1, further comprising:
preprocessing the training data before the step of training the machine-learning model.

4. A computer-implemented method according to claim 3, wherein:
preprocessing the training data comprises one or more of the following:
(a) removing records for which dosage levels were greater than a predetermined maximum threshold dosage level;
(b) extrapolating AUCpo or AUCiv values from zero to infinity, and for those records for which the extrapolation factor is greater than a predetermined extrapolation factor threshold, replacing the extrapolated value with the observed zero-to-last measurement;
(c) removing records for which measurements of the value of the one or more pharmacokinetic parameters were obtained from a bodily fluid other than blood or plasma;
(d) removing records for which the molecular weight of the pharmaceutical compound is greater than a predetermined molecular weight threshold;
(e) removing records for which the value of the oral bioavailability parameter is greater than a predetermined maximum oral bioavailability threshold;
(f) preprocessing the training data comprises normalizing the distribution of values of one or more pharmaceutical parameter
(g) standardizing the chemical structures of the pharmaceutical compounds in the training data;
(h) removing pharmaceutical compounds whose chemical structures include fewer than three atoms;
(i) removing pharmaceutical compounds whose chemical structure display significant secondary structure.

5. A computer-implemented method according to claim 1, wherein:
the machine-learning model comprises one or more of a gradient boosting model, an automatic machine-learning model, a Gaussian process regression (GPR) model, a support vector regression (SVR) model, or a random forest model.

6. A computer-implemented method according to claim 5, wherein:
the data encoding the chemical structure of each pharmaceutical compound comprises one or more molecular fingerprints generated based on the chemical structure of that pharmaceutical compound.

7. A computer-implemented method according to claim 6, wherein:
the fingerprint is an extended-connectivity fingerprint (ECFP4 or ECFP6) or a functional-connectivity fingerprint (FCFP4 or FCFP6); a Morgan fingerprint; a Morgan2 fingerprint; or a MAP4 fingerprint.

8. A computer-implemented method according to claim 1, wherein:
the machine-learning model is a neural network.

9. A computer-implemented method according to claim 8, wherein:
the neural network is a graph neural network.

10. A computer-implemented method according to claim 9, wherein:
the graph machine-learning model comprises one or more of: a graph convolutional neural network (GCNN); Attentive Fingerprints; and ChemProp.

11. A computer-implemented method according to claim 9, wherein:
the data encoding the chemical structure of each pharmaceutical compound comprises a graph structure.

12. A computer-implemented method according to claim 8, wherein:
the neural network comprises:
a first sub-network configured to generate a representation of the chemical structure in terms of hidden variables; and
a second sub-network configured to determine the values of the one or more pharmacokinetic parameters based on the generated representation in terms of the hidden variables.

13. A computer-implemented method according to claim 8, further comprising:
receiving pretraining data, the pretraining data comprising a plurality of records, each record comprising:
data encoding the chemical structure of a pharmaceutical compound; and
the values of one or more parameters obtained from in vitro measurements or the values of one or more parameters obtained using machine-learning predictions of in vitro measurements; and
pretraining the machine-learning model using the pretraining data before training the machine-learning model using the training data.

14. A computer-implemented method according to claim 13, wherein:
pretraining the machine-learning model comprises:
training the first sub-network and the second sub-network using the pretraining data; and
reducing the learning rate of the first sub-network; and
training the machine-learning model comprises training the neural network using the training data.

15. A computer-implemented method according to claim 14, wherein:
reducing the learning rate of the first sub-network comprises freezing the first sub-network, and training the machine-learning model comprises training the second sub-network of the neural network using the training data.

16. A computer-implemented method according to claim 13, wherein:
the in vitro measurements comprise one or more of kinetic solubility; permeability across the artificial membrane (PAMPA); hepatocyte in vitro clearance (Clint) and microsomal Clint.

17. A computer-implemented method according to claim 16, wherein:
either:
the pharmacokinetic parameter is CLp and the in vitro measurements comprise one or more of microsomal Clint and hepatocyte Clint; or
the pharmacokinetic parameter is F and
the in vitro measurements comprise one or more of kinetic solubility and PAMPA

18. A computer-implemented method of predicting a pharmacokinetic property of a pharmaceutical compound having a chemical structure, the computer-implemented method comprising:
receiving input data encoding the chemical structure of the compound;
applying a machine-learning model generated using the computer-implemented method of claim 1 to the input data, the machine-learning model configured to generate an output comprising a predicted value of one or more pharmacokinetic parameters of the pharmaceutical compound, wherein the machine-learning model has been trained using training data comprising a plurality of records, each record comprising data encoding the chemical structure of a compound, and the values of one or more pharmacokinetic parameters obtained from measurements taken from a human or animal subject after administration of the compound to the human or animal subject; and
outputting the one or more predicted values of the pharmacokinetic parameters of the pharmaceutical compound.

19. A computer-implemented method according to claim 18, wherein the computer-implemented method is a method of selecting one or more pharmaceutical compounds during a drug design process, and wherein:
the input data comprises data encoding the chemical structure of a pharmaceutical compound of interest during a drug design process;
calculating one or more explainability values indicating a relationship between the one or more predicted pharmacokinetic parameter values and the pharmaceutical compound; and,
displaying the one or more predicted pharmacokinetic parameter values and a representation of the one or more explainability values.

20. A computer-implemented method according to claim 19, wherein calculating the one or more explainability values comprises:
calculating an importance value for each feature of the input data using a SHAP algorithm; and
calculating the one or more explainability values from the importance values.

21. A computer-implemented method according to claim 20, wherein each explainability value indicates a relationship between a respective atom or bond within the chemical structure of the pharmaceutical compound and the one or more predicted pharmacokinetic properties, and wherein calculating the explainability value for a given atom or bond comprises combining the importance values associated with the respective atom or bond.

22. A computer-implemented method according to claim 21, wherein the importance values associated with a respective atom or bond correspond to importance values of features of the input data for which the atom or bond is considered present.

23. A computer-implemented method according to claim 22 wherein the input data comprises a molecular fingerprint in the form of a vector, and wherein each feature of the input data corresponds to a given component of the vector.

24. A computer-implemented method according to claim 23 wherein the importance values associated with the respective atom or bond correspond to the importance vales of the vector components for which the atom or bond is considered present.

25. A computer-implemented method according to claim 24 wherein the atom or bond is considered present in a vector component if the atom or bond corresponds to a central atom or bond represented by the vector component.

26. A computer-implemented according to claim 25 wherein the atom or bond is considered present in a vector component if the atom or bond is within a predetermined radius around the central atom represented by the vector component.

27. A computer-implemented method according to claim 19, wherein displaying a representation of the explainability values comprises highlighting the atoms and/or bonds which have the highest explainability values.

28. A computer-implemented method according to claim 19 wherein the explainability value indicates the accuracy of the predicted one or more pharmacokinetic properties for a pharmaceutical compound.

29. A computer-implemented method according to claim 28, further comprising:
applying a dimensionality reduction algorithm to the data encoding the chemical structure of the pharmaceutical compounds in the training data to obtain reduced-dimensionality training data points, each reduced dimensionality training data point representing a chemical structure of a pharmaceutical compound in the training data; and,
applying the dimensionality reduction algorithm to the input data encoding the chemical structure of the pharmaceutical compound to obtain a reduced-dimensionality input data point representing a chemical structure of the pharmaceutical compound in the input data; and
wherein,
the explainability value is the reduced-dimensionality input data point.

30. A computer-implemented method according to claim 29, wherein displaying the representation of the explainability value comprises displaying a graph which plots the reduced-dimensionality training data points and the reduced-dimensionality input data point.

31. A computer-implemented method according to claim 28 wherein the dimensionality reduction algorithm is a UMAP dimensionality reduction algorithm.

32. A computer-implemented method of selecting one or more pharmaceutical compounds during a drug design process, the method comprising:
receiving input data comprising data encoding the chemical structures of one or more pharmaceutical compounds of interest during a drug design process;
predicting one or more pharmacokinetic properties of each of the pharmaceutical compounds using the computer-implemented method of claim 18;
and selecting one or more of the pharmaceutical compounds for further investigation based on the one or more predicted values of the pharmacokinetic parameters of each compound.
